SEQUENCE LISTING (88 sequences)

SEQ ID NO: 1 - DNA, 1435 bp, Homo Sapiens
gatgcccttctgagccagaggagcgaccccatcttcctggatgtgaagtatggtcctgatcctgttgaaatcaaattggagtctggtgttgccagtggggaggtggttgaggtgatggagggctccagcatgaccttcttagcggaaacaaagtctcacccaccctgtgcctatacttggtttctccttgactccattctgtctcacaccacgagaacattcaccatccatgctgtgtccagagaacatgagggcctgtacaggtgcttggtgtccaacagtgccacccacctgtccagcctgggtactctgaaggtccgagtacttgaaacactgaccatgcctcaagtcgtgccttcaagcctgaaccttgtggagaatgctaggtctgtggacctgacctgccaaaccgtcaatcagagtgtgaatgtccagtggttcctaagtggccagcccctcctgcccagtgagcacctgcagctgtcagctgacaacaggaccctaatcatccatggcctccagcggaatgacaccgggccctatgcctgtgaggtctggaactggggcagccgggcccggagtgagccccttgagctgaccatcaactatggtcctgaccaagtgcacatcaccagggagtcggcatctgagatgatcagcaccatagaggcagagctcaactccagcctgaccctgcagtgttgggccgagtccaagccaggtgctgagtatcgctggactcttgaacactccaccggggagcacctgggtgagcagctgattatcagggctctgacctgggaacacgacgggatctacaactgcacagcctccaactctctcactggcctggcccgctccacttcagtcctggtcaaggtggtaggtccccagtcctcctccctgtcctcaggggccatcgctggtattgtcatcgggatcctggctgtcattgctgtggcctcagaactgggctattttctctacatcagaaatgccagacggccctcaaggaaaacaacagaggaccccagtcatgagacctcacaacccatcccgaaggaggagcaccccacagagcccagttccgaaagcctgagtcctgagtattgcaatatatcccagcttcagggacggatcagagtcgaactgacgaagctgccttcagcaagccgtagaggcaattctttcagcccctggaagccaccacccaaacctctgatgcccccactcagattggtctccactgtgccaaaaaacatggagtcaatctatgaggagcttgtgaatccagagcccaacacttacatccaaatcaacccctccgtctaatggaagcagagatcttttctccaggagtcctagagaaaccatgcttgatcatatatttaatgacatttattaagtgcatattataagccagtcattctctgtc

SEQ ID NO: 2 - protein, 405 aa, Homo Sapiens
MEGSSMTFLAETKSHPPCAYTWFLLDSILSHTTRTFTIHAVSREHEGLYRCLVSNSATHLSSLGTLKVRVLETLTMPQVVPSSLNLVENARSVDLTCQTVNQSVNVQWFLSGQPLLPSEHLQLSADNRTLIIHGLQRNDTGPYACEVWNWGSRARSEPLELTINYGPDQVHITRESASEMISTIEAELNSSLTLQCWAESKPGAEYRWTLEHSTGEHLGEQLIIRALTWEHDGIYNCTASNSLTGLARSTSVLVKVVGPQSSSLSSGAIAGIVIGILAVIAVASELGYFLYIRNARRPSRKTTEDPSHETSQPIPKEEHPTEPSSESLSPEYCNISQLQGRIRVELTKLPSASRRGNSFSPWKPPPKPLMPPLRLVSTVPKNMESIYEELVNPEPNTYIQINPSV

SEQ ID NO: 3 - protein, 443 aa, Homo Sapiens
DALLSQRSDPIFLDVKYGPDPVEIKLESGVASGEVVEVMEGSSMTFLAETKSHPPCAYTWFLLDSILSHTTRTFTIHAVSREHEGLYRCLVSNSATHLSSLGTLKVRVLETLTMPQVVPSSLNLVENARSVDLTCQTVNQSVNVQWFLSGQPLLPSEHLQLSADNRTLIIHGLQRNDTGPYACEVWNWGSRARSEPLELTINYGPDQVHITRESASEMISTIEAELNSSLTLQCWAESKPGAEYRWTLEHSTGEHLGEQLIIRALTWEHDGIYNCTASNSLTGLARSTSVLVKVVGPQSSSLSSGAIAGIVIGILAVIAVASELGYFLYIRNARRPSRKTTEDPSHETSQPIPKEEHPTEPSSESLSPEYCNISQLQGRIRVELTKLPSASRRGNSFSPWKPPPKPLMPPLRLVSTVPKNMESIYEELVNPEPNTYIQINPSV

SEQ ID NO: 4 - DNA, 3102 bp, Homo Sapiens
atgcataattgtcttgccaaaaatgtcaatgccagttgtgtctgctcagctgacgatcacatccatccctccctgggccgtcgagggggcaacgtcaccctgtctgtccaggggatccctcagaatctcatctcctacaattggctccgaggagcaaccaccaatcaggttacccggatcctcaattttaacttcttcagccatggctacaccctaggaccagcccacactggcagggaaacaggcagagctgatggctccctgatcattattgatgtgcgtgcatctgacaatggcatctacaccctgcatctcatctcttctggtgaaaacagttgccatcatgctatgctacttgtctctgagaagctgcaccagcccccggtggaagcccagaacttggcccctctggagcacacagactccttgaatttgacatgcatttctccaaacaatgacaggacattccagtggtttctgaaccttgaggtgattcaggaaggggatggaccagtaatctccagagatggcagggtcctcaccatccccacagtcacacgcaatgactccagcacctaccactgtgaggccaggaaccacctgggatccaggctcagtgaagccctcgtggttggcgtggcttatggcccggatacccccatcgtgaccgcactggacccagattttgtgattggttccaacctcactctggtctgcttagcctactcccacctccttgcccagtacacatggagcttcagtggggtcaccacatgggagggccagaccctcttcatgcccagtctctccagggcacactcaggggtctacacctgcaaggcctccaactccctttccggcttgcacagcagtatggacaccatcatcactgtctcagagacacttcctcagcccaatgtcacagccagtaacttagccccagtggagcatgtggattccatcagtctgcattgccttcctccaaggagcactgtggccatccgccgggatgtcaatggccagaagctcttcattggtggccacagggagctgtccctggactgcagaacactgactctgtcgaacatcaccaggaatgacacgggggtctaccagtgtgagagctggaactcagccaccagcagcatcagcaaccccactctcatcaaagttacatatggcccagaccctcctatggtcaaccctccagacccagaggtcacagctggggcagccctcaccctgtcctgctttgctgactcaaacccccctgcccagtaccactgggagatggacagaaggccaggccctgccacccagcacctggtcatttctgaggtcactctggaccactcagtcaatgggaagatctggatctcagaggttcctggggatgaactgcagccggccttactcaggaccactattcctgctggaggcatcgcagggattgcctcgagtgtcctgatcagcgtggtgctcacagggactgctggctactgtgttggggtcataaggtcccagcccaggaatcctgtggagttcagctcagcaaggaatttaaggagtcatggaaacaagtatggtgtagaggctgcagggggcaggataatgatggtgatgatgaggatgacgatgacgacaacgatgatgatgatgtgttcctcgctttgtaccgtatggagtcctccagctgcagcccagctcaccctcaatgccaacccacttgatgccacccaaagtgaggatgttgttctgcctgtgtttgggacccccaggacaccccagattcatggcagatccagagagctggccaaaccctccattgcagtcagcccaggcactgccatagagcagaaggacatggtgaccttctactgcaccactaaggacgtcaacattaccatccactgggtttccaacaacctctccgttgtgttccatgagcgcatgcagctgtccaaggatggcaagatcctcaccattctcattgtccagcgggaggactcagggacttaccaatgtgaagctcgagatgcccttctgagccagaggagcgaccccatcttcctggatgtgaagtatggtcctgatcctgttgaaatcaaattggagtctggtgttgccagtggggaggtggttgaggtgatggagggctccagcatgaccttcttagcggaaacaaagtctcacccaccctgtgcctatacttggtttctccttgactccattctgtctcacaccacgagaacattcaccatccatgctgtgtccagagaacatgagggcctgtacaggtgcttggtgtccaacagtgccacccacctgtccagcctgggtactctgaaggtccgagtacttgaaacactgaccatgcctcaagtcgtgccttcaagcctgaaccttgtggagaatgctaggtctgtggacctgacctgccaaaccgtcaatcagagtgtgaatgtccagtggttcctaagtggccagcccctcctgcccagtgagcacctgcagctgtcagctgacaacaggaccctaatcatccatggcctccagcggaatgacacggggccctatgcctgtgaggtctggaactggggcagccgggcccggagtgagccccttgagctgaccatcaactatggtcctgaccaagtgcacatcaccagggagtcggcatctgagatgatcagcaccatagaggcagagctcaactccagcctgaccctgcagtgttgggccgagtccaagccaggtgctgagtatcgctggactcttgaacactccaccggggagcacctgggtgagcagctgattatcagggctctgacctgggaacacgacgggatctacaactgcacagcctccaactctctcactggcctggcccgctccacttcagtcctggtcaaggtggtaggggccatcgctggtattgtcatcgggatcctggctgtcattgctgtggcctcagaactgggctattttctctacatcagaaatgccagacgatgcaaccaccagaccttccagaggagacctatgagggcttggaattggattgccaatgaggtggtactggaccagcacatgctggggtga

SEQ ID NO: 5 - protein, 1033 aa, Homo Sapiens
MHNCLAKNVNASCVCSADDHIHPSLGRRGGNVTLSVQGIPQNLISYNWLRGATTNQVTRILNFNFFSHGYTLGPAHTGRETGRADGSLIIIDVRASDNGIYTLHLISSGENSCHHAMLLVSEKLHQPPVEAQNLAPLEHTDSLNLTCISPNNDRTFQWFLNLEVIQEGDGPVISRDGRVLTIPTVTRNDSSTYHCEARNHLGSRLSEALVVGVAYGPDTPIVTALDPDFVIGSNLTLVCLAYSHLLAQYTWSFSGVTTWEGQTLFMPSLSRAHSGVYTCKASNSLSGLHSSMDTIITVSETLPQPNVTASNLAPVEHVDSISLHCLPPRSTVAIRRDVNGQKLFIGGHRELSLDCRTLTLSNITRNDTGVYQCESWNSATSSISNPTLIKVTYGPDPPMVNPPDPEVTAGAALTLSCFADSNPPAQYHWEMDRRPGPATQHLVISEVTLDHSVNGKIWISEVPGDELQPALLRTTIPAGGIAGIASSVLISVVLTGTAGYCVGVIRSQPRNPVEFSSARNLRSHGNKYGVEAAGGRIMMVMMRMTMTTTMMMMCSSLCTVWSPPAAAQLTLNANPLDATQSEDVVLPVFGTPRTPQIHGRSRELAKPSIAVSPGTAIEQKDMVTFYCTTKDVNITIHWVSNNLSVVFHERMQLSKDGKILTILIVQREDSGTYQCEARDALLSQRSDPIFLDVKYGPDPVEIKLESGVASGEVVEVMEGSSMTFLAETKSHPPCAYTWFLLDSILSHTTRTFTIHAVSREHEGLYRCLVSNSATHLSSLGTLKVRVLETLTMPQVVPSSLNLVENARSVDLTCQTVNQSVNVQWFLSGQPLLPSEHLQLSADNRTLIIHGLQRNDTGPYACEVWNWGSRARSEPLELTINYGPDQVHITRESASEMISTIEAELNSSLTLQCWAESKPGAEYRWTLEHSTGEHLGEQLIIRALTWEHDGIYNCTASNSLTGLARSTSVLVKVVGAIAGIVIGILAVIAVASELGYFLYIRNARRCNHQTFQRRPMRAWNWIANEVVLDQHMLG

SEQ ID NO: 6 - DNA, 37 bp, Homo Sapiens
atgcataattgtcttgccaaaaatgtcaatgccagtt

SEQ ID NO: 7 - protein, 12 aa, Homo Sapiens
MHNCLAKNVNAS

SEQ ID NO: 8 - DNA, 327 bp, Homo Sapiens
gtgtctgctcagctgacgatcacatccatccctccctgggccgtcgagggggcaacgtcaccctgtctgtccaggggatccctcagaatctcatctcctacaattggctccgaggagcaaccaccaatcaggttacccggatcctcaattttaacttcttcagccatggctacaccctaggaccagcccacactggcagggaaacaggcagagctgatggctccctgatcattattgatgtgcgtgcatctgacaatggcatctacaccctgcatctcatctcttctggtgaaaacagttgccatcatgctatgctacttgtctctg

SEQ ID NO: 9 - protein, 108 aa, Homo Sapiens
VCSADDHIHPSLGRRGGNVTLSVQGIPQNLISYNWLRGATTNQVTRILNFNFFSHGYTLGPAHTGRETGRADGSLIIIDVRASDNGIYTLHLISSGENSCHHAMLLVS

SEQ ID NO: 10 - DNA, 279 bp, Homo Sapiens
agaagctgcaccagcccccggtggaagcccagaacttggcccctctggagcacacagactccttgaatttgacatgcatttctccaaacaatgacaggacattccagtggtttctgaaccttgaggtgattcaggaaggggatggaccagtaatctccagagatggcagggtcctcaccatccccacagtcacacgcaatgactccagcacctaccactgtgaggccaggaaccacctgggatccaggctcagtgaagccctcgtggttggcgtggctt

SEQ ID NO: 11 - protein, 92 aa, Homo Sapiens
KLHQPPVEAQNLAPLEHTDSLNLTCISPNNDRTFQWFLNLEVIQEGDGPVISRDGRVLTIPTVTRNDSSTYHCEARNHLGSRLSEALVVGVA

SEQ ID NO: 12 - DNA, 254 bp, Homo Sapiens
atggcccggatacccccatcgtgaccgcactggacccagattttgtgattggttccaacctcactctggctgcttagcctactcccacctccttgcccagtacacatggagcttcagtggggtcaccacatgggagggccagaccctcttcatgcccagtctctccagggcacactcaggggtctacacctgcaaggcctccaactccctttccggcttgcacagcagtatggacaccatcatcactgtctcag

SEQ ID NO: 13 - protein, 84 aa, Homo Sapiens
GPDTPIVTALDPDFVIGSNLTLVCLAYSHLLAQYTWSFSGVTTWEGQTLFMPSLSRAHSGVYTCKASNSLSGLHSSMDTIITVS

SEQ ID NO: 14 - DNA, 279 bp, Homo Sapiens
agacacttcctcagcccaatgtcacagccagtaacttagccccagtggagcatgtggattccatcagtctgcattgccttcctccaaggagcactgtggccatccgccgggatgtcaatggccagaagctcttcattggtggccacagggagctgtccctggactgcagaacactgactctgtcgaacatcaccaggaatgacacgggggtctaccagtgtgagagctggaactcagccaccagcagcatcagcaaccccactctcatcaaagttacat

SEQ ID NO: 15 - protein, 92 aa, Homo Sapiens
TLPQPNVTASNLAPVEHVDSISLHCLPPRSTVAIRRDVNGQKLFIGGHRELSLDCRTLTLSNITRNDTGVYQCESWNSATSSISNPTLIKVT

SEQ ID NO: 16 - DNA, 175 bp, Homo Sapiens
atggcccagaccctcctatggtcaaccctccagacccagaggtcacagctggggcagccctcaccctgtcctgctttgctgactcaaacccccctgcccagtaccactgggagatggacagaaggccaggccctgccacccagcacctggtcatttctgaggtcactctggacca

SEQ ID NO: 17 - protein, 57 aa, Homo Sapiens
GPDPPMVNPPDPEVTAGAALTLSCFADSNPPAQYHWEMDRRPGPATQHLVISEVTLD

SEQ ID NO: 18 - DNA, 29 bp, Homo Sapiens
ctcagtcaatgggaagatctggatctcag

SEQ ID NO: 19 - protein, 9 aa, Homo Sapiens
SVNGKIWIS

SEQ ID NO: 20 - DNA, 143 bp, Homo Sapiens
aggttcctggggatgaactgcagccggccttactcaggaccactattcctgctggaggcatcgcagggattgcctcgagtgtcctgatcagcgtggtgctcacagggactgctggctactgtgttggggtcataaggtcccag

SEQ ID NO: 21 - protein, 47 aa, Homo Sapiens
VPGDELQPALLRTTIPAGGIAGIASSVLISVVLTGTAGYCVGVIRSQ

SEQ ID NO: 22 - DNA, 32 bp, Homo Sapiens
cccaggaatcctgtggagttcagctcagcaag

SEQ ID NO: 23 - protein, 10 aa, Homo Sapiens
PRNPVEFSSA

SEQ ID NO: 24 - DNA, 107 bp, Homo Sapiens
gaatttaaggagtcatggaaacaagtatggtgtagaggctgcagggggcaggataatgatggtgatgatgaggatgacgatgacgacaacgatgatgatgatgtgtt

SEQ ID NO: 25 - protein, 35 aa, Homo Sapiens
NLRSHGNKYGVEAAGGRIMMVMMRMTMTTTMMMMC

SEQ ID NO: 26 - DNA, 144 bp, Homo Sapiens
cctcgctttgtaccgtatggagtcctccagctgcagcccagctcaccctcaatgccaacccacttgatgccacccaaagtgaggatgttgttctgcctgtgtttgggacccccaggacaccccagattcatggcagatccagag

SEQ ID NO: 27 - protein, 47 aa, Homo Sapiens
SLCTVWSPPAAAQLTLNANPLDATQSEDVVLPVFGTPRTPQIHGRSR

SEQ ID NO: 28 - DNA, 276 bp, Homo Sapiens
agctggccaaaccctccattgcagtcagcccaggcactgccatagagcagaaggacatggtgaccttctactgcaccactaaggacgtcaacattaccatccactgggtttccaacaacctctccgttgtgttccatgagcgcatgcagctgtccaaggatggcaagatcctcaccattctcattgtccagcgggaggactcagggacttaccaatgtgaagctcgagatgcccttctgagccagaggagcgaccccatcttcctggatgtgaagt

SEQ ID NO: 29 - protein, 91 aa, Homo Sapiens
LAKPSIAVSPGTAIEQKDMVTFYCTTKDVNITIHWVSNNLSVVFHERMQLSKDGKILTILIVQREDSGTYQCEARDALLSQRSDPIFLDVK

SEQ ID NO: 30 - DNA, 279 bp, Homo Sapiens
atggtcctgatcctgttgaaatcaaattggagtctggtgttgccagtggggaggtggttgaggtgatggagggctccagcatgaccttcttagcggaaacaaagtctcacccaccctgtgcctatacttggtttctccttgactccattctgtctcacaccacgagaacattcaccatccatgctgtgtccagagaacatgagggcctgtacaggtgcttggtgtccaacagtgccacccacctgtccagcctgggtactctgaaggtccgagtacttg

SEQ ID NO: 31 - protein, 92 aa, Homo Sapiens
GPDPVEIKLESGVASGEVVEVMEGSSMTFLAETKSHPPCAYTWFLLDSILSHTTRTFTIHAVSREHEGLYRCLVSNSATHLSSLGTLKVRVL

SEQ ID NO: 32 - DNA, 279 bp, Homo Sapiens
aaacactgaccatgcctcaagtcgtgccttcaagcctgaaccttgtggagaatgctaggtctgtggacctgacctgccaaaccgtcaatcagagtgtgaatgtccagtggttcctaagtggccagcccctcctgcccagtgagcacctgcagctgtcagctgacaacaggaccctaatcatccatggcctccagcggaatgacacggggccctatgcctgtgaggtctggaactggggcagccgggcccggagtgagccccttgagctgaccatcaact

SEQ ID NO: 33 - protein, 92 aa, Homo Sapiens
TLTMPQVVPSSLNLVENARSVDLTCQTVNQSVNVQWFLSGQPLLPSEHLQLSADNRTLIIHGLQRNDTGPYACEVWNWGSRARSEPLELTIN

SEQ ID NO: 34 - DNA, 279 bp, Homo Sapiens
atggtcctgaccaagtgcacatcaccagggagtcggcatctgagatgatcagcaccatagaggcagagctcaactccagcctgaccctgcagtgttgggccgagtccaagccaggtgctgagtatcgctggactcttgaacactccaccggggagcacctgggtgagcagctgattatcagggctctgacctgggaacacgacgggatctacaactgcacagcctccaactctctcactggcctggcccgctccacttcagtcctggtcaaggtggtag

SEQ ID NO: 35 - protein, 92 aa, Homo Sapiens
GPDQVHITRESASEMISTIEAELNSSLTLQCWAESKPGAEYRWTLEHSTGEHLGEQLIIRALTWEHDGIYNCTASNSLTGLARSTSVLVKVV

SEQ ID NO: 36 - DNA, 91 bp, Homo Sapiens
gggccatcgctggtattgtcatcgggatcctggctgtcattgctgtggcctcagaactgggctattttctctacatcagaaatgccagacg

SEQ ID NO: 37 - protein, 29 aa, Homo Sapiens
AIAGIVIGILAVIAVASELGYFLYIRNAR

SEQ ID NO: 38 - DNA, 36 bp, Homo Sapiens
atgcaaccaccagaccttccagaggagacctatgag

SEQ ID NO: 39 - protein, 11 aa, Homo Sapiens
CNHQTFQRRPM

SEQ ID NO: 40 - DNA, 52 bp, Homo Sapiens
ggcttggaattggattgccaatgaggtggtactggaccagcacatgctgggg

SEQ ID NO: 41 - protein, 17 aa, Homo Sapiens
AWNWIANEVVLDQHMLG

SEQ ID NO: 42 - DNA, 118 bp, Homo Sapiens
gtccccagtcctcctccctgtcctcaggggccatcgctggtattgtcatcgggatcctggctgtcattgctgtggcctcagaactgggctattttctctacatcagaaatgccagacg

SEQ ID NO: 43 - protein, 38 aa, Homo Sapiens
PQSSSLSSGAIAGIVIGILAVIAVASELGYFLYIRNAR

SEQ ID NO: 44 - DNA, 86 bp, Homo Sapiens
gccctcaaggaaaacaacagaggaccccagtcatgagacctcacaacccatcccgaaggaggagcaccccacagagcccagttccg

SEQ ID NO: 45 - protein, 28 aa, Homo Sapiens
PSRKTTEDPSHETSQPIPKEEHPTEPSS

SEQ ID NO: 46 - DNA, 62 bp, Homo Sapiens
aaagcctgagtcctgagtattgcaatatatcccagcttcagggacggatcagagtcgaactg

SEQ ID NO: 47 - protein, 20 aa, Homo Sapiens
SLSPEYCNISQLQGRIRVEL

SEQ ID NO: 48 - DNA, 126 bp, Homo Sapiens
acgaagctgccttcagcaagccgtagaggcaattctttcagcccctggaagccaccacccaaacctctgatgcccccactcagattggtctccactgtgccaaaaaacatggagtcaatctatgag

SEQ ID NO: 49 - protein, 42 aa, Homo Sapiens
TKLPSASRRGNSFSPWKPPPKPLMPPLRLVSTVPKNMESIYE

SEQ ID NO: 50 - DNA, 157 bp, Homo Sapiens
gagcttgtgaatccagagcccaacacttacatccaaatcaacccctccgtctaatggaagcagagatcttttctccaggagtcctagagaaaccatgcttgatcatatatttaatgacatttattaagtgcatattataagccagtcattctctgtc

SEQ ID NO: 51 - protein, 17 aa, Homo Sapiens
ELVNPEPNTYIQINPSV

SEQ ID NO: 52 - DNA, 279 bp, Homo Sapiens
aaacactgaccatgcctcaagtcgtgccttcaagcctgaaccttgtggagaatgctaggtctgtggacctgacctgccaaaccgtcaatcagagtgtgaatgtccagtggttcctaagtggccagcccctcctgcccagtgagcacctgcagctgtcagctgacaacaggaccctaatcatccatggcctccagcggaatgacaccgggccctatgcctgtgaggtctggaactggggcagccgggcccggagtgagccccttgagctgaccatcaact

SEQ ID NO: 53 - protein, 12 aa, Homo Sapiens
MQPPDLPEETYE

SEQ ID NO: 54 - DNA, 2147 bp, Homo Sapiens
atcattgctattgactatcgtaatcgcagttcccggtctccagggttcctggacttcccgctctttgtgctgggcagggaaggaaagtgagccctgggggtagagggcagaggtggggtcaaagttactttcctgccaccacccctgtctgtcctgggagctgcagttactgcagtgtgtgtgtgagtttgtagcagggtgggagaggagagaagggaggtggaggggctgcacctggagtgaggactgtatctgtgtgtgcaagccggggcgacagacggcctggcacactgaagtcaggtgccgggacccatggggcctgctgactcatggggacaccactggatgggaatcctgctttcagcctcgctttgtaccgtatggagtcctccagctgcagcccagctcaccctcaatgccaacccacttgatgccacccaaagtgaggatgttgttctgcctgtgtttgggacccccaggacaccccagattcatggcagatccagagagctggccaaaccctccattgcagtcagcccaggcactgccatagagcagaaggacatggtgaccttctactgcaccactaaggacgtcaacattaccatccactgggtttccaacaacctctccattgtgttccatgagcgcatgcagctgtccaaggatggcaagatcctcaccattctcattgtccagcgggaggactcagggacttaccaatgtgaagctcgagatgcccttctgagccagaggagcgaccccatcttcctggatgtgaagtatggtcctgatcctgttgaaatcaaattggagtctggtgttgccagtggggaggtggttgaggtgatggagggctccagcatgaccttcttagcggaaacaaagtctcacccaccctgtgcctatacttggtttctccttgactccattctgtctcacaccacgagaacattcaccatccatgctgtgtccagagaacatgagggcctgtacaggtgcttggtgtccaacagtgccacccacctgtccagcctgggtactctgaaggtccgagtacttgaaacactgaccatgcctcaagtcgtgccttcaagcctgaaccttgtggagaatgctaggtctgtggacctgacctgccaaaccgtcaatcagagtgtgaatgtccagtggttcctaagtggccagcccctcctgcccagtgagcacctgcagctgtcagctgacaacaggaccctaatcatccatggcctccagcggaatgacaccgggccctatgcctgtgaggtctggaactggggcagccgggcccggagtgagccccttgagctgaccatcaactatggtcctgaccaagtgcacatcaccagggagtcggcatctgagatgatcagcaccatagaggcagagctcaactccagcctgaccctgcagtgttgggccgagtccaagccaggtgctgagtatcgctggactcttgaacactccaccggggagcacctgggtgagcagctgattatcagggctctgacctgggaacacgacgggatctacaactgcacagcctccaactctctcactggcctggcccgctccacttcagtcctggtcaaggtggtaggtccccagtcctcctccctgtcctcaggggccatcgctggtattgtcatcgggatcctggctgtcattgctgtggcctcagaactgggctattttctctgcatcagaaatgccagacggccctcaaggaaaacaacagaggaccccagtcatgagacctcacaacccatcccgaaggaggagcaccccacagagcccagttccgaaagcctgagtcctgagtattgcaatatatcccagcttcagggacggatcagagtcgaactgatgcaaccaccagaccttccagaggagacctatgagacgaagctgccttcagcaagccgtagaggcaattctttcagcccctggaagccaccacccaaacctctgatgcccccactcagattggtctccactgtgccaaaaaacatggagtcaatctatgaggtgttggggatgcagcagtgatcaacatcgataaaatgcttttccttatggaattgacatcgtagtggtaggtcggacagtagaccaaataaataatt

SEQ ID NO: 55 - protein, 585 aa, Homo Sapiens
MGPADSWGHHWMGILLSASLCTVWSPPAAAQLTLNANPLDATQSEDVVLPVFGTPRTPQIHGRSRELAKPSIAVSPGTAIEQKDMVTFYCTTKDVNITIHWVSNNLSIVFHERMQLSKDGKILTILIVQREDSGTYQCEARDALLSQRSDPIFLDVKYGPDPVEIKLESGVASGEVVEVMEGSSMTFLAETKSHPPCAYTWFLLDSILSHTTRTFTIHAVSREHEGLYRCLVSNSATHLSSLGTLKVRVLETLTMPQVVPSSLNLVENARSVDLTCQTVNQSVNVQWFLSGQPLLPSEHLQLSADNRTLIIHGLQRNDTGPYACEVWNWGSRARSEPLELTINYGPDQVHITRESASEMISTIEAELNSSLTLQCWAESKPGAEYRWTLEHSTGEHLGEQLIIRALTWEHDGIYNCTASNSLTGLARSTSVLVKVVGPQSSSLSSGAIAGIVIGILAVIAVASELGYFLCIRNARRPSRKTTEDPSHETSQPIPKEEHPTEPSSESLSPEYCNISQLQGRIRVELMQPPDLPEETYETKLPSASRRGNSFSPWKPPPKPLMPPLRLVSTVPKNMESIYEVLGMQQ

SEQ ID NO: 56 - DNA, 364 bp, Homo Sapiens
atcattgctattgactatcgtaatcgcagttcccggtctccagggttcctggacttcccgctctttgtgctgggcagggaaggaaagtgagccctgggggtagagggcagaggtggggtcaaagttactttcctgccaccacccctgtctgtcctgggagctgcagttactgcagtgtgtgtgtgagtttgtagcagggtgggagaggagagaagggaggtggaggggctgcacctggagtgaggactgtatctgtgtgtgcaagccggggcgacagacggcctggcacactgaagtcaggtgccgggacccatggggcctgctgactcatggggacaccactggatgggaatcctgctttcag

SEQ ID NO: 57 - protein, 23 aa, Homo Sapiens
SQVPGPMGPADSWGHHWMGILLS

SEQ ID NO: 58 - DNA, 276 bp, Homo Sapiens
agctggccaaaccctccattgcagtcagcccaggcactgccatagagcagaaggacatggtgaccttctactgcaccactaaggacgtcaacattaccatccactgggtttccaacaacctctccattgtgttccatgagcgcatgcagctgtccaaggatggcaagatcctcaccattctcattgtccagcgggaggactcagggacttaccaatgtgaagctcgagatgcccttctgagccagaggagcgaccccatcttcctggatgtgaagt

SEQ ID NO: 59 - protein, 91 aa, Homo Sapiens
LAKPSIAVSPGTAIEQKDMVTFYCTTKDVNITIHWVSNNLSIVFHERMQLSKDGKILTILIVQREDSGTYQCEARDALLSQRSDPIFLDVK

SEQ ID NO: 60 - DNA, 118 bp, Homo Sapiens
gtccccagtcctcctccctgtcctcaggggccatcgctggtattgtcatcgggatcctggctgtcattgctgtggcctcagaactgggctattttctctgcatcagaaatgccagacg

SEQ ID NO: 61 - protein, 38 aa, Homo Sapiens
PQSSSLSSGAIAGIVIGILAVIAVASELGYFLCIRNAR

SEQ ID NO: 62 - DNA, 98 bp, Homo Sapiens
gtgttggggatgcagcagtgatcaacatcgataaaatgcttttccttatggaattgacatcgtagtggtaggtcggacagtagaccaaataaataatt

SEQ ID NO: 63 - protein, 6 aa, Homo Sapiens
VLGMQQ

SEQ ID NO: 64 - DNA, 1931 bp, Homo Sapiens
ttgaggactgtatctgtgtgtgcaagccggggcgacagacggcctggcacactgaagtcaggtgccgggacccatggggcctgctgactcatggggacaccactggatgggaatcctgctttcagcctcgctttgtaccgtatggagtcctccagctgcagcccagctcaccctcaatgccaacccacttgatgccacccaaagtgaggatgttgttctgcctgtgtttgggacccccaggacaccccagattcatggcagatccagagagctggccaaaccctccattgcagtcagcccaggcactgccatagagcagaaggacatggtgaccttctactgcaccactaaggacgtcaacattaccatccactgggtttccaacaacctctccattgtgttccatgagcgcatgcagctgtccaaggatggcaagatcctcaccattctcattgtccagcgggaggactcagggacttaccaatgtgaagctcgagatgcccttctgagccagaggagcgaccccatcttcctggatgtgaagtatggtcctgatcctgttgaaatcaaattggagtctggtgttgccagtggggaggtggttgaggtgatggagggctccagcatgaccttcttagcggaaacaaagtctcacccaccctgtgcctatacttggtttctccttgactccattctgtctcacaccacgagaacattcaccatccatgctgtgtccagagaacatgagggcctgtacaggtgcttggtgtccaacagtgccacccacctgtccagcctgggtactctgaaggtccgagtacttgaaacactgaccatgcctcaagtcgtgccttcaagcctgaaccttgtggagaatgctaggtctgtggacctgacctgccaaaccgtcaatcagagtgtgaatgtccagtggttcctaagtggccagcccctcctgcccagtgagcacctgcagctgtcagctgacaacaggaccctaatcatccatggcctccagcggaatgacaccgggccctatgcctgtgaggtctggaactggggcagccgggcccggagtgagccccttgagctgaccatcaactatggtcctgaccaagtgcacatcaccagggagtcggcatctgagatgatcagcaccatagaggcagagctcaactccagcctgaccctgcagtgttgggccgagtccaagccaggtgctgagtatcgctggactcttgaacactccaccggggagcacctgggtgagcagctgattatcagggctctgacctgggaacacgacgggatctacaactgcacagcctccaactctctcactggcctggcccgctccacttcagtcctggtcaaggtggtaggtccccagtcctcctccctgtcctcaggggccatcgctggtattgtcatcgggatcctggctgtcattgctgtggcctcagaactgggctattttctctacatcagaaatgccagacggccctcaaggaaaacaacagaggaccccagtcatgagacctcacaacccatcccgaaggaggagcaccccacagagcccagttccgaaagcctgagtcctgagtattgcaatatatcccagcttcagggacggatcagagtcgaactgacgaagctgccttcagcaagccgtagaggcaattctttcagcccctggaagccaccacccaaacctctgatgcccccactcagattggtctccactgtgccaaaaaacatggagtcaatctatgaggagcttgtgaatccagagcccaacacttacatccaaatcaacccctccgtctaatggaagcagagatcttttctccaggagtcctagagaaaccatgcttgatcatatatttaatgacatttattaagtgcatattataagccagtcattctctgtc

SEQ ID NO: 65 - protein, 584 aa, Homo Sapiens [sequence omitted]

SEQ ID NO: 66 - DNA, 2172 bp, Homo Sapiens
gagtttgtagcagggtgggagaggagagaagggaggtggaggggctgcacctggagtgaggactgtatctgtgtgtgcaagccggggcgacagacggcctggcacactgaagtcaggtgccgggacccatggggcctgctgactcatggggacaccactggatgggaatcctgctttcagcctcgctttgtaccgtatggagtcctccagctgcagcccagctcaccctcaatgccaacccacttgatgccacccaaagtgaggatgttgttctgcctgtgtttgggacccccaggacaccccagattcatggcagatccagagagctggccaaaccctccattgcagtcagcccaggcactgccatagagcagaaggacatggtgaccttctactgcaccactaaggacgtcaacattaccatccactgggtttccaacaacctctccattgtgttccatgagcgcatgcagctgtccaaggatggcaagatcctcaccattctcattgtccagcgggaggactcagggacttaccaatgtgaagctcgagatgcccttctgagccagaggagcgaccccatcttcctggatgtgaagtatggtcctgatcctgttgaaatcaaattggagtctggtgttgccagtggggaggtggttgaggtgatggagggctccagcatgaccttcttagcggaaacaaagtctcacccaccctgtgcctatacttggtttctccttgactccattctgtctcacaccacgagaacattcaccatccatgctgtgtccagagaacatgagggcctgtacaggtgcttggtgtccaacagtgccacccacctgtccagcctgggtactctgaaggtccgagtacttgaaacactgaccatgcctcaagtcgtgccttcaagcctgaaccttgtggagaatgctaggtctgtggacctgacctgccaaaccgtcaatcagagtgtgaatgtccagtggttcctaagtggccagcccctcctgcccagtgagcacctgcagctgtcagctgacaacaggaccctaatcatccatggcctccagcggaatgacaccgggccctatgcctgtgaggtctggaactggggcagccgggcccggagtgagccccttgagctgaccatcaactatggtcctgaccaagtgcacatcaccagggagtcggcatctgagatgatcagcaccatagaggcagagctcaactccagcctgaccctgcagtgttgggccgagtccaagccaggtgctgagtatcgctggactcttgaacactccaccggggagcacctgggtgagcagctgattatcagggctctgacctgggaacacgacgggatctacaactgcacagcctccaactctctcactggcctggcccgctccacttcagtcctggtcaaggtggtaggtccccagtcctcctccctgtcctcaggggccatcgctggtattgtcatcgggatcctggctgtcattgctgtggcctcagaactgggctattttctctacatcagaaatgccagacggccctcaaggaaaacaacagaggaccccagtcatgagacctcacaacccatcccgaaggaggagcaccccacagagcccagttccgaaagcctgagtcctgagtattgcaatatatcccagcttcagggacggatcagagtcgaactgacgaagctgccttcagcaagccgtagaggcaattctttcagcccctggaagccaccacccaaacctctgatgcccccactcagattggtctccactgtgccaaaaaacatggagtcaatctatgagatggagtcttgctctgttgcccaggatggagtgtagcaacgtgatcttggctcattgcagcctctgcctcccaggttcaagcgattcttctgcctcagactcctgagtagctgggattacaggcgtgcatcaccatgcctgtctaatttttgtattttttaagtagacacagagttttgccatgttggccagactggtcttgaactcttgacctcatgattggcctgccttagcctcccaaagtactaggattacaggtgtgagccactgcgcctggcctattttctgaatatcttaaacaattagcagaaaaataaatgaagtgaaaaaaggaagacgacct

SEQ ID NO: 67 - protein, 578 aa, Homo Sapiens
MGPADSWGHHWMGILLSASLCTVWSPPAAAQLTLNANPLDATQSEDVVLPVFGTPRTPQIHGRSRELAKPSIAVSPGTAIEQKDMVTFYCTTKDVNITIHWVSNNLSIVFHERMQLSKDGKILTILIVQREDSGTYQCEARDALLSQRSDPIFLDVKYGPDPVEIKLESGVASGEVVEVMEGSSMTFLAETKSHPPCAYTWFLLDSILSHTTRTFTIHAVSREHEGLYRCLVSNSATHLSSLGTLKVRVLETLTMPQVVPSSLNLVENARSVDLTCQTVNQSVNVQWFLSGQPLLPSEHLQLSADNRTLIIHGLQRNDTGPYACEVWNWGSRARSEPLELTINYGPDQVHITRESASEMISTIEAELNSSLTLQCWAESKPGAEYRWTLEHSTGEHLGEQLIIRALTWEHDGIYNCTASNSLTGLARSTSVLVKVVGPQSSSLSSGAIAGIVIGILAVIAVASELGYFLYIRNARRPSRKTTEDPSHETSQPIPKEEHPTEPSSESLSPEYCNISQLQGRIRVELTKLPSASRRGNSFSPWKPPPKPLMPPLRLVSTVPKNMESIYEMESCSVAQDGV

SEQ ID NO: 68 - DNA, 343 bp, Homo Sapiens
atggagtcttgctctgttgcccaggatggagtgtagcaacgtgatcttggctcattgcagcctctgcctcccaggttcaagcgattcttctgcctcagactcctgagtagctgggattacaggcgtgcatcaccatgcctgtctaatttttgtattttttaagtagacacagagttttgccatgttggccagactggtcttgaactcttgacctcatgattggcctgccttagcctcccaaagtactaggattacaggtgtgagccactgcgcctggcctattttctgaatatcttaaacaattagcagaaaaataaatgaagtgaaaaaaggaagacgacct

SEQ ID NO: 69 - protein, 11 aa, Homo Sapiens
MESCSVAQDGV

SEQ ID NO: 70 - DNA, 833 bp, Homo Sapiens
  misc_feature 528,654,720,723,733,734,737,738,744,748,753,765,768,781,786,787,789,791,793,796,797,799,806: n = A,T,C or G
  misc_feature 813,817,818,824,826,828: n = A,T,C or G
tttgtatcccacttactatagggcgaattgggccctctagatgcatgctcgagcggcccgccagtgtgatggatatctgcagaattcgcccttcatcgctggtattgtcatcgggatcctggctgtcattgctgtggcctcagaactgggctattttctctgcatcagaaatgccagacggccctcaaggaaaacaacagaggaccccagtcatgagacctcacaacccatcccgaaggaggagcaccccacagagcccagttccgaaagcctgagtcctgagtattgcaatatatcccagcttcagggacggatcagagtcgaactgatgcaaccaccagaccttccagaggagacctatgagggcttggaattggattgccaatgaggtggtactggacaagggcgaattccagcacactggcggccgttactagtggatccgagctcggtaccaagcttgatgcatagcttgagtattctatagtgtcacctaaatagcttggcggtaatcatggtcatagctgnttcctgtgtgaaattgttatcccgctcacaattccacacaacatacgagccggaagcataaagtgtaaagcctggggtgcctaatgagtgagctaactcacattaattggcgttgcgctcacttgnccgctttccagtcgggaaaacctgtcgtgccagctgcattaatggaatcgggcccaacgcgcgggnganaagccggttnncgnnttgggncgcntcttncgctttcttggntcnctggactcccttncgctnngncntncgnntncggccanccggttntcanntcactncnanaggcc

SEQ ID NO: 71 - protein, 97 aa, Homo Sapiens
IAGIVIGILAVIAVASELGYFLCIRNARRPSRKTTEDPSHETSQPIPKEEHPTEPSSESLSPEYCNISQLQGRIRVELMQPPDLPEETYEGLELDCQ

SEQ ID NO: 72 - DNA, 501 bp, Homo Sapiens
catcgctggtattgtcatcgggatcctggctgtcattgctgtggcctcagaactgggctattttctctgcatcagaaatgccagacggccctcaaggaaaacaacagaggaccccagtcatgagacctcacaacccatcccgaaggaggagcaccccacagagcccagttccgaaagcctgagtcctgagtattgcaatatatcccagcttcagggacggatcagagtcgaactgatgcaaccaccagaccttccagaggagacctatgagacgaagctgccttcagcaagccgtagaggcaattctttcagcccctggaagccaccacccaaacctctgatgcccccactcagattggtctccactgtgccaaaaaacatggagtcaatctatgaggagcttgtgaatccagagcccaacacttacatccaaatcaacccctccgtctaatggaagcagagatcttttctccaggagtcctagagaaaccatgcttgatc

SEQ ID NO: 73 - protein, 149 aa, Homo Sapiens
IAGIVIGILAVIAVASELGYFLCIRNARRPSRKTTEDPSHETSQPIPKEEHPTEPSSESLSPEYCNISQLQGRIRVELMQPPDLPEETYETKLPSASRRGNSFSPWKPPPKPLMPPLRLVSTVPKNMESIYEELVNPEPNTYIQINPSV

SEQ ID NO: 74 - protein, 14 aa, Artificial Sequence
  calmodulin binding site
FLYIRNARRPSRKT

SEQ ID NO: 75 - protein, 6 aa, Artificial Sequence
  minimal calmodulin binding domain
LQGRIR

SEQ ID NO: 76 - protein, 4 aa, Artificial Sequence
  phosphorylation site
SPWK

SEQ ID NO: 77 - protein, 4 aa, Artificial Sequence
  SH2 domain
YCNI

SEQ ID NO: 78 - protein, 4 aa, Artificial Sequence
  SH2 domain
YEEL

SEQ ID NO: 79 - protein, 4 aa, Artificial Sequence
  SH2 domain
YIQI

SEQ ID NO: 80 - protein, 14 aa, Artificial Sequence
  calmodulin binding site
FLCIRNARRPSRKT

SEQ ID NO: 81 - protein, 4 aa, Artificial Sequence
  SH2 domain
YEVL

SEQ ID NO: 82 - DNA, 21 bp, Artificial Sequence
  CEA primer
catcgctggtattgtcatcgg

SEQ ID NO: 83 - DNA, 20 bp, Artificial Sequence
  CEA primer
ggacttcgagcaagagatgg

SEQ ID NO: 84 - DNA, 23 bp, Artificial Sequence
  actin primer
cgtctggcatttctgatgtagag

SEQ ID NO: 85 - DNA, 21 bp, Artificial Sequence
  actin primer
tgaaggtagtttcgtggatgc

SEQ ID NO: 86 - DNA, 24 bp, Artificial Sequence
  primer
ctgccatagagcagaaggacatgg

SEQ ID NO: 87 - DNA, 25 bp, Artificial Sequence
  primer
ggatgattagggtcctgttgtcagg

SEQ ID NO: 88 - protein, 4 aa, Artificial Sequence
  SH2 domain
YEGL

(19) United States
(12) Patent Application Publication
(10) Pub. No.: US 2004/0249145 A1
(43) Pub. Date: Dec. 9, 2004

(54) Cell adhesion-mediating proteins and polynucleotides encoding them

(76) Inventors: Karen A. Stark, Arlington, MA (US); Alix Weaver, Northbridge, MA (US); Heidi M. Hoffmann, Lunenberg, MA (US); Raul Krauss, Newton, MA (US); Kulvinder Singh Saini, Haryana (IN); Dario B. Valenzuela, Houston, TX (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/704,363

(22) Filed: Nov. 7, 2003

Related U.S. Application Data
(63) Continuation of application No. PCT/US02/14457, filed on May 7, 2002.
(60) Provisional application No. 60/289,179, filed on May 7, 2001.
(60) Provisional application No. 60/315,736, filed on Aug. 29, 2001.

(51) Int. Cl. C07K 14/705; C07H 21/04

(52) U.S. Cl. 536/23.5; 435/69.1; 435/320.1; 435/325; 530/350

(57) ABSTRACT

The present invention provides multiple polynucleotide sequences from the same novel gene, the exons comprising the polynucleotide sequences, and the proteins encoded by the polynucleotide sequences. Three splicing variant polynucleotides were isolated from prostate tissue. The polypeptides, including the splicing variants, have a region of hydrophobicity indicative of a transmembrane domain and all three extracellular and cytoplasmic domains.

### RELATED APPLICATIONS

This application continuation of International Application No. PCT/US02/14457, which designated the United States and was filed May 7, 2002, published in English, which claims the benefit of U.S. Provisional Application No. 60/289,179, filed May 7, 2001, and U.S. Provisional Application No. 60/315,736, filed Aug. 29, 2001.

The entire teachings of the above application(s) are incorporated herein by reference.

### BACKGROUND OF THE INVENTION

Tumor markers are an invaluable aid in the diagnosis, treatment and monitoring of cancer. One of the earliest markers discovered, and still a marker of great utility is carcinoembryonic antigen (CEA), a member of the human CEA family of molecules. Antibodies to CEA have proven valuable for the detection of both primary and metastatic colorectal cancer, for monitoring progression of disease and response to treatment, for radiolocalization of tumors and for antibody-mediated therapies (reviews, Hammarstrom, Seminars in Cancer Biology, 9:67-81 (1999); Bidart et al., Clinical Chemistry, 45:1695-1707 (1999)).

Prostate cancer has emerged as the second leading cause of cancer mortality among American men, surpassed only by lung cancer. Advances in the molecular genetics of prostate cancer have led to the hope that new diagnostic and prognostic markers will lead to better “targeted” therapies for individual patients. Tumors can shed CEA into the bloodstream. High serum levels of CEA can be prognostic and are used to detect recurrence of colon cancer post-operatively. Very high serum levels can be indicative of liver metastasis of colon cancer (review, Hammarstrom, Seminars in Cancer Biology, 9:67-81 (1999)). Normal colon also produces CEA where it is primarily secreted into the lumen and thus ends up in the feces. Labeled antibodies to CEA have been used to locate the sites of original colorectal, stomach and breast tumors and metastasises as a prognostic and diagnostic tool (e.g., Goldenberg et al., Cancer, 89:104-15 (2000); Nakopoulou et al., Dis

Colon Rectum, 26:269-74 (1983)), for radioimmunoguided surgery (e.g., Beroux et al., Hepatogastroenterology, 46:3099-108 (1999)), and as an aid in assessment of treatment (e.g., Lechner et al., J Am Coll Surg, 191:511-8 (2000); Yamao et al., Jpn J Clin Oncol, 29:550-5 (1999)). Antibodies to CEA to which a cytotoxic agent, such as high-level radioisotope or nitrous oxide, has been attached can be administered and used as treatment that will specifically target to CEA expressing-tumors (Khare et al., Cancer Research, 61:370-5 (2001); Buchegger et al., Int J Cancer, 41:127-134 (1988)). Nevertheless, minimally-invasive and more sensitive molecular markers of prostate and other cancers are needed which could detect development of the disease and also help in monitoring the therapy for individual patients.

CEA family member CEACAM1 has had limited utility as a marker for prostate cancer (Feuer et al., J Investig Med, 46:66-72 (1988)). The cross reactivity of the antibodies among CEA various members, the frequency of alternative splicing, and changes in expression levels that vary with tumor staging, all had an early confounding effect on the elucidation of the roles of CEA family members in cancer. Therefore, if CEA is to be used in prognosis and treatment of prostate cancer, CEA genes or transcripts thereof showing more specificity or reliable expression in normal or tumor derived prostate tissue is needed.

### SUMMARY OF THE INVENTION

Applicants describe herein prostate specific human CEA transcripts and their use as markers of prostate tissue, both normal and tumor derived. As described above, CEA genes are useful as diagnostic and prognostic markers of colon cancer as well as stomach and breast cancers. As described herein, prostate specific CEA transcripts are provided that can be used in diagnosis, prognosis and treatment of prostate cancer. The successes and limitations of currently available cancer markers underscore both the benefits derived from even limited markers, and the need for novel ones. The advantages offered by early diagnosis, the ability to monitor both progression of the disease and the efficacy of therapy, and targeting of specific treatments to tumor cells clearly demonstrate the usefulness and desirability of additional cancer markers, which could bring about improved patient outcome. Knowledge of the polypeptides that can act as markers, and the oligonucleotides encoding them is needed in order to diagnose and treat cancer in its various stages.

The invention provides novel CEA nucleic acid transcripts and polypeptides encoded by such nucleic acids. The novel nucleic acids share the motif pattern of members of the CEA family. The novel nucleic acids and proteins are useful as biomarkers for identifying cancer cells, cancer prognosis, monitoring progression of cancer and in developing treatments for cancer, in particular prostate cancer.

The invention provides isolated nucleic acid encoding full length human CEA. The nucleic acids comprise SEQ ID NOs: 1, 4, 54, 64, 66, 70, 72 and complementary sequences thereof. Some of the polynucleotides of the present invention are splice variants of the same CEA gene. The invention also includes isolated nucleic acid encoding full length human CEA protein. The polypeptides include SEQ ID NOs: 2, 3, 5, 55, 65, 67, 71 and 73. Nucleic acid sequences encoding the exons of the human CEA DNA are also provided herein. The exons include nucleic acid comprising SEQ ID NOs: 6,8, 10, 12, 14, 16, 18, 20, 22, 24, 26, 28, 30, 32, 34, 36, 38, 40, 42, 44, 46, 48, 50, 52, 56, 60, 62 and 68.

The nucleic acid sequences provided herein (e.g., the exon sequence) can be labeled and used as reporter probes to identify cells expressing the exon sequences. In particular, such reporter probes can be used for histological typing of tissue sections, such as for example, when identifying cells from the prostate. Such probes, singly or in combination, can be used to identify specific splicing variants expressed in cells and tissues. Such exon sequences can also be used for gene therapy to replace mutated sites. Such nucleic acid sequences can be used to express the encoded amino acid sequences. In a further aspect, the invention features an antisense construct comprising all or a portion of any one of the nucleic acid sequences provided herein or combination thereof, where the construct encodes a mRNA that is complementary to a native mRNA, and can bind to and block the translation of that native mRNA. In still further aspect, the invention features a double stranded RNA construct corresponding to all or a portion of any one of the nucleic acid sequences provided herein or combination thereof, where the construct is capable of blocking translation of that native mRNA. The invention also includes isolated nucleic acid that hybridizes to the sequences provided herein under conditions of high stringency. The nucleic acids of the present invention can be operably linked to one or more control sequences to provide an expression vector or construct, which can in turn be transformed into a host cell.

The invention is drawn to isolated polynucleotides selected from the group consisting of: SEQ ID NOs: 1, 54, 64, 66, 70, 72 and polynucleotides complementary to any one of SEQ ID NOs: 1, 54, 64, 66, 70, and 72. The group also includes a polynucleotide encoding a polypeptide sequence selected from the group consisting of: SEQ ID NOs: 2, 3, 5, 55, 65, 67, 71, 73; and polynucleotides that are 90% identical to any one of the polynucleotides of the above-mentioned nucleic acid SEQ ID NOs., using DNA alignment program BLASTN on default parameters, wherein the polynucleotide having 90% identity encodes a CEA protein.

The invention is also drawn to exons of CEA proteins, including an isolated polynucleotide from the group consisting of: SEQ ID NOs: 6, 8, 10, 12, 14, 16, 18, 20, 22, 24,26, 28, 30, 32, 34, 36, 38, 40, 42, 44, 46, 48, 50, and 52. The group also includes a polynucleotide encoding a polypeptide sequence selected from the group consisting of: SEQ ID NOs: 7, 9, 11, 13, 15, 17, 19, 21, 23, 25, 27, 29, 31, 33, 35, 37, 39, 41, 43, 45, 47, 49, 51, 53; and a polynucleotides complementary to any one of the above-mentioned polynucleotide sequences.

The invention further includes methods for producing a CEA polypeptide. The method comprises culturing a host cell transformed with the isolated polynucleotide of the present invention in a suitable culture medium; and isolating the expressed protein from the culture medium. The invention includes proteins produced by the method of the present invention.

The method further includes kits for use in detecting CEA expression in a biological sample. The method comprises at least one oligonucleotide probe which selectively binds under high stringency conditions to an isolated nucleic acid comprising a sequence selected from the group consisting of: SEQ ID NOs: 1, 54, 64, 66, 70, and 72, wherein said probe is detectably labeled.

The invention also includes a method for detecting CEA expression in a biological sample, wherein the biological sample comprises RNA. The method comprises contacting a biological sample with a nucleic acid probe, under conditions such that the nucleic acid probe hybridizes to complementary RNA sequence, if present, in the biological sample. The probe is designed to specifically hybridize any one of SEQ ID NOs: 1, 54, 64, 66, 70, and 72. The specifically hybridized probe is then detected, thereby detecting CEA expression in the biological sample.

The invention also includes CEA polypeptide. The CEA polypeptide is selected from the group consisting of: SEQ ID NOs: 2, 3, 5, 55, 65, 67, 71, 73; polypeptides having 80% identity with any one of SEQ ID NOs: 2, 3, 5, 55, 65, 67, 71, 73 using protein alignment program BLASTP under default conditions; and SEQ ID NOs: 7, 9, 11, 13, 15, 17, 19, 21, 23, 25, 27, 29, 31, 33, 35, 37, 39, 41, 43, 45, 47, 49, 51, and 53.

The invention further includes a purified antibody that selectively binds to a polypeptide of the present invention, or fragments thereof, as well as a purified antigen derived from the polypeptides of the present invention and glycosylated versions thereof.

The present invention is also drawn to a method for detecting CEA polypeptide in a biological sample. The biological sample comprises polypeptides, and the method comprises contacting a biological sample with a CEA specific antibody, under conditions such that the antibody binds to the CEA protein, if present, in the biological sample. The antibody is specific for any one of SEQ ID NOs: 2, 3, 5, 55, 65, 67, 71, and 73. The specifically bound antibody, is then detected, thereby detecting CEA protein in the biological sample.

The invention also provides a method for treatment or prevention of cancer, the method comprising administering antibodies specific for a polypeptide selected from the group consisting of SEQ ID NOs: 2, 3, 5, 55, 65, 67, 71 or 73, fragments thereof or combinations thereof.

In a further embodiment of a method for treatment of cancer, a therapeutic agent comprising a binding partner that can bind to at least one of the polypeptides of SEQ ID NOs: 2, 3, 5, 55 65, 67, 71 and 73 and a therapeutic agent, such as for example, a cytotoxic agent or a radioisotope, conjugated thereto, are provided for administration to a patient in need thereof.

The invention further provides a method for diagnosis of or prognosis of cancer, the method comprising providing a biological sample, such as for example, a tissue biopsy or a plasma sample, and a reporter probe comprising a binding partner that can selectively bind to at least one of the polypeptides of SEQ ID NOs: 2, 3, 5, 55, 65, 67, 71 and 73 conjugated to a reporter molecule such as for example, a fluorescent dye, a radioisotope or an enzyme.

The invention further comprises a method for localizing cells or tissue in a patient comprising administering a reporter probe that is specific for at least one of SEQ ID NOs: 2, 3, 5, 55, 65, 67, 71 and 73 such as for example, described above, to the patient under conditions permitting formation of a complex between the reporter probe and the molecule of SEQ ID NOs: 2, 3, 5, 55, 65, 67, 71 and 73, respectively, and monitoring the location of that reporter probe. Localization of cells is useful, for example, for diagnosis, for determining severity of a cancer, for monitoring efficacy of a treatment, and for surgical preparation.

This invention also provides a method for identifying the binding partners of at least one of the polypeptides of SEQ ID NOs: 2, 3, 5, 55, 65, 67, 71 and 73 and for identifying small molecules that disrupt the interaction of the polypeptide and its binding partners. Such method utilizes protein-protein interaction assays.

Such amino acid sequences can each be used to produce antibodies that when conjugated to a label can be used to detect cells producing proteins that include such polypeptides. Such antibodies used singly or in combination can be used to detect cells and tissues producing specific protein variants or to quantitate the amount of each splice variant by ELISA.

### BRIEF DESCRIPTION OF THE DRAWINGS

FIG. 1 is a Northern analysis of RNA from the indicated tissues using SEQ ID NO: 1 as a probe.

FIG. 2A is a diagram of the exon structure of chromosome 19 and SEQ ID NOs: 1, 54, 64, and 66. Corresponding SEQ ID NOs are given above the boxes representing each exon. An asterisk indicates a single nucleotide polymorphism relative to the chromosomal sequence. A dotted line indicates a partial exon.

FIG. 2B is a diagram of the gene structure of chromosome 19 and SEQ ID NOs: 70 and 72.

FIG. 3 shows the protein structures found in a CEA family member, CEACAM1, compared to SEQ ID NOs: 2, 55, 65 and 67. The extracellular domains of the molecules are identified by letters. “N” indicates an N-terminal V-type immunoglobulin domain, “A” and “B” indicate particular subtypes of C-type immunoglobulin domains. The cell membrane is represented, with the corresponding transmembrane domains and the cytoplasmic domains below the cell membrane. Glycosylation sites on the extracellular domains of the proteins are shown.

FIG. 4 shows a Northern analysis using the full insert of pCEA1 as probe. N: normal prostate RNA; T: prostate tumor RNA; P: pooled RNA, 1-10 are RNAs from ten (10) different individuals. For individuals 5-9, both tumor RNA and RNA from the normal portion of the prostate are present. Sizes of markers are at left given in kilobases. Below the Northern blots are images of the ethidium bromide stained gels demonstrating amount of RNAs loaded into each lane.

FIG. 5A shows the determination of linear range of PCR amplification for PCEA and for beta actin control.

FIG. 5B shows quantities of product obtained for CEA normalized to beta actin controls. Vertical axis is the ratio of CEA concentration to beta actin concentration obtained. Normal prostate tissue samples are grouped at left; prostate tumor samples are grouped at right. Numbers indicate individual patients.

FIG. 6 shows expression of PCEA polypeptides: Lanes 1-3 are is SEQ ID NOs: 65, 55 and 67, respectively. Lane 4 is no template control, sizes in kDa are shown at left for the three black bars indicating the location of molecular weight standards, and dotted arrows indicate the presence of the full-length expressed proteins.

FIG. 7 shows Table 1, listing exons of SEQ ID NOs: 1 and 4.

FIG. 8 shows Table 2, listing exons of SEQ ID NOs: 54 and 64.

FIG. 9 shows Table 3, listing exons of SEQ ID NO: 70.

### DETAILED DESCRIPTION OF THE INVENTION

The present invention is directed to nucleic acid and protein sequences of the human CEA gene family. The human CEA family of molecules are members of the immunoglobulin superfamily and include transmembrane, secreted, and glycosylphosphotidylinositol-membrane-linked molecules. The genes are located on human chromosome 19 in region 19q13 (review, Hammarstrom, 1999, ibid). These molecules function in cell adhesion and cell signaling (review, Obrink, Current Opinion in Cell Biology, 9:616-26 (1997)) suggestive of their role in tumors and particularly in metastasis of colorectal tumors to liver (Gangopadhyay et al., Clin Exp Metastasis, 16:703-12 (1988)). Family members participate in homophilic as well as heterophilic binding with molecules on adjacent cells and can dimerize (Hunter et al., Biochem J, 320:847-53 (1996)). Recently, one member CEACAM1 (biliary glycoprotein, BGP), has been shown to respond to VEGF and trigger angiogenesis, a process that is also crucial for tumor growth (review, Wagener and Ergun, Exptl Cell Res, 261:19-24 (2000)). CEACAM1 also has alternatively spliced cytoplasmic domains that bind calmodulin (Edlund et al., J Biol Chem, 271:1393), participate differentially in signaling (Sadekova et al., Mol Biol Cell, 11:65-77 (2000)) and whose ratios of expression differ in normal and tumor tissue (Turbide et al., Cancer Res, 57:2781-8 (1997)) all of which seem to be important for its function as an inhibitor of tumor growth.

Multiple CEA family members have been shown to be differentially expressed in tumor tissue including up-regulation in gastric carcinoma and squamous cell lung carcinoma, down-regulation in hepatocellular carcinoma and up- or down-regulation in colorectal carcinoma, and to be expressed as well in colon, breast, lung and ovarian carcinoma (reviews, Shively and Beatty, CRC Crit Rev Oncol Hematol, 2:355-399; Hammarstrom, 1999, ibid).

The present invention provides isolated nucleic acids including nucleotide sequences comprising and/or derived from at least of SEQ ID NOs: 1, 4, 54, 64, 66, 70 and 72 and isolated polypeptides encoded thereby comprising or derived from the polypeptides of SEQ ID NOs: 2, 3, 5, 55, 65, 67, 71 and 73. The nucleic acid sequences of the invention include the specifically disclosed sequences of SEQ ID NOs: 1, 4, 54, 64, 66, 70 and 72 splice variants, allelic variants and species homologs of these sequences.

Subsets of the nucleic acid sequences and combinations of the sequences with heterologous sequences are also provided. The sequences comprise consecutive nucleotides from the sequences provided herein but preferably include at least 8-10, and more preferably 9-25, consecutive nucleotides from an novel sequence. Other preferred subsets of the sequences include those encoding one or more of the functional domains or antigenic determinants of the novel proteins and, in particular, may include either normal or mutant sequences. The subsequences provide herein are produced using routine techniques known in the art, for example, by PCR. Primers designed to hybridize the 5′ and 3′ termini of the subsequence of interest can be used to amplify said region using the appropriate sequence provided herein as a template in a standard PCR amplification. The primers can include restriction enzyme recognition sequences to facilitate inserting th fragment into the desired vector. Using no more than routine optimization, one of ordinary skill in the art can amplify any desired nucleic subsequence of the sequences provided herein. Alternatively, desired subsequences can be synthesized using routine in vitro synthesis techniques. Subsequences include the exon sequences, provided by SEQ ID Nos: 6, 8, 10, 12, 14, 16, 18, 20, 22, 24, 26, 28, 30, 32, 34, 36, 38, 40, 42, 44, 46, 48, 50, 52, 56, 60, 62 and 68. The nucleic acid subsequences can be inserted into a suitable vector for propagation amplification, and expression of the encoded protein.

The invention also provides nucleic acid constructs comprising the sequences provided herein or fragments thereof, linked to suitable promoters and selective markers to form cloning vectors, expression vectors, fusion vectors, transgenic constructs, and the like. For example, the isolated polynucleotides and variant polynucleotides encoding the protein and protein variants of the present invention may be operably linked to an expression control sequence such as the pMT2 or pED expression vectors disclosed in Kaufman et al., Nucleic Acid Res, 19:4485-4490 (1991). Many suitable expression control sequences are known in the art. Thus, in accordance with another aspect of the invention, a recombinant vector for transforming a mammalian or invertebrate tissue cell to express a normal or mutant sequence of the present invention, such as for Example 1 of SEQ ID NOs: 2, 3, 5, 55, 65, 67, 71 and 73 in the cells is provided.

The present invention includes compositions comprising one or more of the isolated polynucleotide described herein, as well as vectors and host cells containing such a polynucleotide, and processes for producing the proteins encoded by such a polynucleotide, and their fragments, mutants, species homologs, and allelic variants, through the use of such vectors and host cells. Examples of vectors for insertion of a nucleic acid of the present invention include nucleic acid molecules derived from, for example, a plasmid; a bacteriophage; a mammalian, plant or insect virus; or non-viral vectors such as ligand-nucleic acid conjugates, liposomes or lipid-nucleic acid complexes. It may be desirable that the transferred nucleic acid molecule is operably linked to an expression control sequence to form an expression vector capable of expressing the transferred nucleic acid. The exogenous polynucleotide may be maintained as a non-integrated vector, for example, as a plasmid or alternatively, may be integrated into the host genome.

Isolated polynucleotide of the present invention can encode additional amino acids, as a linker. Such linkers are known to those of skill in the art, for example, the linker can comprise at least one additional codon encoding at least one additional amino acid. Typically the linker comprises one to about twenty or thirty amino acids. The polynucleotide is translated, as is the polynucleotide encoding the protein, resulting in the expression of a protein with at least one additional amino acid residue at the amino or carboxyl terminus of the protein. Importantly, the additional amino acid or amino acids, does not compromise the activity of the protein.

In another embodiment, the present invention provides for host cells that have been transfected or otherwise transformed with one of the nucleic acids of the present invention. Host cells can be prokaryotic or eukaryotic, mammalian, plant or insect, and can exist as single cells or as a collection of cells, such as a cell culture or in a tissue culture or in an organism. Host cells can be derived from normal or diseased tissue from a multicellular organism such as for example, a mammal. Host cell, as used herein, is intended to include not only the original cell that was transformed with a nucleic acid, but also descendants of such a cell, which still contain the nucleic acid sequence.

The present invention is also drawn to CEA proteins and fragments thereof. The CEA protein sequences include SEQ ID NOs: 2, 3, 5, 55, 65, 67, 71 and 73. Fragments of the proteins of the present invention that are capable of exhibiting biological activity and the nucleotide sequences that encode them are also encompassed by the present invention. Such fragments include, but are not limited to, fragments encoded by one or more exons. Such exons are provided in SEQ ID NOs: 6, 8, 10, 12, 14, 16, 18, 20, 22, 24, 26, 28, 30, 32, 34, 36, 38, 40, 42, 44, 46, 48 and 50 and the amino acid sequences encoded thereby include SEQ ID NOs: 7, 9, 11, 13, 15, 17, 19, 21, 23, 25, 27, 29, 31, 33, 35, 37, 39, 41, 43, 45, 47, 49 and 51, respectively. Such exons are also provided by SEQ ID NOs: 52, 56, 58, 60, 62 and 68 and the amino acid sequences encoded thereby include SEQ ID NOs: 33, 57, 59, 61, 63 and 69, respectively. SEQ ID NO: 38 also encodes an alternative peptide that is shown as SEQ ID NO: 53. Fragments of the protein may be in linear form or they may be cyclized using known methods, for example, as described in U.S. Pat. No.: 6,017,878, in H. U. Saragovi et al., Bio/Technology, 10:773-778 (1992); and in R. S. McDowell et al., J Amer Chem Soc, 114:9245-9253 (1992); the teachings of which are incorporated herein by reference in their entirety. Such fragments may be fused to carrier molecules, such as for example, immunoglobulins, for many purposes, including increasing the valency of protein binding sites. For example, fragments of the protein may be fused through “linker” sequences to the Fc portion of an immunoglobulin. For a bivalent form of the protein, such a fusion could be to the Fc portion of an IgG molecule. Other immunoglobulin isotypes may also be used to generate such fusions. For example, a protein-IgM fusion would generate a decavalent form of the protein.

By “antibody” is meant an immunoglobulin, intact or a fragment thereof, that is capable of binding an epitopic determinant. Such antibodies may be produced utilizing the polypeptide sequences of the present invention according to methods described below. By “humanized antibody” is meant an antibody molecule in which the amino acid portion of the non-antigen binding region is modified to more closely resemble a human antibody amino acid sequence, while retaining its original ability to bind. Methods for producing such “humanized” molecules are generally well known and described in, for example, U.S. Pat. No.: 4,816,397.

By “associated gene” is meant a region of the genome that is transcribed to produce the mRNA from which each cDNA sequence is derived and may include contiguous regions of the genome necessary for the regulated expression of each gene. An associated gene may therefore include, but is not intended to be limited to, regions corresponding to coding sequences, 5′ and 3′ untranslated regions, alternatively spliced exons, introns, promoters, and silencer or suppressor elements.

By “binding partner” is meant a molecule that is capable of binding specifically to another molecule, such as for example, an antibody and its specific antigen, a receptor and its interacting hormone or an enzyme and an inhibitor.

By “biologically active” is meant having a naturally occurring function, that is either a structural function or a biochemical function. Biological activity includes antigenic activity.

By “cell adhesion-related” or “cell adhesion-mediated” (and grammatical variations thereof) is meant involvement in the establishment, maintenance or regulation of cell attachment either between cells or between cells and substrate molecules. By “cell adhesion-related disorder” or “cell adhesion-mediated disorder” (and grammatical variations thereof) is meant a condition or disease characterized by alterations in cell-cell adhesion or cell-substrate adhesion such as occurs for example, in cancer, especially metastatic cancer or endometriosis. Examples of cell adhesion mediated disorders or diseases include prostate cancer, breast cancer, lung cancer, colorectal cancer, muscular dystrophy, blistering diseases, inflammatory disease, atherosclerosis and developmental disorders. Cell adhesion-mediated disorders or diseases relate to cancers wherein, for example, cells from primary tumors metastasize to secondary sites, frequently showing a marked preference for particular tissues. For example, prostate cancer tends to metastasize to bone while colorectal cancer tends to metastasize to the liver.

By “chemical derivative” is meant a subject polypeptide having one or more residues chemically derivatized by a reaction of a functional side group. Such derivatized residues include for example, those molecules in which free amino acid groups have been derivatized to form amine hydrochlorides, p-toluene sulfonyl groups, carbobenzoxy groups, t-butyloxycarbonyl groups, chloroacetyl groups or formyl groups and the like. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to for N-imbenzylhistidine. Also included as chemical derivatives are those peptides that contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and omithine may be substituted for lysine. “Chemically derivatized” is meant to include tags such as for example, green fluorescent protein and hemagglutinin (HA).

By “coding sequence” is meant a polynucleotide sequence which is transcribed into mRNA and translated into a polypeptide when placed under the control of appropriate regulatory sequences. The boundaries of the coding sequence are determined by a translation start codon at the 5′-terminus and preferably, but not always, by a translation stop codon at the 3′-terminus. Such boundaries can be naturally occurring or can be introduced into or added to the polynucleotide sequence by methods known in the art. A coding sequence can include, but is not limited to, mRNA, cDNA, and recombinant polynucleotide sequence.

By “conservative amino acid substitution” is meant, an amino acid substitution that based upon the chemical structure and function of the polypeptide into which the substitutions are to be made, least affects the structure and function of the polypeptide. For example, if a beta sheet structure is present in the polypeptide before substitution, then a beta sheet structure would be preserved after substitution. For polypeptide sequences, such conservative substitutions consist of substitution of one amino acid at a given position for another amino acid of the same class (amino acids that share characteristic of hydrophobicity, charge, pK or other conformational or chemical properties, valine for leucine, arginine for lysine) or by one or more non-conservative amino acid substitutions; deletions or insertions, located at positions of the sequence that do not alter the conformation or folding of the polypeptide to the extent that the biological activity of the polypeptide is destroyed. The function of the original polypeptide is essentially preserved after such a substitution also. Conservative amino acid substitutions include substitutions of one non-polar (hydrophobic) residue such as isoleucine, valine, leucine or methionine for another; the substitution of one polar (hydrophilic) residue for another such as between arginine and lysine, between glutamine and asparagines, between threonine and serine; the substitution of one acidic residue, such as aspartic acid or glutamic acid for another; or the use of a chemically derivatized residue in place of a non-derivatized residue; provided that the polypeptide displays the requisite biological activity.

| | |
|---|---|
| Amino Acid | Conservative Substitute |

| | |
|---|---|
| Ala | Gly, Ser |
| Arg | His, Lys |
| Asn | Asp, Gln, His |
| Asp | Asn, Glu |
| Gln | Glu, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Trp | Phe, Tyr |
| Tyr | His, Phe, Tyr |
| Val | Ile, Leu, Thr |

By “detectable label” is meant a reporter moiety or enzyme that is attachable to a polynucleotide or polypeptide that is capable of generating a detectable signal. Examples of labels include radioactive tags, fluorescent tags, chemiluminescent tags, enzyme substrates that can be activated by an enzyme to thereby generate a signal.

By “fragment” of a protein of the present invention is meant any amino acid sequence shorter than that of the protein, comprising at least 6, preferably at least 10, more preferably at least 20, and most preferably at least 50 consecutive amino acids of the full polypeptide. Such molecules may or may not also comprise additional amino acids derived from the process of cloning, amino acid residues or sequences corresponding to full or partial linker sequences. Fragments include the polypeptides encoded by the exons of the present invention.

By “fragment” of a polynucleotide of the present invention is meant a unique portion of a polynucleotide of the present invention such as can be used for example, in a yeast two hybrid assay, as a probe, as a primer or as a therapeutic molecule. Such a fragment is identical to some portion of the original polynucleotide and is at least 6, 8, 10, 12, 15, 20, 25, 30, 50, 100, 200 or 500 nucleotides in length. Fragments include the nucleic acid sequences of the exons provided herein.

By “immune response” is meant a biological response of an animal, preferably a mammal, to an antigen that is characterized by the formation of antibodies and/or by inflammation and cytokine secretion such as for example, in response to trauma or disease.

By “immunogenic fragment” is meant a polypeptide or oligopeptide capable of eliciting an immune response.

By “mutant” of a nucleic acid sequence is meant a polynucleotide that includes any change in the nucleotide base sequence relative to a nucleotide sequence of the present invention. Such changes can arise either spontaneously or by manipulations by man, such as by radiation (i.e., x-ray) or by forms of chemical mutagenesis or by genetic engineering or as a result of mating or other forms of exchange of genetic information. Mutations include, for example, base changes, deletions, insertions, inversions, translocations or duplication in the nucleotide sequence. Mutant forms of the polynucleotide may affect cell-adhesion-mediated activity of a cell or tissue by affecting the stability of the polynucleotide transcript, the efficiency of its translation into polypeptide, the type or efficiency of production of splicing variants and may produce changes in the encoded polypeptide or such mutant changes may be silent. Such mutants may or may not also comprise additional nucleic acids derived from the process of cloning, nucleic acid residues or sequences corresponding to full or partial linker sequences. By “mutant” of a protein is meant a polypeptide that includes any change in the amino acid sequence relative to the amino acid sequence of a polypeptide sequence of the present invention. Mutant forms of the protein may affect cell adhesion-mediated activity of a cell or tissue or they may not. Activity is measured relative to the polypeptide of the present invention, and such mutants may or may not also comprise additional amino acids derived from the process of cloning, amino acid residues or sequences corresponding to full or partial linker sequences.

By “nucleic acid” or “polynucleotide” is meant a length of DNA or RNA produced by an organism or synthesized by any means (e.g., cell-free system; chemically) and may include coding regions, regulatory regions or other sequences. Nucleic acid, especially in the form of probes, includes peptide nucleic acid.

By “polypeptide,” “peptide” or “protein” is meant a chain of amino acids, regardless of length or post-translational modification (glycosylation or phosphorylation). These terms include naturally-occurring polypeptides and proteins, as well as those that are synthetic or recombinant.

By “probe” is meant an isolated nucleic acid or peptide nucleic acid sequence or fragment, and their complements, that are useful for detecting related nucleic acid sequences. Frequently a probe is labeled, such as for example, with an enzyme, a dye or a radioactive label. Such probes are useful in hybridization assays for determining the presence or absence of nucleic acid sequence. Methods of making and using probes and primers can be found for example, in Sambrook, J. et al., Molecular Cloning: A Laboratory Manual, 2nd edition, Cold Harbor Press Press, Plainview, N.Y., Vol. 1-3 (1989); Ausubel, F. M., et al., Current Protocols in Molecular Biology, Greene Publ. Assoc. & Wiley-Intersciences, New York, N.Y.; Innis, M. et al., PCR Protocols, A Guide to Methods and Applications, Academic Press, San Diego, Calif. (1990). PCR primer pairs can be derivatived using software such as for example, Primer3 (Whitehead Institute for Biomedical Research, Cambridge, Mass.); OLIGO ver. 4.06 PrimOU (Genome Center at the University of Texas Southwest Medical Center, Dallas, Tex.).

By “sequence homology” is meant both sequence identity and sequence similarity. “Sequence identity” or “sequence similarity” are relationships between two or more polynucleotide or polypeptides sequences and these relationships are determined by comparing the sequences. “Similarity” between two polypeptides is determined by evaluating the conserved amino acid substitutions between the two sequences. “Sequence identity,” as used herein, refers to the subunit sequence similarity between two polymeric molecules, e.g., two polynucleotides or two polypeptides. When a subunit position in both of the two molecules is occupied by the same monomeric subunit, if a position in each of two peptides is occupied by a serine, then they share sequence identity at that position. The identity between two sequences is a direct function of the number of matching or identical positions, if half (e.g., 5 positions in a polymer 10 subunits in length) of the positions in two peptide or compound sequences are identical, then the two sequences are 50% identical; if 90% of the positions are identical, e.g., 9 of 10 are matched then the two sequences share 90% sequence identity.

Identity is often measured using sequence analysis software, BLASTN or BLASTP (available on the world wide web at ncbi.nlm.nih.gov/BLAST/). The default parameters for comparing two sequences by BLASTN (for nucleotide sequences) are reward for match=1, penalty for mismatch=−2, open gap=5, and extension gap=2. When using BLASTP for protein sequences, the default parameters are reward for match=0, penalty for mismatch=0, open gap=11, and extension gap=1.

Sequence identity may also be determined using WU-BLAST (Washington University BLAST) version 2.0 software, which builds upon WU-BLAST version 1.4, which in turn is based upon the public domain NCBI-BLAST version 1.4 (Altschul and Gish, “Local alignment statistics,” Doolittle ed., Methods in Enzymology, 266:460-480 (1996); Atschul et al., “Basic local alignment search tool,” J of Molecular Biology, 215:403-410 (1990); Gish and States, “Identification of protein coding regions by database similarity search,” Nature Genetics, 3:266-272 (1993); Karlin and Altschul, “Applications and statistics for multiple high-scoring segments on molecular sequences,” Proc Natl Acad Sci USA, 90:5873-5877 (1993); each of which are incorporated herein by reference in its entirety). WU-BLAST version 2.0 executable programs for several UNIX platforms can be downloaded from ftp://blast.wustl.edu/blast/executables. The complete suite of search programs (BLASTN, BLASTP, BLASTX, TBLASTN, and TBLASTX) is provided at that site, in addition to several support programs. WU-BLAST version 2.0 is copyrighted and may not be sold or distributed in any form or manner without the express written consent of the author; but the posted executable programs may otherwise be used freely for commercial, nonprofit or academic purposes. In all programs in the suite—BLASTN, BLASTP, BLASTX, TBLASTN and TBLASTX—the gapped alignment routines are integral to the database itself, and thus yield much better sensitivity and selectivity while producing the more easily interpreted output. Gapping can optionally be turned off in all of these programs, if desired. The default penalty (Q) for a gap of length one is Q=9 for proteins and BLASTP and Q=10 for BLASTN, but may be changed to any integer value including zero, one through eight, nine, ten eleven, twelve through twenty, twenty-one through fifty, fifty-one through one hundred, etc. The default per residue penalty for extending a gap® is R=2 for proteins and BLASTP, and R=10 for BLASTN, but may be changed to any integer value including zero, one, two, three four, five, six, seven, eight, nine, ten, eleven, twelve, through twenty, twenty-one through fifty, fifty-one through one hundred, etc. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Protein sequences are compared to known sequences using protein sequence databanks, such as GenBank, Brookhaven Protein, SWISS-PROT and PIR, to determine potential sequence homologies. This information facilitates elimination of sequences that exhibit a high degree of sequence homology to other molecules, thereby enhancing the potential for high specificity in the development of antisera, agonists and antagonists to the proteins disclosed herein.

Homology for polypeptides is typically measured using sequence analysis software (Sequence Analysis Software Package of the Genetics Computer Group, University of Wisconsin Biotechnology Center, 1710 University Avenue, Madison, Wis. 53705). Protein analysis software matches similar sequences by assigning degrees of homology to various substitutions, deletions, and other modifications.

Species homologs of the disclosed polynucleotides and proteins are also provided by the present invention. As used herein, a “species homolog” is a protein or polynucleotide with a different species of origin from that of a given protein or polypeptide, but with significant sequence similarity to the given protein or polynucleotide. Preferably, polypeptide species homologs have at least 60% sequence identity (more preferably, at least 80% identity; most preferably at least 90% identity) with the given protein, where the sequence is determined by comparing the amino acid sequences of the proteins when aligned so as to maximize overlap and identity while minimizing sequence gaps. Species homologs may be isolated and identified by making suitable probes or primers from the polynucleotide sequences provided herein and by screening a suitable nucleic acid source from the desired species. Preferably, species homologs are those isolated from mammalian species. Most preferably, species homologs are those isolated from certain mammalian species such as, for example, Pan troglodytes, Gorilla gorilla, Pongo pygmaeus, Hylobates concolor, Macaca mulatta, Papio papio, Papio hamadryas, Cercopithecus aethiops, Cebus capucinus, Aotus trivirgatus, Sanguinus Oedipus, Microcebus murinus, Mus musculus, Rattus norvegicus, Cricetulus griseus, Felis catus, Mustela vison, Canis familiaris, Oryctolagus, Bos Taurus, Ovis arie, Sus scrofa and Equus caballus, for which genetic maps have been created allowing the identification of syntenic relationships between the genomic organization of genes in one species and the genomic organization of the related genes in another species (O'Brien and Seuanez, Ann Rev Genet, 22:323-351 (1988); O'Brien et al., Nature Genetics, 3:103-112 (1993); Johansson et al., Genomics, 25:682-690 (1995); Lyons et al., Nature Genetics, 15:47-56 (1997); O'Brien et al., Trends in Genetics, 13(10):393-399 (1997); Carver and Stubbs, Genomic Research, 7:1123-1137 (1997); each of which is incorporated herein in its entirety).

By “substantially purified” or “isolated” is meant an amino acid or nucleic acid that is removed from its natural environment and separated therefrom, and that is preferably at least 60%, more preferably 75% and most preferably 90% free from other components present in its natural environment.

By “variant,” is meant a polynucleotide (or polypeptide) that differs from a reference polynucleotide (or polypeptide), respectively. By “reference polynucleotide” is meant a polynucleotide of the present invention encoding a corresponding polypeptide of the present invention. A “variant” polynucleotide may be an “allelic” variant, a “splice” variant, a “species” variant or a “polymorphic” variant. Allelic variants may be isolated and identified by making suitable probes or primers from the sequences provided herein and screening a suitable nucleic acid source from individuals of the appropriate species.

The differences between the variant and reference polynucleotide may be silent, i.e., they may not result in changes in the amino acids encoded by the polynucleotide, and the resulting polypeptide will have the same amino acid sequences as the reference polypeptide. Alternatively, the differences between the variant and reference polynucleotide may result in alterations in the amino acid sequence of the encoded polypeptide. Such alternations may take the form of amino acid substitutions, insertions, deletions, additions, truncations and fusions in the variant polypeptide and such alterations may be present in combination.

A variant sequence may also be a fragment of a reference polynucleotide or reference polypeptide, where the difference is that the variant sequence contains an internal or terminal addition or deletion.

The difference may also consist of amino acid residues that are substituted with conserved or non-conserved amino acid residues in the variant polypeptide. A polynucleotide or polypeptide of the invention may be a naturally occurring allelic variant or it may be a variant that is not known to occur naturally.

The variant polynucleotides and polypeptides described herein, may be splice variants of known polynucleotides or polypeptides. By “splice variant” is meant an alternative RNA produced by processing after transcription from a gene. Differing sections of polynucleotide sequence are deleted from a transcribed RNA molecule or less commonly joining separately transcribed RNA molecules, and may result in several mRNAs produced from the same gene. A splice variant may have significant identity to a reference sequence, be it polynucleotide or polypeptide, but will generally encode polypeptides having altered amino acid sequences. The term “splice variant” is also used herein to denote a protein encoded by a splice variant of an mRNA transcribed from a gene. A splice variant may arise as a result of a lack of or the addition of one or more exons in the polynucleotide as compared to the reference polynucleotide.

Such variants may also arise from RNA editing that occurs after transcription and consists of conversion of one type of base to another or the addition or deletion of bases (reviews, Chester, A et al., Biochem Biophys Acta, 1494:1-13 (2000); Maas, S and Rich, A, Bioessays, 22:790-802 (2000); Hanrahan, C J et al., Ann N Y Acad Sci, 868:51-66 (1999)).

By “vector” is meant a carrier into which pieces of nucleic acid may be inserted or cloned, which carrier may function to transfer the pieces of nucleic acid into a host cell. Such a vector may bring about the replication and/or expression of the transferred nucleic acid pieces.

The cells may be transformed in order to propagate the nucleic acid constructs of the invention or may be transformed so as to express one or more of the novel polypeptide sequences encoded by the nucleic acid construct. Cells transformed with the nucleic acid provided herein may be used to express any of the polypeptides described herein, including fusion proteins, functional domains or antigenic determinants of such protein(s).

The transformed cells of the invention may be used in assays to identify proteins and/or other compounds which affect specific biochemical manifestations of cancer such as for example, uncontrolled cellular division or metastasis. Transformed cells may be used to identify compounds which interact with any of the polypeptides provide herein, and/or which modulate the function or effects of the polypeptides provided herein. Transformed cells may be used to identify the interactions in biochemical pathways of a protein sequence of the present invention, such protein sequences include SEQ ID NOs: 2, 3, 5, 55, 65, 67, 71 and 73 or the amino acid sequences of SEQ ID NOs: 7,9, 11, 13, 15, 17, 19, 21, 23, 25, 27, 29, 31, 33, 35, 37, 39, 41, 43, 45, 47, 49, 51, 53, 57, 59, 61, 63 and 69. Interacting protein or protein fragments can be identified using a two-hybrid assay, such as exemplified in U.S. Pat. Nos.: 5,283,173; 5,468,614; 5,667,973; and 5,925,523; Fields and Song, Nature, 340:245-246 (1989), the disclosure of each of which is incorporated herein in its entirety.

Transformed cells may also be implanted into hosts, including humans, for therapeutic or other reasons, for example, for localized expression of a protein. Preferred host cells for implantation include mammalian cells from neuronal, fibroblast, bone marrow and spleen cell cultures. Preferred host cells also include embryonic stem cells and germ line cells.

In a further embodiment, the present invention provides transgenic animal models for cancer research. Such animal models can be used to evaluate a therapeutic effect of a treatment such as for example, passive immunization against at least one of the proteins of SEQ ID NOs: 2, 3, 5, 55, 65, 67, 71 and 73 for cancer treatment or to localize cancerous cells or to determine stage dependent changes in normal or cancerous tissue. Tumor growth and the occurrence of secondary tumors can be monitored. Such animal models can also be used to monitor localized delivery of a cytotoxic agent or the like, when conjugated to a molecule that specifically binds a polypeptide sequence of the present invention. The animal may be essentially any mammal, including rats, mice, hamsters, guinea pigs, rabbits, dogs, cats, goats, sheep, pigs and non-human primates. In addition, invertebrate models, including nematodes and insects, may be used for certain applications. The animal models are produced by standard transgenic methods including microinjection, transfection or by other forms of transformation of embryonic stem cells, zygotes, gametes, and germ line cells (or other cells rendered pluripotent) with vectors including genomic or cDNA fragments, minigenes, exons, homologous recombination vectors, viral insertion vectors and the like of genes encoding the protein for example, of SEQ ID NOs: 2, 3, 5, 55, 65, 67, 71 and 73 or any nucleic acid encoding the exon sequences provided herein, 7, 9, 11 13, 15, 17, 19, 21, 23, 25, 27, 29, 31, 33, 35, 37, 39, 41, 45, 47, 49, 51, 53, 57, 59, 61, 63 and 69. Suitable vectors include, but are not limited to, vaccinia virus, adenovirus, adeno-associated virus, retrovirus, liposome transport, neuraltropic viruses, and Herpes simplex virus. Such vectors can be used to insert a sequence (“knock-in”) or to block expression of a sequence (“knock-out) using techniques well known in the art, such as exemplified by U.S. Pat. Nos.: 4,736,866; 6,139,833; and 6,204,061, the disclosure of each of which is incorporated herein its entirety.

The animal models may include transgenic sequences comprising or derived from the nucleic acid sequences of the present invention, including normal and mutant sequences, intronic, exonic and untranslated sequences, and sequences encoding subsets of the sequence such as functional domains. Three major types of animal models are provided. The first model includes animals in which a normal human cell adhesion-mediating gene has been recombinantly introduced into the genome of the animal as an additional gene, under the regulation of either an exogenous or an endogenous promoter element, and as either a minigene or a large genomic fragment; in which a normal human cell adhesion-mediating gene has been recombinantly substituted for one or both copies of the animal's cell adhesion-mediating gene such as for example, that encodes the protein of SEQ ID NO: 65 by homologous recombination or gene targeting; and/or in which one or both copies of one of the animal's homologous cell adhesion-mediating genes have been recombinantly “humanized” by the partial substitution of sequences encoding the human homolog by homologous recombination or gene targeting.

The second model includes animals in which a variant human cell adhesion-mediating gene has been recombinantly introduced into the genome of the animal as an additional gene, under the regulation of either an exogenous or an endogenous promoter element, and as either a minigene or a large genomic fragment; in which a variant human cell adhesion-mediating gene has been substituted, using recombinant methods, for one or both copies of the animal's homologous cell adhesion-mediating gene by homologous recombination or gene targeting; and/or in which one or both copies of one of the animal's homologous genes have been recombinantly “humanized” by the partial substitution of sequences encoding a variant human homolog by homologous recombination or gene targeting.

The third model includes “knock-out” animals in which one or both copies of one of the animal's cell adhesion-mediating genes have been partially or completely deleted by homologous recombination or by gene targeting such as with double stranded RNA or that have been inactivated by the insertion or substitution by homologous recombination or gene targeting of exogenous sequences. In preferred embodiments, a transgenic mouse model for a cell adhesion-mediated disorder or disease has a transgene encoding a normal human cell adhesion-mediating protein, a variant human or murine cell adhesion-mediating protein or a humanized normal or variant murine cell adhesion-mediating protein generated by homologous recombination or by gene targeting.

The desired change in gene expression can be achieved through the use of antisense polynucleotides or ribozymes that bind and/or cleave the mRNA transcribed from the gene (Albert and Morris, Trends Pharmacol Sci, 15:250-254 (1994); Lavarosky et al., Biochem Mol Med, 62:11-22 (1997); and Hampel, Prog Nucleic Acid Res Mol Biol, 58:1-39 (1998)). The desired change in gene expression can also be achieved through the use of double-stranded ribonucleotide molecules having some complementarity to the mRNA transcribed from the genetic sequence(s) of the present invention, where the double-stranded RNA construct interferes with the transcription, stability or expression of the endogenous mRNA (“RNA interference” or RNAi”; Fire et al., Nature, 391:806-811 (1998); Montgomery et al., Proc Nat Acad Sci USA,. 95:15502-15507 (1998); and Sharp, Genes Dev, 13:139-141 (1999)).

Partial or complete gene inactivation can also be accomplished through insertion of transposable elements (Plasterk, Bioassays, 14(9):629-63 (1992); Zwaal et al., Proc Natl Acad Sci USA, 90(16):7431-7435 (1993); Clark et al., Proc Natl Acad Sci USA, 91(2):719-722 (1994)) or through homologous recombination, preferably detected by positive/negative genetic selection strategies (Mansour et al., Nature, 336:348-352 (1988); U.S. Pat. Nos.: 5,464,764; 5,487,992; 5,627,059; 5,631,153; 5,614,396; 5,616,491; and 5,679,52; or through creation of dominant negative transgenes (Ray, et al., Genes Dev, 5(12A):2265-73 (1991); Metsaranta, et al., J Cell Biol, 1992 118(1):203-12 (1992); Levin et al., EMBO J, 12(4):1671-80 (1993); Werner et al., EMBO J, 12(7):2635-43 (1993)). Dominant negative transgenes result in production of modified forms of a protein that when added to a cell or organism that is also producing the normal protein can interfere with the functioning of the normal protein. These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Transgenic animals, cells, tissues or organs that have multiple copies of the gene(s) corresponding to the polynucleotide sequence(s) disclosed herein, preferably produced by transformation of cells and their progeny, are also provided. Transgenic animals that have modified genetic control regions that increase or reduce gene expression levels or that change temporal or spatial patterns of gene expression, are also provided (see European Patent No: 0 649 464 B1, incorporated herein by reference in its entirety). Such transgenic animals can also be used for large-scale production of the proteins described herein, in the milk of transgenic mammals, as is described in U.S. Pat. No.: 5,962,648.

Additionally, the present invention includes the use of the polynucleotide sequences provided herein as probes. Such probes are particularly useful for identifying cancer characterized by an over- or under-expressed polynucleotide sequence(s) that have sequence identity or would hybridize with SEQ ID NOs: 1, 4, 54, 64, 66, 70 or 72 or respective complements. Such probes may be labeled, such as for example, radioactively or enzymatically, by methods well known by those of skill in the art. The probes of the present invention may be used in microarrays, for localization of cancerous tissue when conjugated to a reporter, for imaging cancerous tissue when conjugated to a reporter or for delivery of conjugated cytotoxic chemicals to a cell. Microarrays find use as diagnostic tools when used in a hybridization assay to develop characteristic patterns of differentially expressed genes for a disease state.

The present invention also provides both full-length and mature forms of the disclosed proteins. The full-length form of such proteins is identified in the sequence listing by translation of the nucleotide sequence of each disclosed clone. The mature form(s) of such protein may be obtained by expression of the disclosed full-length polynucleotide in a suitable mammalian cell or other host cell and include glycosylation or other post-translational modification. The sequence(s) of the mature form(s) of the protein may also be determinable from the amino acid sequence of the full-length form. As CEA family members SEQ ID NOs: 2, 3, 5, 55, 65, 67, 71 and 73 can have activity as recognition sites involved in cell-cell and/or cell-substrate adhesion or as receptors, such as for example, for a growth factor. Proteins of the present invention can affect angiogenesis. As recognition site proteins, the amino acid sequences of the present invention are useful for localizing a cell expressing such protein to a specific tissue or cell type for stem cell or gene therapy applications. The proteins of the present invention are useful as markers for identifying a particular tissue or cell type. Such recognition sites or receptors may allow targeting of specific molecules to a defined cell type or tissue. The proteins and polypeptides of the present invention can be used to generate specific polyclonal or monoclonal antibodies using methods well known in the art.

Proteins and protein fragments of the present invention include proteins with amino acid sequence lengths that are at least 25% (more preferably at least 50% and most preferably at least 75%), of the length of a disclosed protein and have at least 60% sequence identity (more preferably, at least 80% identity; most preferably at least 90% or 95% identity), with that disclosed protein, where sequence identity is determined by comparing the amino acid sequences of proteins when aligned so as to maximize overlap and identity while minimizing sequence gaps. Also included in the present invention are the protein and protein fragments that contain a segment preferably comprising ten (10) or more (preferably 20 or more; most preferably 30 or more), contiguous amino acids that share at least 75% sequence identity (more preferably, at least 85% identity; most preferably at least 95% identity), with any such segment of any of the disclosed proteins.

The invention also encompasses allelic variants of the disclosed polynucleotides or proteins; that is naturally-occurring alternative forms of the isolated polynucleotides which also encode proteins which are identical or which have significantly similar sequences to those encoded by the disclosed polynucleotides. Preferably, allelic sequences have at least 60% sequence identity with the given polynucleotide; more preferably, at least 75% identity; most preferably, at least 90% identity, where sequence identity is determined by comparing the nucleotide sequences of the polynucleotides when aligned so as to maximize overlap and identity while minimizing sequence gaps. Allelic variants may be isolated and identified by making suitable probes or primers from the sequences provided herein and screening a suitable nucleic acid source from individuals of the appropriate species.

A number of types of cells may act as host cells for expression of the protein. Mammalian host cells include, for example, monkey COS cells, Chinese Hamster Ovary (CHO) cells, human kidney 293 cells, human epidermal A431 cells, human Colo2O5 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternately, it may be possible to produce the protein in lower eukaryotes such as yeast or in prokaryotes such as bacteria. Potentially suitable yeast strains include, for example, Saccharomyces cerevisiae, Schizosaccharomyces pombe, Kluyveromyces strains, Candida or any yeast capable of expressing heterologous protein. Potentially suitable bacterial strains include, for example, Escherichia coli, Bacillus subtilis, Salmonella typhimium or any bacterial strain capable of expressing heterologous protein. If the protein is made in yeast or bacteria, it may be necessary to modify the protein produced therein, for example, by phosphorylation or glycosylation of the appropriate sites, in order to obtain the functional protein.

The protein may also be produced by operably linking the isolated polynucleotide of the invention to a suitable control sequence in one or more insect expression vector, and employing an insect expression system. Materials and methods for baculovirus/insect cell expression systems are commercially available in kit form from, Invitrogen, San Diego, Calif., U.S.A. (the MaxBac® kit), and such methods are well known in the art, as described in Summers and Smith, 1987, Texas Agricultural Experiment Station Bulletin No. 1555, the disclosure of which is incorporated herein by reference in its entirety. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such cultures (i.e., from culture medium or cell extracts) using known purification processes, such as gel filtration and ion exchange chromatography. The purification of the protein may also include an affinity column containing agents which will bind to the protein; one or more column steps over such affinity resins as, for example, concanavalin A-agarose, heparin-toyopearl® or Cibacrom blue 3GA Sephroseg; one or more steps involving hydrophobic interaction chromatography using such resins as, for example, phenyl ether, butyl ether or propyl ether or immunoaffinity chromatography.

Alternatively, the protein of the invention may also be expressed in a form which will facilitate purification. For example, it may be expressed as a fusion protein, such as for example, those of maltose binding protein (MBP), glutathione-S-transferase (GST) or thioredoxin (TRX). Kits for expression and purification of such fusion proteins are commercially available form New England BioLabs (Beverly, Mass., U.S.A), Pharmacia (Piscataway, N.J., U.S.A.) and Invitrogen Corp. (Carlsbad, Calif., U.S.A.), respectively. The protein can also be tagged with an epitope and subsequently purified by using a specific antibody directed to such an epitope. One such epitope (also termed a “Flag”) is commercially available from Eastman Kodak Co. (New Haven, Conn. USA).

Finally, one or more reverse-phase high performance liquid chromatography (RP-HPLC) steps employing hydrophobic RP-HPLC media, silica gel having pendant methyl or other aliphatic groups, can be employed to further purify the protein. Some or all of the foregoing purification steps, in various combinations, can also be employed to provide a substantially homogenous isolated recombinant protein. The protein thus purified is substantially free of other mammalian proteins and is defined in accordance with the present invention as an “isolated protein.”

The protein of the present invention may be expressed as a product of transgenic animals, such as a component of milk of transgenic cows, goats, pigs or sheep which are characterized by somatic or germ cells containing a nucleotide sequence of the present invention encoding the protein. Such methods are described, in U.S. Pat. No.: 5,962,648, the disclosure of which is incorporated herein by reference in its entirety.

The protein of the present invention may also be expressed as a product of transgenic plants, as a component of a plant part such as the vegetative matter, fruit or seeds. Such plants and plant parts are characterized by somatic or germ cells containing a nucleotide sequence of the present invention encoding the protein. Such methods are described, for example, in U.S. Pat. Nos.: 5,990,358 and 5,994,628, the disclosure of each of which is incorporated herein by reference in its entirety.

The protein may also be produced by known conventional chemical synthesis. Methods for constructing the proteins of the present invention by synthetic means are known to those of skill in the art. The synthetically constructed protein sequences, by virtue of sharing primary, secondary or tertiary structural and/or conformational characteristics with proteins may possess biological properties common therewith, including protein activity. Thus, they may be employed as biologically active or immunological substitutes for natural, purified proteins in screening of therapeutic compounds and in immunological processes for the development of antibodies.

The proteins provided herein include proteins characterized by amino acid sequences similar to those of purified proteins but into which modifications are naturally provided or deliberately engineered. For example, modifications in the peptide or DNA sequences can be made by those of skill in the art using known techniques. Modifications of interest in the protein sequences may include alteration, substitution, replacement, insertion or deletion of a selected amino acid residue in the coding sequence. For example, one or more cysteine residues may be deleted or replaced with another amino acid to alter the conformation of the protein molecule. Techniques for such alteration, substitution, replacement, insertion or deletion are well known in the art (see for example, U.S. Pat. No.: 4,518,584, the disclosure of which is incorporated herein by reference in its entirety). In making such changes, substitutions of like amino acids residues can be made on the basis if relative similarity of side-chain substituents and properties, such as for example, size, charge, hydrophobicity, hydrophilicity and the like. Alterations of the type described may be made to enhance the potency or stability to enzymatic breakdown or pharmacokinetics of the polypeptide. It is well known that modifications and changes can be made without substantially altering the biological function of the polypeptide/protein and preferably such alternation, substitution, replacement, insertion or deletion retains the desired activity of the protein. Thus, sequences deemed within the scope of the present invention include those analogous sequences characterized by a change in amino acid sequence or type, wherein the change does not alter the fundamental nature and biological activity of the aforementioned proteins, derivatives, mutants, fragments and/or fusion proteins.

The present invention also describes fragments, mutants, analogs and species homologs of the proteins described herein. A fragment is any amino acid sequence shorter than that of the protein, comprising at least 6 consecutive amino acids of the full polypeptide. Such molecules may or may not also comprise additional amino acids derived from the process of cloning, amino acid residues or sequences corresponding to full or partial linker sequences. To be encompassed by the present invention, such mutants, with or without such additional amino acid residues, must have substantially the same biological activity as the natural or full-length version of the reference polypeptide. Mutant forms of the protein may display either increased or decreased cell adhesion enhancing activity relative to the equivalent reference polypeptide, and such mutants may or may not also comprise additional amino acids derived from the process of cloning, amino acid residues or sequences corresponding to full or partial linker sequences.

It is possible that a given polypeptide may be either a fragment, a mutant, an analog or an allelic variant of the protein or it may be two or more of those things, a polypeptide may be both an analog and a mutant of the polypeptide. For example, a shortened version of the molecule (a fragment of the protein) may be created in the laboratory. If that fragment is then mutated through means known in the art, a molecule is created, which is later discovered to exist as an allelic form of the protein in some mammalian individuals. Such a mutant molecule would therefore be both a mutant and an allelic variant. Such combinations of fragments, mutants, allelic variants and analogs are intended to be encompassed in the present invention.

The present invention also includes fusions proteins and chimeric proteins comprising the proteins, their fragments, mutants, species homologs, analogs and allelic variants. A fusion protein or chimeric protein can be produced as a result of recombinant expressions and the cloning process, for example, the protein may be produced comprising additional amino acids or amino acid sequences corresponding to full or partial linker sequences, the protein of the present invention, when produced in E. coli, can comprise additional vector sequence added to the protein, including a histidine tag. As used herein, the term “fusion protein” or “chimeric protein” is intended to encompass changes of this type to the original protein sequence. A fusion or chimeric protein can consist of a multimer of a single protein, repeats of the protein sequence or the fusion and chimeric proteins can be made up of several proteins. The fusion or chimeric protein can comprise a combination of two or more known proteins or a polypeptide-polynucleotide hybrid, such as for example, is used in a two-hybrid protein-protein interaction assay (Fields and Song, “A novel genetic system to detect protein-protein interactions,” Nature, 340:245-246 (1989), the disclosure of which is incorporated herein by reference in its entirety) or a protein in combination with an immunoglobulin molecule. The fusion or chimeric proteins can also include proteins, their fragments, mutants, species homologs, analogs and allelic variants, and other proteins, a reporter probe comprising a protein of interest and an enzyme capable of activating a substrate. The term “fusion protein” or “chimeric protein” as used herein can also encompass additional components, such as for example, for delivering a chemotherapeutic agent, wherein a polynucleotide encoding the therapeutic agent is linked to the polynucleotide encoding the protein. Fusion or chimeric proteins can also encompass multimers of a protein, dimers or trimers. Such fusion or chimeric proteins can be linked together via a post-translational modification such for example, a chemical linage or the entire fusion protein may be made recombinantly.

Multimeric proteins comprising the proteins disclosed herein, their fragments, mutants, species homologs, analogs and allelic variants are also meant to be encompassed by the present invention. By “multimer” is meant a protein sequence comprising two or more copies of a subunit protein. The subunit protein may be one of the proteins of the present invention, such as for example, the protein of SEQ ID NO: 65, repeated two or more times or a fragment, mutant, homolog, analog or allelic variant of, for example, SEQ ID NO: 65 mutant or fragment repeated two or more times or combinations thereof. Such a multimer may also be a fusion or chimeric protein, such as for example, a repeated SEQ ID NO: 65 mutant may be combined with a polylinker sequence, and one or more other peptides, which may be present in single copy or may be tandemly repeated, a protein may comprise two or more multimers with in the overall protein.

The present invention also encompasses a composition comprising one or more of the isolated polynucleotide(s) encoding the protein(s) described herein, as well as vectors and host cells containing such a polynucleotide, and processes for producing the proteins, and their fragments, mutants, species homologs, analogs and allelic variants. The term “vector” as used herein means a carrier into which pieces of nucleic acid may be inserted or cloned, which carrier functions to transfer the pieces of nucleic acid into a host cell. Such a vector may also bring about the replication and/or expression of the transferred nucleic acid pieces. Examples of vectors include nucleic acid molecules derived from, for example, a plasmid; a bacteriophage; a mammalian, plant or insect virus; or non-viral vectors such as ligand-nucleic acid conjugates, liposomes or lipid-nucleic acid complexes. It may be desirable that the transferred nucleic acid molecules is operably linked to an expression control sequence to form an expression vector capable of expressing the transferred nucleic acid.

The vector into which the polynucleotide is cloned may be chosen because it functions in a prokaryotic or alternatively, in a eukaryotic organism. Two examples of vectors which allow for both the cloning of a polynucleotide encoding a protein, and the expression of that protein from the polynucleotide, are the pET22b and the pET28 (a) vectors (Novagen, Madison, Wis., USA) and a modified pPICZaA vector (In Vitrogen, San Diego, Calif., USA) which allow expression of the protein in bacteria and yeast, respectively. See for example, WO 99/29878, the entire teachings of which are hereby incorporated herein by reference.

In one embodiment, the isolated polynucleotide encoding the protein additionally comprises a polynucleotide linker encoding a protein. Such linkers are known to those of skill in the art and, for example, the linker can comprise at least one additional codon encoding at least one additional amino acid. Typically the linker comprises one to about twenty or thirty amino acids. The polynucleotide is translated, as is the polynucleotide encoding the protein, resulting in the expression of a protein with at least one additional amino acid residue at the amino or carboxyl terminus of the protein. Importantly, the additional amino acid or amino acids, do not compromise the activity of the protein.

After inserting the selected polynucleotide onto the vector, the vector is transformed into an appropriate prokaryotic (or eukaryotic) strain and the strain is cultured (e.g., maintained) under suitable conditions for the production of the biologically active protein, thereby producing a biologically active protein or mutant, derivative, fragment or fusion protein thereof. For example, a polynucleotide encoding a protein can be cloned into a vector such as for example, pET22b, pET17b or pET28a, which is then transformed into bacteria. The bacterial host strain then expressed the protein, under appropriate conditions. With such vectors, the proteins are typically produced in quantities of about 10-20 m/g or more per L of culture fluid.

The eukaryotic vector can comprise a modified yeast vector. One method is to use a pPICZ plasmid, wherein the plasmid contains a multiple cloning site. The multiple cloning site has inserted into the multiple cloning site a His. Tag motif. Additionally, the vector can be modified to add a NdeI site or other suitable restriction sites. Such sites are well known to those of skill in the art. Proteins produced by this embodiment comprise a histidine tag motif (His. Tag) comprising one or more histidines, typically about 5-20 histidines. The tag must not interfere with the properties of the protein.

One method of producing the proteins described herein is, for example, to amplify the polynucleotide of SEQ ID NO: 64, and clone it into an expression vector, pET22b, pET28(a), pPICZ A or some other expression vector, transform the vector containing the polynucleotide into a host cell capable of expressing the polypeptide encoded by the polynucleotide, culturing the transformed host cell under culture conditions suitable for expressing the protein, and then extracting and purifying the protein from culture. The protein may be expressed as a product of transgenic animals, such as for example, as a component of the milk of cows, goats, sheep or pigs or as a product of a transgenic plant, such as for example, combined or linked with starch molecules in maize. These methods can also be used with subsequences of SEQ ID NO: 1 to produce portions of the protein of SEQ ID NOs: 2, 3 or 4 to produce portions of the protein of SEQ ID NOs: 5 or 54 to produce SEQ ID NOs: 55 or 64 to produce SEQ ID NOs: 65 or 66 to produce SEQ ID NOs: 67 or 70 to produce SEQ ID NOs: 71 or 72 to produce SEQ ID NO: 73.

The polynucleotides and proteins of the present invention can also be used to design probes to isolate other proteins and gents encoding the proteins that are species homologs or have the same or similar properties. Exemplary methods are provided in U.S. Pat. No.: 5,837,490, by Jacobs et al., the disclosure of which is herein incorporated by reference in its entirety. The design of an oligonucleotide probe should preferably follow these parameters: a) it should be designed to an area of the sequence which has the fewest ambiguous bases (“N's”), if any; and b) it should be designed to have a Tm of approximately 80° C. (assuming 2° C. for each “A” or “T” and 4° for each “G” or “C”).

The oligonucleotide should preferably be labeled such as for example, with g-32P-ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, the specific activity of the resulting probe should be approximately 4×106 dpm/pmole. The bacterial culture containing the pool of full-length clones should preferably be thawed and 100 l of the stock used to inoculate a sterile culture flask containing 25 ml of sterile L-broth containing ampillicin at 100 l/ml. The culture should preferably be grown to saturation at 37° C., and the saturated culture should preferably be diluted with in fresh L-broth. Aliquotes of these dilutions should preferably be plated to determine the dilution and volume which will yield approximately 5000 distinct and well-separated colonies on solid bacteriological media containing L-broth containing ampicillin at 100 l/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters for identification of clones containing nucleic acid of interest (“positive clones”) through the use of at least one probe. The colonies on the filter should be lysed; the genetic material denatured; and the resultant material baked on the filter. The probe should be chosen for use based upon its ability to bind the nucleic acid sequence(s) in interest on the filter when using the selected stringency conditions.

The filter is preferably incubated at 65° C. for 1 hour with gentle agitation in 6×SSC (20×stock is 175.3 g NaCl/liter, 88.2 g Na citrate/liter, adjusted to pH 7.0 with NaOH) containing 0.5% SDS (sodium dodecyl sulfate), 100 mg/ml of yeast RNA, and 10 mM EDTA (approximately 10 mL per 150 mm filter). Preferably, the probe is then added to the hybridization mix at a concentration greater than or equal to 1×106 dpm/mL. The filter is then preferably incubated at 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at 65° C. for 30 mins. to 1 hour is optional. The filter is then preferably dried and subjected to autoradiography for sufficient time to visualize the positives on the X-ray film. Other known hybridization methods can also be employed.

Stringency conditions for hybridization refer to conditions of temperature and buffer composition which permit hybridization of a first nucleic acid sequence to a second nucleic acid sequence, wherein the conditions determine the degree of identity required between those sequences which hybridize to each other. Preferably, there is at least 70% identity between such sequences, more preferably at least 90% and most preferably at least 95% identity. Therefore, “high stringency conditions” are those conditions wherein only nucleic acids sequences that are at least 95% similar to each other will hybridize. The sequences may be at least 90% similar to each other and still hybridize under moderate stringency conditions. When the nucleic acid sequences are even less similar they may hybridize to each other when low stringency conditions are used. By varying the washing conditions from a stringency level at which no hybridization occurs to a level at which hybridization is first observed, conditions for hybridization at which a known sequence will bind to an unknown sequence having a sequence most similar to the known sequence can be determined. The precise conditions determining the stringency of a particular hybridization include not only the ionic strength, temperature, and the condition of destabilizing agents such as formamide, but also on factors such as the length of the nucleic acid sequence, their base pair composition, the percent of mismatched base pairs between the two sequences, and the frequency of occurrence of subsets of the sequence(s) (small stretches of repeated sequences) within the unknown sequence. Washing is a step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature Tm for any chosen hybridization buffer (SSC) concentration. Generally, a doubling of the concentration of the SSC results in an increase in the Tm of about 17° C. Using these guidelines, the washing temperature can be determined empirically, depending upon the level of mismatch sought. Hybridization and wash conditions are explained in Current Protocols in Molecular Biology (Ausubel, F. M. et al., eds., John Wiley & Sons, Inc. (1995)), with supplemental updates on pages 2.10.1 to 2.10.16 and 6.3.1 to 6.3.6.

High stringency conditions that can be employed for hybridization include: (1) 1×SSC (10×stock at 3 M NaCl, 0.3 M Na3-citrate 2H2O (88 g/L), pH to 7.0 with 1 M HCl), 1% SDS, 0.1-2.0 mg/ml denatured salmon sperm DNA at 65° C.; (2) 1×SSC, 50% formamide, 1% SDS, 0.1-2.0 mg/ml denatured salmon sperm DNA at 42° C.; (3) 1% BSA (bovine serum albumen, fraction V), 1 mM Na2EDTA, 0.5 M NaHPO4 at pH 7.2 (1 M NaHPO4=134 g Na2HPO4. 7 H2O, 4 ml 85% H3PO4 per L), 7% SDS, 0.1-2.0 mg/ml denatured salmon sperm DNA at 65° C.; (4) 50% formamide, 5×SSC, 0.02 M Tris-HCl (pH 7.6), 1×Denhardt's solution (100×approx.=10 g Ficoll 400, 10 g polyvinylpyrrolidone, 10 g BSA (fraction 5), water 500 ml), 10% dextran sulfate, 1% SDS, 0.1-2.0 mg/ml denatured salmon sperm DNA at 42° C., (5) 5×SSC, 5×Denhardt's solution, 1% SDS, 100 mg/ml denatured salmon sperm DNA at 65° C.; (6) 5×SSC, 5×Denhardt's solution, 50% formamide, 1% SDS, 100 mg/ml denatured salmon sperm DNA at 42° C., with high stringency washes of either (1) 0.3-1×SSC, 0.1% SDS at 65° C.; or (2) 1 mM Na2EDTA, 40 mM Na2HPO4 (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tm of the hybrid, where Tm in ° C.=(2× the number of A and T bases)+(4× the number of G and C bases). For hybrids believed to be about 18 to 49 base pairs in length, the Tm in ° C.=(81.5° C.+16.6(log10M)+0.41(% G+C)−0.61(% formamide)−500×L), where “M” is the molarity of monovalent cations (Na+), and “L” is the length of the length of the hybrid in base pairs.

Moderate stringency conditions can employ hybridization at either (1) 4×SSC, pH to 7.0 with 1 M HCl, 1% SDS, 0.1-2.0 mg/ml denatured salmon sperm DNA at 65° C., (2) 4×SSC, 50% formamide, 1% SDS, 0.1-2.0 mg/ml denatured salmon sperm DNA at 42° C., (3) 1% BSA (fraction V), 1 mM Na2EDTA, 0.5 M Na2 HP04 (pH 7.2), 7% SDS 0.1-2.0 mg/ml denatured salmon sperm DNA at 65° C., (4) 50% formamide, 5×SSC, 0.02 M Tris-HCl (pH 7.6), 1×Denhardt's solution, 10% dextran sulfate, 1% SDS, 0.1-2.0 mg/ml denatured salmon sperm DNA at 42° C., (5) 5×SSC, 5×Denhardt's solution, 1% SDS, 100 mg/ml denatured salmon sperm DNA at 65° C.; or (6) 5×SSC, 5×Denhardt's solution, 50% formamide, 1% SDS, 100 mg/ml denatured salmon sperm DNA at 65° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tm of the hybrid, where Tm in ° C.=(2× the number of A and T bases)+(4× the number of G and C bases). For hybrids believed to be about 18 to 49 base pairs in length, the Tm in C=(81.5° C.+16.6(log10M)+0.41(% G+C)—0.61(% formamide)−500×L), where “M” is the molarity of monovalent cations (Na+), and “L” is the length of the length of the hybrid in base pairs.

Low stringency conditions can employ hybridization at either (1) 4×SSC, pH to 7.0 with 1 M HCl, 1%SDS, 0.1-2.0 mg/ml denatured salmon sperm DNA at 50° C., (2) 6×SSC, 50% formamide, 1% SDS, 0.1-2.0 mg/ml denatured salmon sperm DNA at 40° C., (3) 1% BSA (fraction V), 1 mM Na2EDTA, 0.5 M Na2HP04 (pH 7.2), 7% SDS, 0.1-2.0 mg/ml denatured salmon sperm DNA at 50° C., (4) 50% formamide, 5×SSC, 0.02 M Tris-HCl (pH 7.6), 1×Denhardt's solution, 10% dextran sulfate, 1% SDS, 0.1-2.0 mg/ml denatured salmon sperm DNA at 40° C., (5) 5×SSC, 5×Denhardt's solution, 1% SDS, 100 mg/ml denatured salmon sperm DNA at 50° C. or (6) 5×SSC, 5×Denhardt's solution, 50% formamide, 1% SDS, 100 mg/ml denatured salmon sperm DNA at 40° C., with low stringency washes of either 2×SSC,0.1% SDS at 50 can employ hybridization at either (1) 4×SSC, pH to 7.0 with 1 M HCl, 1%SDS, 0.1-2.0 mg/ml denatured salmon sperm DNA at 65° C., (2) 4×SSC, 50% formamide, 1% SDS, 0.1-2.0 mg/ml denatured salmon sperm DNA at 42° C., (3) 1% BSA (fraction V), 1 mM Na2EDTA, 0.5 M Na2 HPO4 (pH 7.2), 7% SDS, 0.1-2.0 mg/ml denatured salmon sperm DNA at 50° C., (4) 50% formamide, 5×SSC, 0.02 M Tris-HCl (pH 7.6), 1×Denhardt's solution, 10% dextran sulfate, 1% SDS, 0.1-2.0 mg/ml denatured salmon sperm DNA at 40° C., (5) 5×SSC, 5×Denhardt's solution, 1% SDS, 100 mg/ml denatured salmon sperm DNA at 50° C. or (6) 5×SSC, 5×Denhardt's solution, 50% formamide, 1% SDS, 100 mg/ml denatured salmon sperm DNA at 40° C., with low stringency washes of (1)1×SSC, 0.1% SDS at 50° C. or (2) 0.5% BSA (fraction V), 1 mM Na2EDTA, 40 mM Na2HPO4 (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tm of the hybrid, where Tm in ° C.=(2× the number of A and T bases)+(4× the number of G and C bases). For hybrids believed to be about 18 to 49 base pairs in length, the Tm in ° C.=(81.5° C.+16.6(log10M)+0.41(%G+C)−0.61(% formamide)−500×L), where “M” is the molarity of monovalent cations (Na+), and “L” is the length of the length of the hybrid in base pairs.

The present invention includes methods of diagnosing, treating and/ or preventing cell adhesion-mediated disease symptoms using the proteins described herein or their biologically active fragments, analogs, species homologs, derivatives or mutants. In particular, the present invention includes methods of treating a patient having a solid tumor such as for example, of the prostate, breast or colon with an effective amount of one or more of the proteins or with one or more of the biologically active fragments thereof or combinations of fragments that possess tumor growth modulating activity or with agonists thereof. An effective amount of protein is an amount sufficient either to inhibit metastasis or to induce apoptosis in cells involved in a disease or condition characterized by undesired or unchecked tumor growth, thus completely or partially alleviating the disease or condition. Alleviation of the cell adhesion-mediated disease can be determined by observing the symptoms of the disease, solid tumor growth or regression and/or metastasis of tumor cells and/or angiogenesis at the tumor site. As used herein, the term “effective amount” also means the total amount of each active component of the composition or method that is sufficient to show a meaningful patient benefit, e.g., treatment, healing, prevention or amelioration of such conditions. When applied to a combination, the term refers to combined amounts of the active ingredients that result in the therapeutic effect, whether administered in combination, serially or simultaneously. Cell adhesion-mediated diseases include, but are not limited to cancers, solid tumors, tumor metastasis, benign tumors (e.g., hemangiomas, acoustic neuromas, neurofibrous, organ fibrosis, trachomas, and pyogenic granulomas), muscular dystrophy, blistering diseases, inflammatory diseases, atherosclerosis, developmental disorders and endometriosis. “Regression” refers to the reduction of tumor mass and size as determined using methods well-known to those of skill in the art.

The antagonists or blockers of the cell adhesion-mediating activity of the proteins of the present invention may be used in combination with other compositions and procedures for treatment of disease. For example, a tumor may be treated conventionally with surgery, radiation, chemotherapy or immunotherapy, and then an antagonist or antibody to a protein of the present invention may be administered to the patient to extend the dormancy of the micrometastases and to stabilize and inhibit the growth of any residual primary tumor. The antisesra or antagonists to the inventive proteins or fragments or combinations thereof, can also be combined with other cancer-modulating compounds or proteins, fragments, antisera, receptor agonists, receptor antagonists of other cancer-modulating proteins. Additionally, the antisera and/or receptor antagonists or combinations thereof, may be combined with pharmaceutically acceptable excipients, and optionally sustained release matrix such as biodegradable polymers, to form therapeutic compositions. The compositions of the present invention may also contain apoptosis-modulating proteins or chemical compounds, and mutants, fragments and analogs thereof. Such additional factors and/or agents may be included in the compositions to minimize side effects. Additionally, the composition of the present invention may be administered concurrently with other therapies, administration in conjunction with a chemotherapy or radiation regiment.

The invention includes methods for modulating cell-cell or cell-matrix adhesion in mammalian (e.g., human) tissues by contacting the tissue with a composition comprising the proteins or of a source of the proteins of the invention. Use of timed release or sustained release delivery systems are also included in the invention. Such systems are highly desirable where surgery is difficult or impossible, patient is debilitated by old age or disease or the course of treatment itself or where the risk-benefit analysis dictates control over cure.

A sustained-release matrix, as used herein, is a matrix made of materials, usually polymers, that are degradable by enzymatic or acid/base hydrolysis or by dissolution. Once inserted into the body, the matrix is acted upon by the enzymes and body fluids. The sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is one of polylactide, polyglycolide or polylactide co-glycolide.

The cell adhesion-mediating composition of the present invention may be a solid, a liquid or an aerosol and may be administered by any known route of administration. Examples of solid compositions include pills, creams, and implantable dosage units. The pills may be administered orally. The therapeutic creams maybe applied topically. The implantable dosage unit may be administered locally, for example, at the site of a solid tumor or may be implanted for systemic release, such as for example, subcutaneously. Examples liquid compositions include formulations adapted for injection subcutaneously, intravenously, intraarterially, and formulations for topic and intraocular administration. Examples of aerosol formulations include those adapted for use with an inhaler for administration to the lungs.

The proteins and protein fragments having cell adhesion-mediating activity described above can be provided as isolated and substantially purified proteins and protein fragments in pharmacologically acceptable formulations using formulation methods well known to those of skill in the art. These formulations can be administered by standard routes. In general, the combinations may be administered by topical, transdermal, intraperitoneal, intracranial, intracerebroventricular, intracerbral, intravaginal, intrauterine, oral, rectal or parenterally (intravenous, intraspinal, subcutaneous or intramuscular) route. In addition, the cell adhesion mediating proteins may be incorporated into biodegradable polymers allowing for sustained release of the compound, the polymers being implanted in the vicinity of where drug delivery is desired such as for example, proximal to a tumor of the prostate gland so that slow, sustained systemic delivery is achieved. Osmotic minipumps may also be used to provide controlled delivery of high concentrations of a protein of SEQ ID NOs: 2, 3, 5, 55, 65, 67, 71 or 73 through a cannula to the site of interest, such to the vascular surrounding the solid tumor or to the solid tumor itself. The biodegradable polymers and their use are described, for example, in Brem et al., J. Neurosurg., 74:441-446 (1991), which is hereby incorporated by reference in its entirety for what it teaches.

Modes of administration of the compositions of the present invention include intravenous, intramuscular, intraperitoneal, intrastemal, subcutaneous, and intraarticular injection and infusion. Pharmaceutical compositions for parenteral injection comprise pharmaceutically acceptable sterile aqueous or nonaqueous solutions, dispersions, suspensions or emulsions as well as sterile powders for reconstitution of sterile injectable solutions or dispersions just prior to use. Examples of suitable aqueous and nonaqueous carriers, diluents, solvents or vehicles include water, ethanol, polyois (, glycerol, propylene glycol, polyethylene glycol and the like, carboxymethylcellulose and suitable mixtures thereof, vegetable oils (, olive oil) and injectable organic esters such as ethyl oelate. Proper fluidity may be maintained for example, by use of coating materials such as lecithin, by the maintenance of the required particle size in the case of dispersions and by the use of surfactants. These compositions may also contain adjuvants such as preservatives, wetting agents, emulsifying agents and dispensing agents. Prevention of the action of microorganisms may be ensured by the inclusion of various antibacterial; and antifungal agents such as paraben, chlorobutanol, phenol sorbic acid, and the like. It may be desirable to include isotonic agents, such as sugar, sodium chloride, and the like. Prolonged absorption of the injectable pharmaceutical form may be brought about by the inclusion of agents such as aluminum monostearate and gelatin, which delay absorption. Injectable depot forms are made by forming microencapsulated matrices of the inventive composition in biodegradable polymers such as polylactide-polyglycolide, poly(orthoesters) and poly(anhydrides). Depending upon the ratio of inventive protein or polypeptide or the like to polymer and the nature of the particular polymer employed, the rate of release can be controlled. Depot injectable formulations are also prepared by entrapping the drug in liposomes or microemulsions that are compatible with the body tissues. The injectable formulation may be sterilized, for example, by filtration through a bacteria-retaining filter or by incorporating sterilizing agents in the form of sterile solid compositions that can be dissolved or dispersed in sterile water or other sterile injectable media just prior to use.

The therapeutic compositions of the present invention can include pharmaceutically acceptable salts of the components therein, that may be derived from inorganic or organic acids. By “pharmaceutically acceptable salt” is meant those salts that are, within the scope of sound medical judgment, suitable for use in contact with the tissues of humans and lower animals without undue toxicity, irritation, allergic response and the like are well-known in the art. For example, S. M. Berge, et al., J Pharmaceutical Sci, 66:1 et seq., (1977), which is incorporated herein by reference, describe pharmaceutically acceptable salts in detail. Pharmaceutically acceptable salts include the acid addition salts (formed with the free amino groups of the polypeptide) that are formed with inorganic acids such as for example, hydrochloric or phosphoric acids or such organic acids as acetic, tartaric, mandelic, and the like. Salts formed with the free carboxyl groups can be derived from inorganic bases such as for example, sodium, potassium, ammonium, calcium or ferric hydroxides, and such organic bases as isopropylamine, trimethylamine, 2-ethyl amino ethanol, histidine, procaine and the like. The salts may be prepared in situ during the final isolation and purification of the compounds of the invention or separately by reacting a free base function with a suitable organic acid. Representative acid addition salts include, but are not intended to be limited to, acetate, adipate, alginate, citrate, aspartate, benzoate, benezenesulfonate, bisulfonate, byutyrate, camphorate, camphorsulfonate, digluconate, glycerophosphate, hemisulfonate, heptonoate, hexanoate, fumarate, hydrochloride, hydrobromide, hydroiodide, 2-methanesulfonate (isethionate, lactate, maleate, methanesulfonate, nicotinate, 2-naphthalenesulfonate, oxalate, palmoate, pectinate, persulfate, 3-phenylpropionate, picrate, pivalate, propionae, succinate, tartate, thiocyanate, phosphate, glutamate, bicarbonate, p-toluenesulfonate, and undecanoate. Also, the basic nitrogen-containing groups can be quatemized with such agents as lower alkyl halides such as methyl, ethyl, propyl, and butyl chlorides, bromides, and iodides; dialkyl sulfates such as dimethyl, dibutyl, diamyl sulfates; long chain halides such as decyl, lauryl, myristyl and stearyl chlorides, bromides, and iodides; aralkyl halides such as benzyl and phenethyl bromides and others. Water or oil soluble or dispersible products are thereby obtained. Examples of acids that may be employed to form pharmaceutically acceptable acid addition salts include such inorganic acids as hydrochloric acid, hydrobromic acid, sulfuric acid, and phosphoric acid and such organic acids as oxalic acid, maleic acid, succininc acid and citric acid.

The active ingredient can be mixed with excipients that are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents, and the like which enhance the effectiveness of or enhance the stability of the active ingredient.

The dosage of the protein or fragment of the protein of the present invention will depend upon the disease state or condition being treated and other clinical factors such as the weight and condition of the human or animal and the route of administration of the compound. Depending upon the half-life of the protein in the particular animal or human, the protein can be administered between several times per day to once per week. It is to be understood that the present invention has application for both human and veterinary use. The methods of the present invention contemplate single as well as multiple administrations, given either simultaneously or over an extended period of time. In addition, the protein can be administered in conjunction with other forms of therapy, chemotherapy, immunotherapy or radiotherapy. In combination therapies, it may be possible to reduce the dosage of the inventive protein or polypeptide. For example, when tumor growth is being monitored, the dosage may vary with time depending upon the results of that monitoring.

The formulations of the present invention include those suitable for oral, rectal, ophthalamic (including intravitreal or intracameral), nasal, topical (including buccal and sublingual, intrauterine, vaginal or parenteral (including subcutaneous, intraperitoneal, intramuscular, intravenous, intraarterial, intradermal, intracranial, intratracheal, and epidural) administration. The formulations may be conveniently presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions that may contain anti-oxidants, buffers, bacteriostats and solutes that render the formulation isotonic with the blood of the intended recipient; and aqueous and non-aqueous sterile suspensions that may include suspending agents and thickening agents. The formulations may be presented in unit-dose or in multi-dose containers, for example, sealed ampules and vials, and may be stored in a freeze-dried (lyophilized) condition requiring only the addition of sterile liquid carrier, for example, water for injections, immediately prior to use. Extemporaneous injection solutions and suspensions may be prepared from sterile powders, granules, and tablets of the kind previously described.

When an effective amount of a protein or an antagonist of a protein of the present invention is administered orally, the protein(s) will be in a form of a tablet, capsule, powder, solution or elixir. When administered in tablet form, the pharmaceutical composition of the invention may additionally contain a solid carrier such as gelatin or an adjuvant. The tablet, capsule, and powder contain from about 5 to about 95% protein of the present invention, and preferably from about 25% to about 90% protein of the present invention. When administered in liquid form, a carrier such as water, petroleum oil, oils of animal or plant origin such as peanut oil, mineral oil, soybean oil or sesame oil or synthetic oil may be used. The liquid form of the pharmaceutical composition may further contain physiological saline solution, dextrose or other saccharide solution or glycols such as ethylene glycol, propylene glycol or poly ethylene glycol. When administered in liquid form, the pharmaceutical composition contains from about 0.5% to about 90% by weight of the protein of the present invention, and preferably from about 1 to 50% protein of the present invention.

When an effective amount of protein of the present invention is administered by intravenous, cutaneous or subcutaneous injection, protein of the present invention will be in the form of a pyrogen-free, parenterally acceptable protein solution. The preparation of such parenterally acceptable protein solutions, having due regard to pH, isotonicity, stability, and the like, is with in the skill of the art. A preferred pharmacological composition for intravenous, cutaneous or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection or other vehicle as known in the art. The pharmaceutical composition of the present invention may also contain stabilizers, preservatives, buffers, antioxidants or other additives known to those of skill in the art.

The amount of protein of the present invention in the pharmaceutical composition of the present invention will depend upon the nature and severity of the condition being treated, on the nature of prior treatments that the patient has undergone, and on the weight and condition of the patient. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further.

The duration of intravenous therapy using the pharmaceutical composition of the present invention will vary, depending upon the severity of the disease being treated and the condition and potential idiosyncratic response of each individual patient. It is contemplated that the duration of each application of the protein of the present invention will be in the range of 12 to 24 hours of continuous intravenous administration. Ultimately, the attending physician will decide on the appropriate duration of intravenous therapy using the pharmaceutical composition of the present invention.

Preferred unit dosage formulations are those containing a daily dose or unit, daily subdose or an appropriate fraction thereof, of the administered ingredient. It should be understood that in addition to the ingredients, particularly mentioned above, the formulations of the present invention may include other agents conventional in the art having regard to the type of formulation in question. Optionally, cytotoxic agents may be incorporated or otherwise combined with the cell adhesion-mediating proteins or biologically functional protein fragments thereof, to provide dual therapy to the patient.

The therapeutic compositions are also presently valuable for veterinary applications. Particularly domestic animals and thoroughbred horses, in addition to humans, are desired patients for treatment for cell adhesion-mediated disease or disorder with proteins of the present invention.

Cytotoxic agents such as ricin, can be linked to ligands and binding partners, such as for example, antibodies directed to the transmembrane cell adhesion-mediating proteins of the present invention, and fragments thereof, thereby providing a tool for the destruction of cells that bind or take-up such ligands or binding partners. These cells may be found in many locations, including but not limited to, micrometastases and primary tumors. A binding partner or a ligand are conjugated to a cytotoxic agent are infused in a manner designed to maximize delivery to a desired location. For example, ricin-linked high affinity antibodies are delivered through a cannula into vessels supplying the target site or directly into the target. Such agents are also delivered in a controlled manner through osmotic pumps coupled to infusion cannulae. A combination of agonists to the ligands of the cell adhesion-mediating protein may be co-applied with stimulators of apoptosis. This therapeutic regimen provides an effective means of destroying metastatic cancer.

Additional treatment methods include the administration of the cell adhesion-mediating protein(s), fragment(s), analog(s), antisera or receptor agonist(s) or antagonist(s) or binding partners thereof, linked to the cytotoxic agents, such as are well-known in the art and exemplified in WO0107476. It is to be understood that the cell adhesion-mediating protein(s) can be of human or of animal origin. The cell adhesion-mediating proteins can also be produced synthetically by chemical reaction or by recombinant techniques in conjunction with an expression system.

The present invention also encompasses the use of gene therapy or gene delivery to a host, whereby a polynucleotide of the present invention encoding a cell adhesion-mediating protein(s) of SEQ ID NOs: 1, 4, 54, 64, 66, 70 or 72 or a mutant, fragment or fusion protein thereof, such as one selected from the exons of SEQ ID NOs: 6, 8, 10, 12, 14, 16, 18, 20, 22, 24, 26, 28, 30, 32, 34, 36, 38, 40, 42, 44, 46, 48, 50, 52, 54, 56, 58, 60, 62 and 68 is introduced in a patient. Various methods of transferring or delivering the DNA to cells for expression of the gene product protein, otherwise referred to as gene therapy, are disclosed in N. Yang (“Gene Transfer into Mammalian Somatic Cells in vivo,” Crit Rev in Biotech, 12(4): 335-356 (1992)), the teachings of which are incorporated herein by reference. Gene therapy encompasses incorporation of DNA sequence(s) into somatic cells or germ line cells for use in either ex vivo or in vivo therapy. Gene therapy functions to replace genes, augment normal or abnormal gene function, and to combat I infectious diseases and other pathologies. Strategies for treating these medical problems with gene therapy include therapeutic strategies such as identifying the defective gene and then adding a functional gene to either replace the function of the defective gene or to augment a slightly functional gene; or prophylactic strategies, such as adding a gene for the product protein that will treat the condition or that will make the tissue or organ more responsive or susceptible to a treatment regimen. As an example of a prophylactic strategy, a gene such as that encoding one or more of the cell proliferation modulating proteins may be placed in a patient and thus prevent the occurrence of uncontrolled cell division or of metastasis or a gene that makes cells more sensitive to radiation could be inserted and then radiation of the tissue containing those cells would cause increased killing of the tumor cells, for example, epithelial cells of the prostate.

Many protocols for transfer of the DNA or regulatory sequence(s) of the cell proliferation modulating proteins are envisioned in this invention. Transfection of promoter sequences, other than one normally found specifically associated with the cell adhesion-mediating proteins or other sequences which increase the production of the cell adhesion-mediating protein(s) are envisioned as methods of gene therapy. An example of this technology is found in Transkaryotic Therapies, Inc., Cambridge, Mass. using homologous recombination to insert a “genetic switch” that turns on an erythropoietin gene in cells (see Genetic Engineering News, Apr. 15, 1994). Such “genetic switches” could be used to activate the expression of cell adhesion-mediating protein in cells not normally expressing those proteins or to increase expression of a cell adhesion-mediating protein.

Gene transfer methods for gene therapy fall into three broad categories: physical, (e.g., electroporation, direct gene transfer, and particle bombardment), chemical (e.g., lipid-bases carriers or other non-viral vectors) and biological (e.g., virus-derived vector and receptor uptake). For example, non-viral vectors may be used which include liposomes coated with DNA. Such liposome/DNA complexes are concentrated in the liver where they deliver the DNA to macrophages and Kupffer cells. These cells are long lived and thus provide long-term expression of the delivered DNA. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then re-implanted in the patient. In in vitro gene transfer, the transformed cells are cells growing in culture, such as tissue cell, and not particular cells obtained from a particular patient. These “laboratory cells” are transfected, the transfected cells are selected and expanded for either implantation into a patient or for other uses.

In vivo gene transfer involves introducing he DNA into the cells of the patient when the cells are within the patient. Methods include using virally mediated gene transfer using non-infectious virus to deliver the gene in the patient or injecting naked DNA into a site in the patient and the DNA is taken up by a percentage of cells in which the gene product protein is then expressed. Additionally, the other methods described herein such as use of a “gene gun,” may be used for in vitro insertion of the DNA or regulatory sequences controlling production of the cell adhesion-mediating protein(s).

Chemical methods of gene therapy may involve a lipid based compound, not necessarily a liposome, to transfer the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then the target cells that have the receptor will specifically bind the ligand and transport the ligand-DNA complex into the cell.

Many gene therapy methodologies employ viral vectors to insert gene sequences into cells. For example, altered retrovirus vectors have been used in ex vivo methods to introduce genes into peripheral and tumor-infiltrating lymphocytes, hepatocytes, epidermal cells, myocytes or other somatic cells. These altered cells are then introduced into the patient to provide the gene product from the inserted DNA.

Viral vectors have also been used to insert genes into cells using in vivo protocols. To direct the tissue-specific expression of foreign genes, cis-acting regulatory elements or promoters that are known to be tissue-specific can be used. Alternatively, this can be achieved using in situ delivery of DNA viral vectors to specific anatomical sites in vivo. For example, gene transfer to blood vessels in vivo has been demonstrated by implanting in vitro transduced endothelial cells in chosen sites on arterial walls. The virus infected surrounding cells also express the gene product. A viral vector can be delivered directly to the in vivo site, by catheter for example, thus allowing only certain areas to be infected by the virus, and providing long-term, site-specific expression. In vivo gene transfer using retrovirus vectors has also been demonstrated in mammary tissue and hepatic tissue by injection of the altered virus into blood vessels leading to organs.

Viral vectors that have been used for gene therapy protocols include but are not limited to, retroviruses, other RNA viruses such as poliovirus or Sindbis virus, adenovirus, adeno-associated virus, herpes viruses, SV40, vaccinia, and other DNA viruses. Replication-defective murine retroviral vectors are the most widely utilized gene transfer vectors. Murine leukemia retroviruses are composed of a single strand RNA complexed with a nuclear core protein and polymerase (pol) enzymes, encased by a protein core (gag) and surrounded by a glycoprotein envelope (env) that determines host range. The genomic structure of retroviruses include the gag, pol, and env genes enclosed by the 5′ and 3′ long terminal repeats (LTR). Retroviral vector systems exploit the fact that a minimal vector containing the 5′ and the 3′ LTRs and the packaging signal are sufficient to allow vector packaging, infection, and integration into target cells providing that the viral structural proteins are supplied in trans form in the packaging cell line. Fundamental advantages of retroviral vectors for gene transfer include efficient infection and gene expression in most cell types, precise single copy vector integration into target cell chromosome DNA and ease of manipulation of the retroviral genome.

The adenovirus is composed of linear, double stranded DNA complexed with core proteins and surrounded with capsid proteins. Advances in molecular virology have led to the ability to exploit the biology of these organisms to create vectors capable of transducing novel genetic sequences into target cells in vivo. Adenoviral-based vectors will express gene produce proteins at high levels. Adenoviral vectors have high efficiencies of infectivity, even with lower titers of virus. Additionally, the virus is fully infective as a cell-free virion so injection of producer cell lines is not necessary. Another potential advantage to the adenoviral vector is the ability to achieve long-term expression of heterologous genes in vivo.

Mechanical methods of DNA delivery include fusogenic lipid vesicles such as liposomes or other vesicles for membrane fusion, lipid particles of DNA incorporating cationic lipids such as lipofectin, polylysine-mediated transfer of DNA, direct injection of DNA, such as by microinjection of DNA into germ cells or somatic cells, pneumatically delivered DNA-coated particles such as gold particles used in a “gene gun,” and inorganic chemical approaches such as calcium phosphate transfection. Particle-mediated gene transfer methods were first used in transforming plant tissue. With a particle bombardment device or “gene gun,” a motive force is generated to accelerate DNA-coated high density particles (such as gold or tungsten) to a high velocity that allows penetration of the target organ, tissue or cell. Particle bombardment can be used with in vitro systems or with ex vivo or in vivo techniques to introduce DNA into cells, tissues, and organs. Another method, ligand-mediated gene therapy, involves complexing the DNA with specific ligands to form ligand-DNA conjugates, to direct the DNA to a specific cell or tissue.

It has been found that injecting plasmid DNA into muscle cells yields a high percentage of cells that are transfected and have sustained expression of marker genes. The DNA of the plasmid may or may not integrate into the genome of the cells. Non-integration of the transfected DNA would allow the transfection and expression of gene product proteins in terminally differentiated tissue for a prolonged period of time without fear of mutational insertions, deletions or alterations in the cellular or mitochondrial genome. Long-term, but not necessarily permanent, transfer of the therapeutic genes into specific cells may provide treatments for genetic diseases or for prophylactic use. The DNA could be re-injected periodically to maintain the gene product level without mutations occurring in the genomes of the recipient cells. Non-integration of exogenous DNA sequence may allow for the presence of several different exogenous DNA constructs within one cell with all of the constructs expressing various gene products.

Electroporation for gene transfer uses an electrical current to make cells or tissues susceptible to electroporation-mediated gene transfer. A brief electric impulse with a given field strength is used to increase the permeability of the cell membrane in such as way that DNA molecules can enter the cell. This technique can be used in in vitro systems or with ex vivo or in vivo techniques to introduce DNA into cells, tissues and organs.

Carrier-mediate gene transfer in vivo can be used to transfect foreign DNA into cells. The carrier-DNA complex can be conveniently introduced into body fluids or the bloodstream and then site-specifically directed to the target organ or tissue in the body. Both liposomes and polycations, such as polylysine, lipofectins or cytofectins, can be used. Liposomes can be developed which are cell specific or organ specific and thus the foreign DNA carried by the liposome that will be taken up by target cells. Injection of immunoliposomes that are targeted to a specific receptor on certain cells can be used as a convenient method of inserting DNA into cells bearing the receptor. Another carrier system that has been used is the asialoglycoprotein/polylysine conjugate system for carrying DNA to hepatocytes for in vivo gene transfer.

The transfected DNA may also be complexed with other kinds of carriers so that the DNA is carried to the recipient cell and then resides in the cytoplasm or nucleoplasm. DNA can be coupled to carrier nuclear proteins in specifically engineered vesicle complexes and carried directly to the nucleus.

Gene regulation of the cell adhesion-mediating proteins may be accomplished by administering compounds that bind to the gene encoding one of the cell adhesion-mediating proteins or to the control regions associated with the gene or to its corresponding RNA transcript to modify the rate of transcription or translation. Additionally, cells transfected with a DNA sequence encoding the cell adhesion-mediating protein(s) may be administered to a patient to provide an in vivo source of that protein(s). For example, cells may be transfected with a vector containing a nucleic acid sequence encoding the cell adhesion-mediating protein. The transfected cells may be cells derived from the patient's normal tissue, the patient's diseases tissue or may be non-patient cells.

For example, tumor cells removed from a patient can be transfected with a vector capable of expressing a protein(s) or a fragment of the present invention, and re-introduced into the patient. The transfected tumor cell would then produce levels of protein in the patient that inhibit the growth of the tumor. Patients may be human- or non-human animals. Cells may also be transfected by non-vector or physical or chemical methods known in the art such as electroporation, ionoporation or via a “gene gun.” Additionally, the DNA may be directly injected, without the aid of a carrier, into a patient. In particular, the DNA may be injected into skin, muscle or blood.

The gene therapy protocol for transfecting the cell adhesion-mediating proteins into a patient may be either through integration of the DNA encoding the cell adhesion-mediating protein of the present invention into the genome of the cells, into minichromosomes or as a separate replicating or non-replicating DNA construct in the cytoplasm or nucleoplasm of the cell. Expression of the cell adhesion-mediating protein may continue for a long period of time or re-injection of the DNA may be provided periodically to maintain the desired level of protein(s) in the cell, the tissue or organ or a determined blood level.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents. Alternatively, the inventive polypeptides and their fragments may be used as targets in a microarray.

Microarrays may be prepared, used, and analyzed using methods known in the art such as for example, as described in Brennan, T. M. et al., U.S. Pat. No.: 5,474,796; Schena, M. et al., Proc. Natl. Acad. Sci. USA, 93:10614-10619 (1996); Baldeschweiler et al., WO95/251116; Shalon, D. et al., WO95/35505; Heller, R. A. et al,. Proc. Natl. Acad. Sci. USA, 94:2150-2155 (1997); and Heller, M. J. et al., U.S. Pat. No.: 5,605,662, the disclosures of each of which is incorporated herein by references in its entirety.

In addition, the invention encompasses antibodies and antisera, that can be used for testing for the presence or absence of the cell adhesion-mediating proteins or amino acid sequences. Such antibodies and antisera can also be used in diagnosis, prognosis or treatment of diseases and conditions characterized by or associated with neoplastic activity or lack thereof. Such antisera and antibodies can also be used to decrease tumor growth and/or metastasis where desired, in tumor tissue, and to detect or localize tumor growth when tagged with a reporter molecule.

The polypeptides, their fragments or other derivatives or analogs thereof or cells expressing them can also be used as immunogens to produce antibodies thereto. These antibodies can be, for example, polyclonal or monoclonal antibodies. The present invention also includes chimeric, single chain, and humanized antibodies, as well as Fab fragments or the product of an Fab expression library. Various procedures known in the art may be used for the production of such antibodies and fragments.

Antibodies generated against the polypeptides corresponding to a sequence of the present invention can be obtained by direct injection of the polypeptides into an animal or by administering the polypeptides to an animal, preferably a nonhuman. The antibody so obtained will then bind the polypeptide itself. In this manner, even a sequence encoding only a fragment of the polypeptide can be used to generate antibodies binding the whole native polypeptide. Such antibodies can then be used to isolate the polypeptide from tissue expressing that polypeptide.

For preparation of monoclonal antibodies, any technique which provides antibodies produced by continuous cell line cultures can be used. Examples include the hybridoma technique (Kohler, et al., Nature, 256: 495-497 (1975)), the trioma technique, the human B-cell hybridoma technique (Kozbor, et al., Immunology Today, 4:72 (1983)) and the EBV-hybridoma technique to produce human monoclonal antibodies (Cole, et al., in Monoclonal Antibodies and Cancer Therapy, Alan R. Liss, Inc., pg. 77-96 (1985)).

Techniques described for the production of single chain antibodies such as for example, those described in U.S. Pat. No.: 4,946,778, the disclosure of which is incorporated by reference herein in its entirety, can be adapted to produce single chain antibodies to immunogenic polypeptide products of this invention. Also, transgenic mice or other organisms including other mammals, can be used to express humanized antibodies to immunogenic polypeptide products of this invention.

The above-described antibodies can be employed to isolate or to identify clones expressing the polypeptide or purify the polypeptide of the present invention by attachment of the antibody to a solid support for isolation and/or purification by affinity chromatography.

Such antibodies and antisera can be combined with pharmacologically acceptable compositions and carriers to form diagnostic, prognostic or therapeutic compositions. The term “antibody” or “antibody molecule” refers to a population of immunoglobulin molecules and/or immunologically active portions of immunoglobulin molecules, i.e., molecules that contain an antibody combining site or a paratope.

Passive antibody therapy using antibodies that specifically bind the cell adhesion-mediating proteins can be employed to modulate cancer-related processes. In addition, antisera directed to the Fab regions of antibodies of the cell adhesion-mediating proteins can be administered to block the ability of endogenous antisera to the proteins to the proteins.

Cell adhesion-mediating proteins of the present invention also can be used to generate antibodies that are specific for the inhibitor(s) and receptor(s). These antibodies can be either polyclonal antibodies or monoclonal antibodies. These antibodies that specifically bind to the cell adhesion-mediating proteins or their receptors can be used in diagnostic methods and kits that are well known to those of ordinary skill in the art to detect or to quantify the cell adhesion-mediating proteins or their receptors in a body fluid or tissue. Results from these tests can be used to diagnose or predict the occurrence or reoccurrence of a disease state such as for example, cancer or other uncontrolled cell division/growth mediated diseases.

The invention also includes use of the cell adhesion-mediating proteins, antibodies to those proteins, and compositions comprising those proteins and/or their antibodies in diagnosis or prognosis of diseases characterized by uncontrolled cell division. As used herein, the term “prognostic method” means a method that enables a prediction regarding the progression of a disease if a human or non-human animal diagnosed with the disease, in particular a cell proliferation-dependent disease. The term “diagnostic method” as used herein means a method that enables a determination of the presence or type of cell proliferation-dependent disease characterized by neoplastic growth in or on a human or non-human animal.

Cell adhesion-mediating proteins of the present invention can be synthesized on a standard microchemical facility and the purity of the synthetic proteins can be checked using HPLC and mass spectrophotography. Methods of protein synthesis, HPLC purification and mass spectrophotography are commonly known to those of ordinary skill in these arts. The cell adhesion-mediating proteins and their receptors may also be produced using recombinant E. coli or yeast expression systems, and purified with column chromatography.

Different protein fragments of the intact cell adhesion-mediating proteins can be synthesized for use in several applications including, but not limited to the following: as antigens for the development of specific antisera, as agonists and antagonists active at binding sites of the cell adhesion-mediating protein, as proteins linked to or used in combination with, cytotoxic agents for targeted killing of cells that bind the cell adhesion-mediating proteins.

The synthetic protein fragments of the cell adhesion-mediating proteins have a variety of uses. The protein that binds to the receptor(s) of the cell adhesion-mediating proteins with high specificity and avidity is radiolabeled and employed for visualization and quantitation of binding sites using autoradiographic and membrane binding techniques. This application provides important diagnostic and research tools. Knowledge of the binding properties of the receptor(s) facilitates investigation of the transduction mechanisms linked to the receptor(s).

The cell adhesion-mediating proteins and proteins derived from them can be coupled to other molecules using standard methods. The amino and carboxyl termini of the cell proliferation modulating proteins both contain tyrosine and lysine residues and may be isotopically and nonisotopically labeled using many techniques, for example, radiolabeling using conventional techniques (tyrosine-residues—chloroamine T, iodogen, lactoperoxidase; lysine-residues—Bolton-Hunter reagent). These coupling techniques are well known to those of skill in the art. Alternatively, tyrosine or lysine is added to fragments that do not have these residues to facilitate labeling of reactive amino and hydroxyl groups on the protein. The coupling technique is chosen on the basis of the functional group available on the amino acids including but not limited to, amino, sulfhydral, carboxyl, amide, phenol, and imidazole. Various reagents used to effect these couplings include among others, glutaraldehyde, diazotized benzidine, carboiimide, and p-benzoquininone.

The cell adhesion-mediating proteins are chemically coupled to isotopes, enzymes, carrier proteins, cytotoxic agents, fluorescent molecules, chemiluminescent molecules, bioluminescent molecules and other compounds for a variety of applications. The efficiency of the coupling reaction is determined using different techniques appropriate for the specific reaction. For example, radiolabeling of a protein of the present invention with 125I is accomplished using chloroamine T and Na 125I of high specific activity. The reaction is terminated with sodium metabisulfite and the mixture is desalted on disposable columns. The labeled protein is eluted from the column and fractions are collected. Aliquots are removed from each fraction and radioactivity is measured in a gamma counter. In this manner, the unreacted Na125I is separated from the labeled protein. The protein fractions with the highest specific activity are stored for subsequent use such as for example, in analysis of the ability to bind to antisera of the cell proliferation modulating proteins.

In addition, labeling the cell adhesion-mediating proteins with short-lived isotopes enables visualization of binding sites in vivo using positron emission tomography or other modem radiographic techniques to locate tissues with binding sites for the cell adhesion-mediating protein(s).

Systematic substitution of amino acids within these synthesized proteins yields high affinity protein agonists and antagonists of the cell proliferation -modulating proteins that enhance or diminish binding.

Clones 128375 and PCEA2, which can be used to obtain polynucleotide sequences SEQ ID NO: 1 and 54, respectively, were deposited with the American Type Culture Collection (10801 University Boulevard, Manassas, Va. 20110-2209, USA) as an original deposit under the Budapest Treaty. Clone 128375 depostited on Jun. 13, 2001 was given accession number PTA-3456. Clone PCEA2 deposited on Jul. 30, 2001 was given accession number PTA-3572. The deposit(s) referred to herein will be maintained under the Budapest Treaty on the International Recognition of the Deposit of Microorganisms for the purposes of Pat Procedure for the required time and will become available in accordance with that Treaty.

Regarding PCEA2 cDNA, more than one polynucleotide sequence is included in the ATCC deposit of lyophilized cDNA. The PCEA2 cDNA may be separated by size (molecular weight) on a polyacrylamide gel by known methods (“Molecular Cloning: A Laboratory Manual,” Sambrook J, Fritsch E F, and Maniatis T, Cold Spring Harbor Laboratory Press (1989)). For example, digest the DNA with the restriction enzymes EcoRI and NotI then run the product on a 1% agarose gel with a 1 kb ladder as a size marker (for example, Catalog No: N3232S, New England Biolabs, Beverly, Mass.); the PCEA2 insert is 2.2 kb in size, the plasmid vector is 4.2 kb in size and the unrelated plasmid inserts are 0.5 and 1.8 kb in size.

### EXAMPLES

### Example 1

### Construction of cDNA Libraries Total RNA Creation

Human prostate tissue was used as a source for RNA. Tissue was homogenized in either TRIZOL (Cat. No: 15596-018 GIBCO-BRL, Bethesda, Md.) reagent or TRI-REAGENT (Cat. No: TRI 18 Molecular Research Center, Cincinnati, Ohio); both are solutions of phenol and guanidine isothiocyanate, at a concentration of 2 g tissue/20 mL reagent with a Polytron probe (Brinkmann Instruments, Westbury, N.Y.). The homogenate was incubated briefly at room temperature. 4 mL of chloroform were added and again incubated briefly at room temperature prior to centrifugation. The aqueous phase was transferred to a new tube and precipitated with isopropyl alcohol. The RNA was then resuspended in 0.5% SDS.

mRNA was prepared from the total RNA using the polyA Pure kit from (Cat. No: 1915, Ambion Austin, Tex.) according to manufacturer's instructions.

cDNA library creation.

cDNA was created from the mRNA extracted as described above. Library creation was accomplished with a proprietary protocol including as described in U.S. Pat. Nos.: 5,162,209 and 5,643,766, the teachings of which are incorporated herein by reference.

### Example 2

### Isolation and Sequencing of cDNA

Libraries were plated on Luria Broth agar plates prepared by dissolving 20 g of Bacto Luria Broth, Lennox (Cat. No: 0402-08-0, Becton Dickinson and Company, Franklin Lakes, N.J.), and 15 g/l BactQ-Agar (Cat. No: 0140-01, Becton Dickinson and Company, Franklin Lakes, N.J.) in distilled water containing 100 g/ml carbenicillin (Cat. No: C-1389, Sigma, St. Louis, Mo.). Colonies were grown for 20 hours at 37° C. All colonies on the plates were picked using a Biopick robot BP600 (Biorobotics Ltd, Cambridge, UK) into Terrific Broth (in “Molecular Cloning: A Laboratory Manual,” 1989, Sambrook J, Fritsch E F, and Maniatis T, Cold Spring Harbor Laboratory Press) containing 100 g/ml ampicillin and grown at 37° C. for 40 h. DNA was prepared from the plasmids using an ATGC Alkaline Lysis Miniprep kit (Edge Biosystems, Gaithersburg, Md.) according to manufacturer's instructions. DNA sequencing reactions were run using a DNA Sequencing Kit (Cat. No: 4303154, PE Biosystems, Foster City, Calif.) in a Peltier Thermocycler Model PTC-225 (MJ Research, Watertown, Mass.). Reaction products were purified on a G50 column and resuspended in loading buffer consisting of 10 ml formamide and 2 ml of Blue Dextran disodium ethylenediaminetetra-acetate. The mixture was loaded onto an acrylamide gel prepared according to manufacturer's instructions and run on an ABI 377 Sequencer (Applied Biosystems, Foster City, Calif).

### Example 3

### Homology Searching of DNA and Deduced Proteins

SEQ ID NOs: 1-73 were compared to known sequences using the Basic Local Alignment Search Tool BLAST (Altschul, S F, J Mol Evol, 36:290-300 (1993); and Altschul et al,. J Mol Biol, 215:403-410, (1990)), “BLASTN” compares nucleotide sequences. These were done against the nucleotide sequence databases GenBank, GenBank ESTs, and GenEmbl on an internal Alphagene server. These databases contain previously identified and annotated sequences and were updated weekly. “BLASTP” compares amino acid sequences. These were done against protein sequence databases Swissprot, PIR, Patchx, and Genpept on an internal Alphagene server. These databases contain previously identified and annotated sequences and were updated whenever a new release became available. BLAST evaluated the statistical significance of any matches found, and reported only those matches that satisfied the user-selected threshold of significance. In this application, the threshold was set at 10 for nucleotides and for amino acids.

When a homologous sequence was found, comparisons between the two sequences were made using the GAP program from the Wisconsin Package Version 10.1, Genetics Computer Group, Madison, Wis. GAP uses the alogorithm of Needleman and Wunsch (J Mol Biol, 48:443-453 (1970)) to find the alignment of two entire sequences that maximizes matches and minimizes gaps. The default values of 50 for the gap creation penalty and 3 for the gap extension penalty were used.

Another approach used for detecting protein homologies was to compare an amino acid sequence to a database of protein motifs created from groups of related molecules by using Hidden Markov Models (HMM) (Krogh, A et al., J Mol Biol, 235:1501-1531 (1994); Eddy, SR Bioinformatics, 14:755-763, (1998)). The Pfam database (Bateman A, et al., Nucleic Acids Research, 28:263-266 (2000)) was searched on an internal Alphagene server and was updated whenever a new version was released. A nucleic acid sequence can be translated in all possible frames on both strands and the resulting translated amino acid sequence used to search the Pfam database. Two kinds of motifs are found in the Pfam database: PfamA's which are assigned permanent accession identifiers and are annotated, and PfamB's that are entirely computer generated from the sequence databases, are not annotated, and are assigned only temporary identifiers that change with each release. The significance of PfamB matches were determined by accessing the Pfam website at the Sanger Center on the world wide web at sanger.ac.uk/cgi-bin/Pfam and examining the molecules used to generate the motif, and the location(s) of the motif within those molecules.

### Example 4

### Peptide Sequence Analysis

Analysis of peptide sequences, including SEQ ID NOs: 2, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71 and 73 was accomplished using the PeptideStructure. PeptideSort and PlotStructure programs from the Wisconsin Package Version 10.1, Genetics Computer Group, Madison, Wis. PeptideStructure calculated hydrophilicity after the option to use the algorithm of Kyte and Doolittle (J Mol Biol, 157:105-32 1982) was selected. The default setting for the window of seven residues was used. PeptideStructure calculated antigenicity using the methods of Jameson and Wolf (CABIOS 4:181-6 (1988)). PeptideStructure predicted glycosylation sites where the residues have the composition NXT or NXS. When X is D, W or P the sites were noted as a weak glycosylation sites, all other combinations were considered strong. Plotstructure displayed the results of the PeptideStructure program in graphic form. PeptideSort uses the entire amino acid composition of the polypeptide to calculates an exact molecular weight and isoelectric point.

### Example 5

### Gene Prediction

The GENSCAN program was used to predict genes from genomic DNA. GENSCAN was developed by Chris Burge in 1997 in the research group of Samuel Karlin, Department of Mathematics, Stanford University (Bioinformatics 15(11):887-899 (1999)). The program is widely used for predicting genes and proteins from genomic sequences. The software has been tested on human genomic sequence in-house and was chosen for giving the best performance. The input sequence was stated to be human in origin. The nucleotide sequence was displayed as well as polypeptide. Exons were then found by using intron/exon boundaries and other splicing motifs to find the polynucleotide used to deduce the polypeptide. The exons were assembled into a polypeptide.

### Example 6

### Chromosomal Localization

The cDNA from clones 128375 was matched to genomic BAC sequences by BLASTN. These BACs were localized on the human genome using the information available from NCBI on the world wide web at ncbi.nlm.nih.gov following the human genome resources link.

### Example 7

### Northern Analyses

Northern analyses were performed using two blots containing human RNA from multiple tissues. These blots were purchased from Clontech (Cat. Nos: 7780-1 and 7784-1, Palo Alto, Calif.) and contained lug of human poly A+ mRNA per lane with size markers indicated on the blots. Probe was made by random priming using a High Prime DNA labeling kit (Cat. No: 1585584, Roche Diagnostics, Indianapolis, IN) according to manufacturers instructions utilizing the full DNA sequence given in SEQ ID NO: 1. Hybridization was overnight at 45° C. according to manufacturer's instructions in Ambion Ultrahyb (Cat. No: 8670). The blot was washed at 50° C. for 1 hour in 0.1×SSC (in “Molecular Cloning: A Laboratory Manual,” 1989, Sambrook J, Fritsch E F and Maniatis T, Cold Spring Harbor Laboratory Press) and 0.1% sodium dodecyl sulfate.

### Example 8

### Clone 128375

A sequence of the present invention is SEQ ID NO: 1 provided from clone “128375.” Clone 128375 was identified from a cDNA library created from human prostate tissue as described above. The cDNA insert of clone 128375 was deposited under ATCC accession number PTA-3456 on Jun. 13, 2001.

The XhoI/NotI restriction fragment for clone 128375 is about 1435 base pairs. The nucleotide sequence of this insert is represented as SEQ ID NO: 1. A complete open reading frame is present with a starting methionine and a stop codon. This open reading frame begins at nucleotide 115 and ends at nucleotide 1329 with a stop codon from nucleotides 1330 through 1332. This sequence encodes a polypeptide 405 amino acids in length. The deduced amino acid sequence encoded by this nucleotide sequence is shown in SEQ ID NO: 2. A second open reading frame is present that lacks a starting methionine. This second open reading frame begins at nucleotide 1 and ends at nucleotide 1329 with a stop codon at from nucleotides 1330 through 1332. This sequence encodes a polypeptide that is 443 amino acids in length. The deduced amino acid sequence encoded by this nucleotide sequence is shown in SEQ ID NO: 3. This amino acid sequence is identical to the amino acid sequence of SEQ ID NO: 2 except that it contains an additional 38 amino acids at the amino terminus.

A “BLASTN” analysis of SEQ ID NO: 1 indicated no significant homology to any expressed human nucleotide sequences, only to human genomic DNA sequence. Genomic BAC clones AC073898 and AC069278 have regions of exact matches to SEQ ID NO: 1. These BAC clones are stated to be from chromosome 19. GenBank accession number AK018613 stated to be murine adult cecum cDNA also showed some homology to SEQ ID NO: 1.

A “BLASTP” analysis of SEQ ID NO: 2 showed greatest sequence homology to a protein sequence having GenBank accession number BAB31307, that was also referred to as murine adult male cecum cDNA whose nucleotide sequence is given in GenBank accession number AK018613. A GAP alignment of BAB31307 with SEQ ID NO: 2 revealed that SEQ ID NO: 2 from amino acids 1 to 405 aligned with BAB31307 from amino acids 162 to 577 with 56% identity and 59% similarity.

The “BLASTP” analysis further revealed that SEQ ID NO: 2 showed sequence homology to many proteins of the CEA family including GenBank accession number Q15600 stated to be human TM2-CEA Precursor protein, GenBank accession number AAC18434 stated to be human BGP1, GenBank accession number AAA52607 stated to be human pregnancy-specific beta-1 glycoprotein, GenBank accession number AAB59513 stated to be carcinoembryonic antigen precursor from Homo sapiens and GenBank accession number P40199 stated to be human normal cross-reacting antigen precursor. Since similarity in protein sequence frequently implies shared function SEQ ID NO: 2 is presumed to share at least some functional similarity with these similar sequences. This family is well characterized for its utility as markers for cancer (Hammerstrom, Seminars in Cancer Biology, 9:67-81 (1999); Nakopoulou et al., Dis Colon Rectum, 26:269-74 (1983); Lechner et al., J Am Coll Surg, 191:511-8 (2000); Yamao et al., Jpn J Clin Oncol, 29:550-5, (1999)) and for its function in cell adhesion, signaling and angiogenesis (reviews, Obrink, Current Opinion in Cell Biology, 9:616-626 (1997); Wagener and Ergun, Experimental Cell Research, 261:19-24 (1990)).

A Gap alignment of Q15600 with SEQ ID NO: 2 revealed that SEQ ID NO: 2 from amino acid 1 to amino acid 369 aligned with Q15600 from amino acids 73 to 430 with 32% identity and 40% similarity. A Gap alignment of AAC 18434 with SEQ ID NO: 2 revealed amino acids 1 to 365 of SEQ ID NO: 2 aligned with AAC18434 from amino acids 73 to 428 with 32% identity and 40% similarity. A Gap alignment of AAA52607 with SEQ ID NO: 2 revealed that SEQ ID NO: 2 from amino acid 1 to 271 aligns with AAA52607 from amino acids 162 to 428 with 34% identity and 42% similarity. A Gap alignment of AAB59513 with SEQ ID NO: 2 revealed that SEQ ID NO: 2 from amino acids 1 to 273 aligned with AAB59513 from amino acids 428 to 702 with 33% identity and 40% similarity. A Gap alignment of p40199 with SEQ ID NO: 2 revealed that SEQ ID NO: 2 from amino acids 1 to 283 aligned with p40199 from amino acids 73 to 344 with 33% identity and 41% similarity.

The BLASTP analysis also revealed that SEQ ID NO: 2 showed sequence homology to the following proteins: CAA32940 stated to be TM2-CEA precursor [Homo sapiens]; CAA02706 stated to be unnamed protein product [Homo sapiens]; AAA62835 stated to be carcinoembryonic antigen [Homo sapiens]; AAA51963 stated to be carcinoembryonic antigen precursor [Homo sapiens]; CAA34474 stated to be pCEA80-11 protein (647 AA) [Homo sapiens]; PSG4_Human stated to be pregnancy-specific beta-1-glycoprotein 4 precursor (PSBG-4) (PSBG-9); PSG3_Human stated to be pregnancy-specific beta-1-glycoprotein 3 precursor (PSBG-3) (carcinoembryonic antigen SG5); AAC60584 stated to be pregnancy-specific beta 1-glycoprotein, PSG {clone hIS25} [human, colon, Peptide, 428 aa] [Homo sapiens]; CAA01646 stated to be trophoblast membrane expressed protein [Homo sapiens]; AAA52606 stated to be pregnancy-specific beta-1 glycoprotein precursor [Homo sapiens]; P06731 stated to be carcinoembryonic antigen precursor (CEA) (meconium antigen 100) (CD66E antigen); AAC60584 stated to be pregnancy-specific beta 1-glycoprotein, PSG {clone hIS25} [human, colon, Peptide, 428 aa] [Homo sapiens]; AAA52606 stated to be pregnancy-specific beta-1 glycoprotein precursor [Homo sapiens]; B33258 stated to be pregnancy-specific glycoprotein 1 precursor variant d-human; E43354 stated to be pregnancy-specific glycoprotein I form c—human (fragment); A35341 stated to be pregnancy-specific beta-1 glycoprotein 1d precursor-human; A35964 stated to be pregnancy-specific glycoprotein 1 form d precursor-human; A34595 stated to be pregnancy-specific beta-1-glycoprotein 12 precursor, placental—human; AAA66960 stated to be carcinoembryonic antigen SG9 [Homo sapiens]; AAA60204 stated to be fetal liver non-specific cross-reactive antigen precursor protein [Homo sapiens]; AAA60203 stated to be PSG11 [Homo sapiens]; AAC25488 stated to be PBGC_HUMAN [Homo sapiens]; AAA59915 stated to be normal cross-reacting antigen [Homo sapiens]; JC4122 stated to be pregnancy-specific glycoprotein 13′ precursor—human; AAA60195 stated to be pregnancy-specific beta-1 glycoprotein [Homo sapiens]; CAA35612 stated to be pregnancy-specific beta-1 glycoprotein (AA 1-426) [Homo sapiens]; B54312 stated to be pregnancy-specific beta-1 glycoprotein 4 precursor, placental (clone hPS133)−human; AAA75299 stated to be pregnancy-specific glycoprotein 13 [Homo sapiens]; S09016 stated to be pregnancy-specific glycoprotein beta-1 precursor—human; C5 5181 stated to be pregnancy-specific beta-1-glycoprotein 11 form s precursor—human; D43 354 stated to be pregnancy-specific glycoprotein I form b—human (fragment); C43354 stated to be pregnancy-specific glycoprotein I form a—human (fragment); AAHO5008 stated to be carcinoembryonic antigen-related cell adhesion molecule 6 (non-specific cross reacting antigen) [Homo sapiens]; AAA51739 stated to be nonspecific cross-reacting antigen precursor [Homo sapiens]; AAA52605 stated to be pregnancy-specific beta-1 glycoprotein precursor [Homo sapiens]; B35334 stated to be pregnancy-specific beta-1-glycoprotein 7 precursor—human; AAA59907 stated to be non-specific cross reacting antigen [Homo sapiens]; A33258 stated to be pregnancy-specific glycoprotein 1 precursor variant a—human; AAA36513 stated to be fetal liver non-specific cross-reactive antigen-2 precursor protein [Homo sapiens]; AAA36515 stated to be pregnancy-specific glycoprote in-1a [Homo sapiens]; AAC25489 stated to be PBGD_HUMAN; fetal liver non-specific cross-reactive antigen-2; FL-NCA-2 [Homo sapiens]; AAC25490 stated to be PBGi_HUMAN [Homo sapiens]; B36109 stated to be pregnancy-specific beta-1 glycprotein 10 precursor—human; D33258 stated to be pregnancy-specific beta-1 glycoprotein 6 precursor—human. Since similarity in protein sequence frequently implies shared function SEQ ID NO: 2 is presumed to share at least some functional similarity with these similar sequences.

A search using HMM and the Pfam database as described above revealed several protein motifs in SEQ ID NO: 2. The immunoglobulin domain model PF00047 was found to occur 3 times within SEQ ID NO: 2 with an overall matching score of 71.68. The first occurrence of the immunoglobulin domain within SEQ ID NO: 2 is from amino acids 3 through 53 similar to the PF00047 model from amino acids 1 through 45; the second match is from SEQ ID NO: 2 amino acids 92 to 147 to the PF00047 model from amino acids 3 through 45; the last is from SEQ ID NO: 2 from amino acids 189 to 239 to the PF00047 model from amino acids 1 through 45. Immunoglobulin domains are implicated in protein-protein and protein-ligand interactions. CEA molecules have variable numbers of immunoglobulin domains in their extracellular regions and are members of the immunoglobulin superfamily (review in Hammerstrom, ibid).

An unannotated Pfamb motif was found to match SEQ ID NO: 2 with a score of 39.46. SEQ ID NO: 2 from amino acids 88 to 165 aligned with amino acids 1 to 78 of the Pfamb motif. An investigation of the molecules used to generate this motif as described above revealed that it was generated from pregnancy specific glycoprotein beta-1 and its variants, members of the CEA family. The motif was interspersed with the immunoglobulin domains, comparable to its location within SEQ ID NO: 2.

A second unannotated Pfamb was found to match SEQ ID NO: 2 with a score of 34.09. SEQ ID NO: 2 from amino acids 263 to 297 aligned with amino acids 1 through 35 of the Pfamb motif. An investigation of the molecules used to generate this motif as described above revealed that it was generated from 18 sequences all from CEA family members including GenBank accession numbers: P13688 stated to be human biliary glycoprotein 1 precursor, Q15600 stated to be TM2-CEA precursor, P31809 stated to be murine biliary glycoprotein 1 precursor, Q03715 stated to be nonspecific cross-reacting antigen, P16573 stated to be rat ecto-ATPase precursor, and P40198 stated to be human carcinoembryonic antigen CGM1 precursor. The motif flanks and spans the transmembrane domain in all of these molecules, comparable to its location within SEQ ID NO: 2.

The PeptideStructure program, used as described above, showed a hydrophobic region in SEQ ID NO: 2 centered around amino acid 277 of sufficient size and hydrophobicity that is likely to function as a transmembrane spanning region. As noted above this region demonstrates a shared motif, including the transmembrane region and its flanking sequence, with members of the CEA family. The sequences N-terminal to these amino acids containing the immunoglobulin domains described above likely constitute an extracellular portion of the molecule. The amino acids C-terminal to this predicted transmembrane domain are likely to form a cytoplasmic domain.

The PeptideStructure program, as described above, also identified a number of potential sites for N-linked glycosylation within the predicted extracellular portion of SEQ ID NO: 2 with strong sites at asparagine residues at amino acids 101, 127, 189, and 236 and a weak site at 148. Members of the CEA family are known to be glycosylated (Paxton et al., PNAS, 84:920-924, (1987)).

The PeptideSort program, as described above, showed that SEQ ID NO: 2 had a molecular weight of 44,819.24 Daltons and an isoelectric point of 6.33. The cytoplasmic domains of CEA family members human biliary glycoprotein (CEACAM1) and mouse homologs C-CAM1 and C-CAM2 contain binding sites for calmodulin. All three of these molecules share a calmodulin-binding site in the cytoplasmic domain adjacent to the transmembrane domain (Edlund et al., J Biol Chem, 271:1393-1399). SEQ ID NO: 2 shared some sequence conservation in this region from amino acid 290 through 302 ‘FLYIRNARRPSRKT’ (SEQ ID NO: 74) including two charged amino acids at 300 and 301. Both murine homologs contain a second calmodulin-binding site closer to the C-terminus of the cytoplasmic tail that was not found in human biliary glycoprotein. A minimal calmodulin-binding motif ‘Hydrophobic-Q-X3-R’ (Aitken, Molecular Biotechnology, 12:241-53 (1999)) was found in a comparable location in SEQ ID NO: 2 from amino acids 348 to 353 ‘LQGRIR’ (SEQ ID NO: 75). Human biliary glycoprotein forms homodimers and this process is regulated by calmodulin (Edlund et al., J Biol Chem, 271:1393-1399). The presence of calmodulin binding sites or motifs may be inferred from sequence similarity and binding motifs found in SEQ ID NO: 2.

The serine found at amino acid 360 of SEQ ID NO: 1 matched the consensus sequence for phosphorylation targets of proline-directed cell-cycle kinases ‘S/T-P-X-K/R’; (Aitken, 1999, ibid) having ‘SPWK’ (SEQ ID NO: 76), from amino acids 360 through 363. SEQ ID NO: 1 also had three matches to the consensus motif ‘Y-X-X-hydrophobic’ to which SH2 domains can bind when the tyrosine is phosphorylated (Aitken, 1999, ibid). These motifs were found from amino acids 332 through 335 ‘YCNI’ (SEQ ID NO: 77), from amino acids 387 through 390 ‘YEEL’ (SEQ ID NO: 78), and from amino acids 398 through 401 ‘YIQI’ (SEQ ID NO: 79). Interactions between proteins via SH2 domains play a key role in signal transduction events and the SH2 binding sites provide targets for pharmacological intervention (Beattie, Cell Signal, 8:75-86 (1996)). Multiple SH2 domains contribute to binding specificity (Cowburn, Chem Biol, 3:79-82 (1996)).

Except for an additional 38 amino acids at the amino terminus, SEQ ID NO: 3 was found to have the same amino acid sequences as found in SEQ ID NO: 2. It therefore shares homologies to the same molecules, and contains the same HMM motifs with the same scores as SEQ ID NO: 2. Due to the extension on the amino terminus, it has slightly greater similarity to BAB31307 than SEQ ID NO: 2. A GAP alignment of SEQ ID NO: 3 with BAB31307 revealed that SEQ ID NO: 3 from 1 to 443 aligned with BAB31307 from amino acids 124 to 577 with 56% identity and 59% similarity.

A Gap alignment of Q15600 with SEQ ID NO: 3 revealed that SEQ ID NO: 3 from amino acid 1 to amino acid 430 aligned with Q15600 from amino acids 50 to 430 with 32% identity and 41% similarity. A Gap alignment of AAC18434 with SEQ ID NO: 3 from amino acids 1 to 298 aligned with AAC18434 from amino acids 219 to 526 with 30% identity and 38% similarity. A Gap alignment of AAA52607 with SEQ ID NO: 3 revealed that SEQ ID NO: 3 from amino acid 1 to 309 aligns with AAA52607 from amino acids 130 to 428 with 34% identity and 40% similarity. A Gap alignment of AAB59513 with SEQ ID NO: 3 revealed that SEQ ID NO: 3 from amino acids 1 to 321 aligned with AAB59513 from amino acids 397 to 702 with 34% identity and 40% similarity. A Gap alignment of p40199 with SEQ ID NO: 3 revealed that SEQ ID NO: 3 from amino acids 1 to 321 aligned with p40199 from amino acids 33 to 344 with 32% identity and 40% similarity.

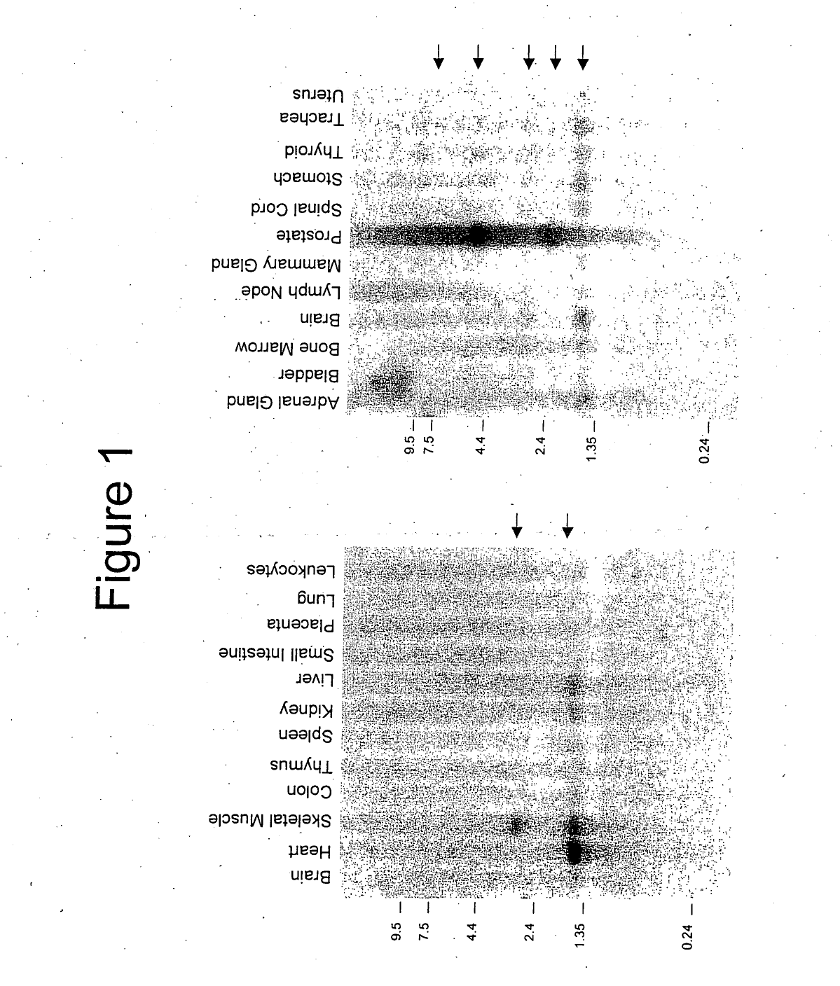

SEQ ID NO: 3 was shown by the PeptideSort program as described above to have a molecular weight of 48,873.76 Daltons and an isoelectric point of 5.65. Northern analyses were performed as described above and the results are shown in FIG. 1. Transcripts of several sizes are evident in the blot. An approximately 1.4 kilobase transcript was widely expressed in most of the tissues in the blot. In addition, skeletal muscle contained a transcript of about 3 kilobases. Prostate tissue showed two transcripts of unique sizes (approximately 4.6 and 2.0 kilobases) that were not evident in other tissues, as well as the 1.4 kilobase transcript. The expression of the 4.6 kilobase transcript was particularly strong.

SEQ ID NO: 1 mapped to chromosome 19 region 19q13.2. CEA family members have been mapped to chromosome 19 regions 19q13.1 and 19q13.2 flanking this area (Olsen et al., Genomics, 23:659-668 (1994); Thompson et al., Genomics, 12:761-772 (1992); Tynan et al., Nucleic Acids Research, 20:1629-1636; Teglund et al., Genomics, 23:669-684 (1994)) by methods that rely on cross-hybridization of known CEA genes with cosmids, and their assembly into contigs or by PCR. More distantly related family members with amino acid percent identity of 30-35% were not found by prior methods that relied on highly conserved nucleotide sequence.

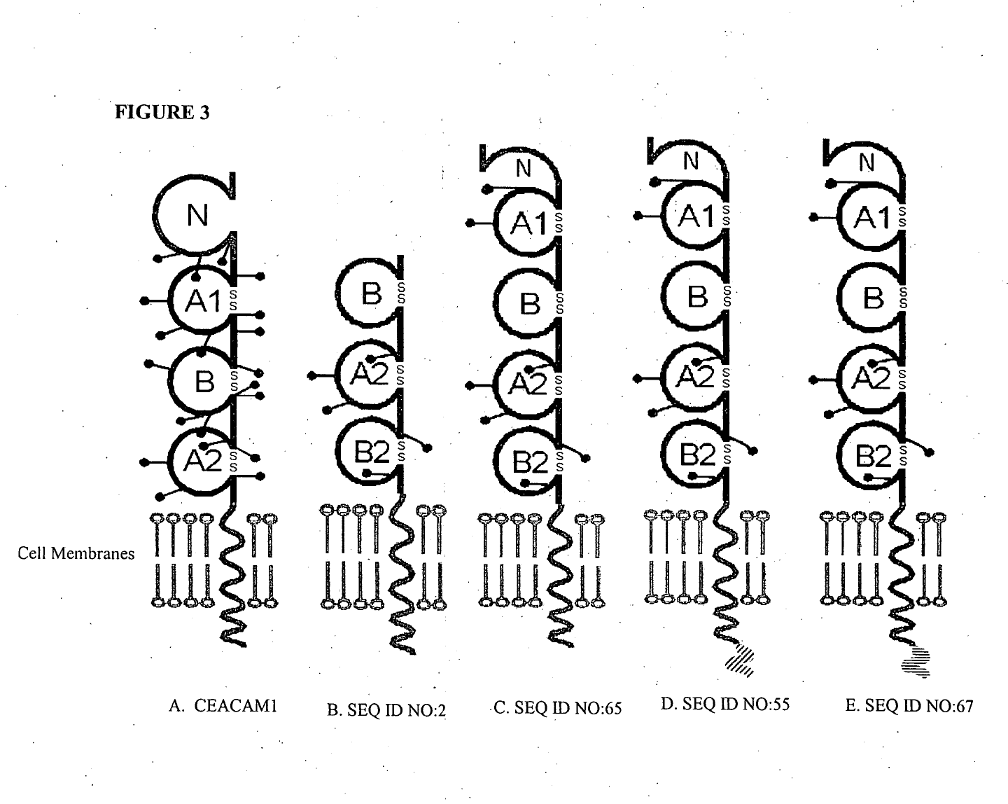

CEA family members exhibit a characteristic pattern of immunoglobulin domain distribution. SEQ ID NOs: 2 and 3 have three C-type immunoglobulin domains, of alternating B and A subtypes. A comparison of the domain structure of SEQ ID NOs: 2 and 3 with a known CEA family member CEACAM1 is given in FIG. 3.

Based upon the similarity of protein sequence to other CEA molecules, shared HMM motifs, similar patterns of Ig domain distribution, localization to chromosome 19 and calmodulin binding motifs, SEQ ID NO: 1 encodes a novel member of the CEA family. The encoded polypeptides SEQ ID NO: 2 and 3 are novel members of the CEA family. Other members of the CEA family are known to have altered levels of expression in numerous cancers (review, Hammerstrom ibid). Thus, SEQ ID NO: 1 and its expressed polypeptides SEQ ID NO: 2 and 3 are useful as tumor markers. Even absent differential expression in tumors, a polypeptide is useful as a tumor marker when it shows tissue specificity. Some CEA family members have been proven useful for immunolocalization of tumor tissue (, Nakopoulou et al., Dis Colon Rectum, 26:269-74 (1983)), in particular for radioimmunosurgery (Bertoglio et al., Seminars in Surgical Oncology, and for immunotherapy (Khare et al., Cancer Research, 61:370-5 (2001), Buchegger et al., Int J Cancer, 41:127-134 (1988)).

While SEQ ID NOs: 1, 2 and 3 share sequence similarities to other CEA family members, no cross-reactivity to known family members is expected at high stringency nucleic acid hybridization conditions due to the extent of unique sequence. Furthermore, specific antibodies that do not cross-react with known family members can be raised based upon the pattern of antigenic sites present in the polypeptides encoded by the polynucleotide in cDNA SEQ ID NO: 1 from prostate tissue. Since SEQ ID NO: 1 was isolated from human prostate tissue, shows strong expression in that tissue, and has tissue specific variants expressed in prostate tissue, SEQ ID NO: 1 and the polypeptides it encodes, SEQ ID NOs: 2 and 3 are useful as biomarkers of prostate tissue and can be used as markers for metastasized prostate tissue.

### Example 9

### Predicted Form of Novel CEA Family Member

A gene prediction process was utilized as described above. The BACs to which SEQ ID NO: 1 was localized, BAC AC073898 and BAC AC069278, overlapped by 279 nucleotides and were assembled into a contig. Genscan was run with this contig as input. The resulting predicted nucleotide sequence for the gene is provided as SEQ ID NO: 4.

SEQ ID NO: 4 contains a large open reading frame from nucleotides 1 to 3099 with a starting methionine at nucleotides 1 through 3 and a stop codon at 3100 through 3102. The peptide encoded by this open reading frame is given in SEQ ID NO: 5. A Gap alignment of SEQ ID NO: 5 and 2 revealed that SEQ ID NO: 5 was longer than SEQ ID NO: 2 on the amino terminus having 716 additional amino acids not found in SEQ ID NO: 2. Amino acid positions 717 to 973 of SEQ ID NO: 5 aligned with SEQ ID NO: 2 amino acid positions 1 to 257 with 100% identity. SEQ ID NO: 2 has an insertion comprising amino acids 258 to 266 between amino acids 973 and 974 of SEQ ID NO: 5. SEQ ID NO: 5 from amino acids 974 to 1005 matched SEQ ID NO: 2 from amino acids 267 to 298 with 100% identity. SEQ ID NO: 5 contains an additional 29 amino acids from 1006 to 1033 with little identity to SEQ ID NO: 2 from 298 to 405.

A Gap alignment of SEQ ID NO: 5 and 3 revealed that SEQ ID NO: 5 has 678 additional amino acids at the amino terminus not found in SEQ ID NO: 3. SEQ ID NO: 5 aligns from amino acid 679 to 973 to SEQ ID NO: 3 from amino acids 1 to 295 with 100% identity. SEQ ID NO: 3 was found to have an insertion of amino acids 296 to 304 between amino acids 973 and 974 of SEQ ID NO: 5. SEQ ID NO: 5 from amino acids 974 to 1005 matches to SEQ ID NO: 3 from amino acids 305 to 335 with 100% identity. SEQ ID NO: 5 contains an additional 29 amino acids from 1006 to 1033 with little identity to SEQ ID NO: 3 from 336 to 443.

A “BLASTN” analysis of SEQ ID NO: 4 revealed a match to GenBank accession number R94543, an EST stated to be cDNA from Soares fetal liver spleen of Homo sapiens. R94543 aligns with SEQ ID NO: 5 from nucleotides 1 to 214 of R94543 with nucleotides 1068 to 1281 of SEQ ID NO: 4 with 100% identity in this region. BLASTN also confirms that SEQ ID NO: 4 matches BAC AC073898 and BAC AC069278, the sequences from which it was generated.

A “BLASTP” analysis of SEQ ID NO: 5 showed greatest sequence homology to a protein sequence having GenBank accession number BAB31307 (described in Example 1). A Gap alignment of BAB31307 with SEQ ID NO: 5 revealed that SEQ ID NO: 5 from amino acids 538 to 1031 aligned with BAB31307 from amino acids 1 to 480 with 58% identity and 61% similarity.

The “BLASTP” analysis further revealed that SEQ ID NO: 5 showed sequence homology to many proteins of the CEA family including GenBank accession numbers: AAB59513 and AAC18434 stated to be human BGP1; CAA34404 stated to be human TM1-CEA preprotein; Swissprot accession number Q00888 stated to be human pregnancy-specific beta-1 glycoprotein 4 precursor, and accession number P40199 stated to be human normal cross-reacting antigen precursor. Since similarity in protein sequence frequently implies shared function SEQ ID NO: 2 is presumed to share at least some functional similarity with these similar sequences.

A Gap alignment of AAB59513 with SEQ ID NO: 5 revealed that SEQ ID NO: 5 from amino acid 1 to amino acid 722 aligned with AAB59513 from amino acids 20 to 701 with 31% identity and 39% similarity. A Gap alignment of AAC18434 with SEQ ID NO: 5 from amino acids 1 to 387 aligned with AAC18434 from amino acids 74 to 455 with 27% identity and 35% similarity. A Gap alignment of CAA34404 with SEQ ID NO: 5 revealed that SEQ ID NO: 5 from amino acid 1 to 494 aligns with CAA34404 from amino acids 20 to 526 with 31% identity and 36% similarity. A Gap alignment of Q00888 with SEQ ID NO: 5 revealed that SEQ ID NO: 5 from amino acids 1 to 417 aligned with Q00888 from amino acids 535 to 976 with 29% identity and 37% similarity. A Gap alignment of p40199 with SEQ ID NO: 5 revealed that SEQ ID NO: 5 from amino acids 1 to 323 aligned with p40199 from amino acids 20 to 344 with 30% identity and 36% similarity.

The BLASTP analysis also revealed that SEQ ID NO: 5 showed sequence homology to the following proteins: A36319 stated to be carcinoembryonic antigen precursor—human; CAA02706 stated to be unnamed protein product [Homo sapiens]; AAA62835 stated to be carcinoembryonic antigen [Homo sapiens]; P06731 stated to be carcinoembryonic antigen precursor (CEA) (meconium antigen 100) (C D66E ANTIGEN); CAA34474 stated to be pCEA80-11 protein (647 AA) [Homo sapiens]; AAA51826 stated to be biliary glycoprotein I precursor [Homo sapiens]; CAA34404 stated to be TM1-CEA preprotein [Homo sapiens]; Q00888 stated to be pregnancy-specific beta-1-glycoprotein 4 precursor (PSBG-4) (PSBG-9); C30127 stated to be transmembrane carcinoembryonic antigen 3 precursor—human; CAA02704 stated to be unnamed protein product [Homo sapiens]; AAB31183 stated to be BGPc biliary glycoprotein adhesion molecule {alternatively spliced} [human, HT29 colon carcinoma cell line, Peptide, 464aa] [Homo sapiens]; JH0394 stated to be biliary glycoprotein g precursor—human; AAA58394 stated to be biliary glycoprotein a [Homo sapiens]; CAA47697 stated to be biliary glycoprotein [Mus musculus]; S34338 stated to be biliary glycoprotein F—mouse; JC 1509 stated to be biliary glycoprotein E—mouse; A28333 stated to be carcinoembryonic antigen-related protein (clone eLV7)−human (fragment); CAA34405 stated to be TM3-CEA protein [Homo sapiens]; A44783 stated to be ecto-ATPase precursor—rat; S68177 stated to be C-CAM2a protein isoform precursor—rat; AAA16783 stated to be cell adhesion molecule [Rattus norvegicus]; CAA62577 stated to be C-CAM short isoform, C-CAM1 exon 7 missing [Rattus norvegicus]; CAB86230 stated to be carcinoembryonic antigen-related cell adhesion molecule, secreted isoform ceacamla-4C2 [Rattus norvegicus]; P16573 stated to be ecto-atpase precursor (CELL-CAM 105) (C-CAM 105) (atp-dependent taurocolate-carrier protein) (GP110); S23969 stated to be cell-adhesion molecule short form (cell-CAM105)−rat; CAA78054 stated to be S-form Cell-CAM105 isoform(C-CAM2) cloned from rat liver cDNA library [Rattus norvegicus]; AAA37858 stated to be hepatitis virus receptor [Mus musculus]; CAA32940 stated to be TM2-CEA precursor [Homo sapiens]; AAC18439 stated to be BGPi_HUMAN [Homo sapiens]; AAC18439 stated to be BGPi_HUMAN [Homo sapiens]; JH0395 stated to be biliary glycoprotein h precursor—human; P31997 stated to be carcinoembryonic antigen CGM6 precursor (nonspecific cross-reacting antigen NCA-95) (antigen CD67) (CD66B antigen); P40199 stated to be normal cross-reacting antigen precursor (CD66C antigen). Since similarity in protein sequence frequently implies shared function SEQ ID NO: 5 is presumed to share at least some functional similarity with these similar sequences.

A search using HMM and the Pfam database as described above revealed several protein motifs in SEQ ID NO: 5. PF00047 is an immunoglobulin domain motif, found in 9 occurrences within SEQ ID NO: 5 with an overall score of 182.69. The first occurrence within SEQ ID NO: 5 was from amino acids 29 through 102 similar to the model from amino acids 1 through 42; the second match was from SEQ ID NO: 5 amino acids 140 to 197 to the model from amino acids 1 through 45; the third match was from SEQ ID NO: 5 amino acids 232 to 281 to the model from amino acids 1 through 45; the fourth match was from SEQ ID NO: 5 amino acids 357 to 375 to the model from amino acids 27 through 45; the fifth match was from SEQ ID NO: 5 amino acids 410 to 452 to the model from amino acids 1 through 37; the sixth match was from SEQ ID NO: 5 amino acids 620 to 677 to the model from amino acids 1 through 45; the seventh match was from SEQ ID NO: 5 amino acids 719 to 769 to the model from amino acids 1 through 45; the eighth match was from SEQ ID NO: 5 amino acids 808 to 863 to the model from amino acids 3 through 45; the ninth match was from SEQ ID NO: 5 amino acids 905 to 955 to the model from amino acids 1 through 45.

An unannotated Pfamb motif was found to match SEQ ID NO: 5 in three occurrences with a score of 73.70. The first match in SEQ ID NO: 5 was from amino acids 316 to 393 with amino acids 1 to 78 of the Pfamb motif; the second match was from SEQ ID NO: 5 amino acids 618 to 695 to the model from amino acids 1 through 78; the third match was from SEQ ID NO: 5 amino acids 804 to 881 to the model from amino acids 1 through 78. An investigation of the molecules used to generate this motif as described above revealed that it was generated from pregnancy specific glycoprotein beta-1 and its variants, members of the CEA family. The motif was interspersed with the immunoglobulin domains, comparable to its location within SEQ ID NO: 5.

A second unannotated Pfamb motif was found to match SEQ ID NO: 5 with a score of 32.48. SEQ ID NO: 5 from amino acids 12 to 121 aligned with amino acids 1 through 114 of the Pfamb motif. An investigation of the molecules used to generate this motif as described above revealed that it was generated from 46 sequences for a specialized N type immunoglobulin domain found in CEA family members. In most CEA family members the N terminal Ig domain lacks a pair of conserved cysteines, and has been called an N type domain. SEQ ID NO: 5 lacked both cysteines in its N terminal immunoglobulin domain and this motif overlapped the N terminal immunoglobulin domain of SEQ ID NO: 5.

A third unannotated Pfamb was found to match SEQ ID NO: 5 in two occurrences with a score of 19.65. SEQ ID NO: 5 from amino acids 475 to 509 aligned with amino acids 1 through 35 of the Pfamb motif; the second match is from SEQ ID NO: 5 from amino acids 972 to 1004 to amino acids 3 through 35 of the Pfamb motif. An investigation of the molecules used to generate this motif as described above revealed that it was generated from 18 sequences all from CEA family members including: accession P13688, Q15600, P31809, Q03715, P16573, and P40198. The motif flanks and spans the transmembrane domain in all of these molecules, comparable to its second occurrence within SEQ ID NO: 5.

The PeptideStructure program, used as described above, shows a hydrophobic region in SEQ ID NO: 5 centered around amino acid 980 of sufficient size and hydrophobicity that is likely to function as a transmembrane spanning region. As noted above this region demonstrates a shared motif including the transmembrane region and its flanks with members of the CEA family. The sequences N-terminal to these amino acids containing the immunoglobulin domains described above likely constitute an extracellular portion of the molecule. The amino acids C-terminal to this predicted transmembrane domain are likely to form a cytoplasmic domain.

The PeptideStructure program, as described above, also identified a number of potential sites for N-linked glycosylation within the predicted extracellular portion of SEQ ID NO: 5 with strong sites at asparagine residues at amino acids 139, 165, 227, and 274 and a weak site at 176. Members of the CEA family are known to be glycosylated (Paxton et al., PNAS, 84:920-924 (1987)).

Based upon the following two motifs, an N terminal immunoglobulin domain lacking cysteines, multiple extracellular immunoglobulin domains, and the sequence of the encoded protein, SEQ ID NO: 4 encodes a novel member of the CEA family. Due to the dissimilarities between the novel sequences (SEQ ID NOs: 4 and 5) and other CEA family members, there should be no cross-reactivity to known family members at high stringency nucleic acid hybridization. Based upon the pattern of antigenic sites in the polypeptides encoded by the novel polynucleotides, specific antibodies that do not cross-react with known family members can be raised. SEQ ID NOs: 4 and 5 therefore have utility as biomarkers for cancer. Such as prostate cancer and metastesized prostate cancer.

### Example 10

### Exons

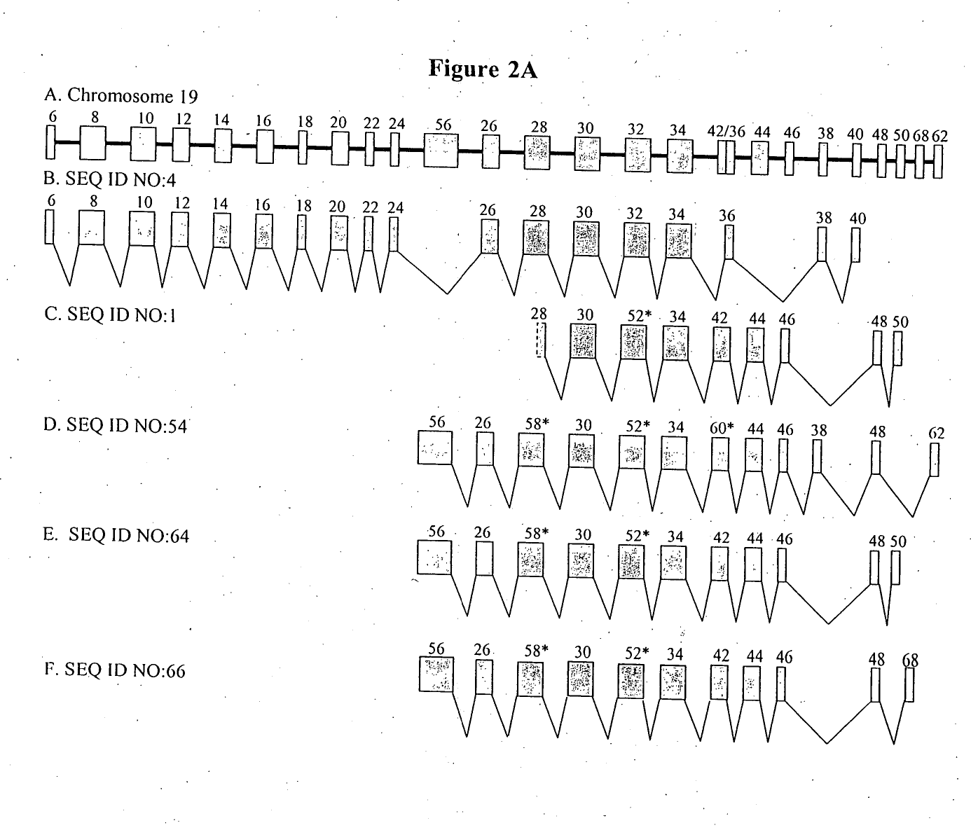

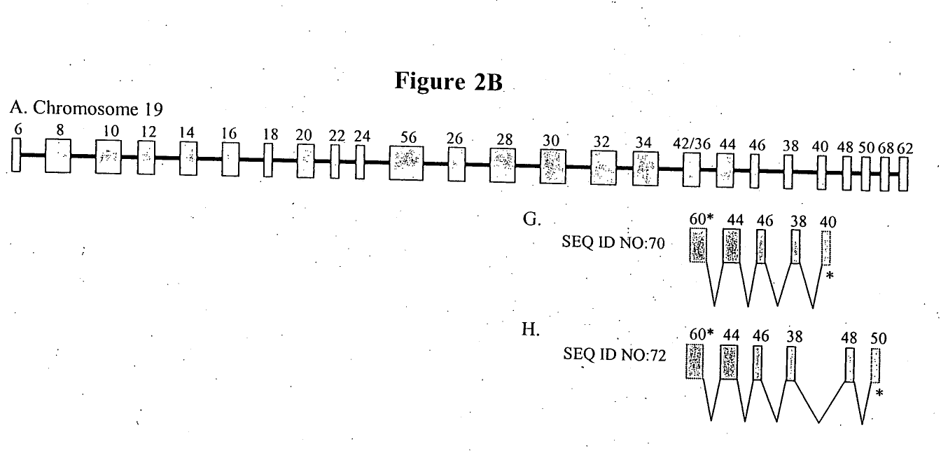

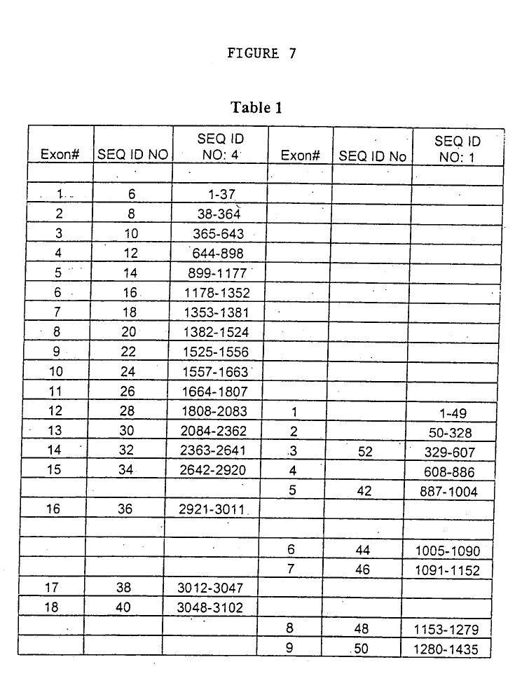

The cDNA sequence of the predicted gene given in SEQ ID NO: 4 comprise 18 exons provided in SEQ ID NO: 6 (last 49 nucleotides), and all of SEQ ID NOs: 8, 10, 12, 14, 16, 18, 20, 22, 24, 26, 28, 30, 32, 34, 36, 38 and 40. The peptides encoded by each of these exons are provided in SEQ ID NOs: 7, 9, 11, 13, 15, 17, 19, 21, 23, 25, 27, 29, 31, 33, 35, 37, 39 and 41, respectively. The cDNA sequence of clone 128375 (SEQ ID NO: 1) comprises 9 exons, provided in SEQ ID NO: 28 (last 49 nucleotides) and in SEQ ID NOs: 30, 34, 42, 44, 46, 50 and 52. The peptide sequences encoded by these exons are provided in SEQ ID NO: 29 and in SEQ ID NOs: 31, 33, 35, 43, 45, 47, 49 and 51, respectively. Each of the exons is given in the order of their occurrence in the gene and with annotations showing their locations in SEQ ID NO: 1 and 4 in Table 1 (FIG. 7) and in FIGS. 2A, 2B fand 2C. Each of the nucleic acid sequences has utility as a biomarker for cancer, since each can be used as a probe to detect the levels of SEQ ID NO: 1, 64 or other splicing variants expressed, for example, in biopsied tissues or postoperatively in excised tumors.

Antigenicity analysis was performed using PlotStructure as described above. The peptides encoded by each exon contained regions of positive antigenicity demonstrating that they were each good substrates for the generation of antibodies. Antibodies to each of these peptides can be used to detect SEQ ID NOs: 2, 3 or 5, in vivo or in vitro.

### Example 11

### Antibody Production to Cell-Adhesion Mediating Polypeptides

To generate antibodies towards a polypeptide of the present invention, the desired peptide is generated using one of the SEQ ID NOs: 1, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 70, 72 and 68. The selected polynucleotide is cloned and the polypeptide is expressed using the CREATOR™ Gene Cloning and Expression System and the PROTM Bacterial Expression System (Clontech Laboratories, Inc., Palo Alto, Calif.) according the manufacturer's instructions. Each polypeptide is be purified, for example, using polyacrylamide gel electrophoresis (Harrington, M G., 1990, Methods in Enzymology, 182:488-495). The purified peptide is then conjugated to to a carrier such as KLH (keyhole limpet hemocyanin) and is used to immunize rabbits.

Serum from the rabbits is tested for reactivity to peptide encoded by the selected polynucleotide using Western blotting. Cell lysates of recombinant cells expressing normal cell lysates of prostate tissue are separated by gel electrophoresis and tranferred to nithocellulose.

Western blot analysis is performed using an affinity purified rabbit anti-peptide antibody adjusted to 2 mg/ml. Blots are incubated for 2 hours at room temperature with the antibodies and then are washed 3 times with tris-buffer. Immunoreactive bands are developed using an anti-rabbit-IgG enzyme conjugated secondary antibody and are visualized by incubation with an appropriate latent chemiluminescent substrate.

Titer is monitored by ELISA to the peptide and by western blotting using the recombinant cell line expressing the peptide. Cross-reactivity is determined and immunoadsorption (using antibodies generated from the remaining above polypeptide sequences) is performed to increase anti-(a) specificity of the polyclonal antibodies when required.

Monoclonal antibodies specific for the selected peptide are generated using hybridoma technology (Hammerling et al., in Monoclonal Antibodies and T-Cell Hybridomas, Elsevier, N.Y., pp. 563-681(1981)). A mouse is immunized with one of the above polypeptides after a purified preparation is obtained from a host cell expression system as described above. The mouse spleen is harvested for splenocytes which are then fused to a suitable myeloma cell line. Hybridoma cells are assayed to identify clones which secrete antibodies capable of specifically binding the polypeptide of the present invention.

### Example 12

### Method of Detecting Abnormal Levels of a Polypeptide in a Biological Sample

The antibodies obtained by the method of the above Example 11 can be used to detect increased or decreased levels of a selected polypeptide in a serum or a biopsy sample from a patient. An antibody-sandwich ELISA is performed by coating the wells of a microtiter plate with antibodies (0.2 to 10 mg/ml) specific to the selected polypeptide. A serial dilution of the serum sample or of the cell lysate of the biopsy is made and a standard dilution curve of recombinantly produced selected polypeptide is also used as a control. Aliquots are allotted to wells coated wells that have also been treated with a blocking agent to reduce non-specific binding, the plate is then incubated for over 2 hours ore more at room temperature. The plate is washed to remove unbound polypeptide.

Alkaline phosphatase conjugated rabbit anti-IgG second antibody is added to each well. The plates are again incubated for over 2 hours at room temperature and washed to remove unbound second antibody. Latent chemiluminescent substrate (4-methylumbelliferyl phosphate or p-nitrophenyl phosphate) is added. The plates are incubated at room temperature and read. Amounts from sample are interpolated using the results from the standard curve.

Alternatively, the wells can be coated with the diluted aliquots of sample and blocked with an appropriate blocking reagent. The bound antigen is then detected by incubating first with an antibody specific for the CEA protein or polypeptide, washed to remove the unbound antibody, and then incubated with a detectably labeled secondary antibody. After washing away the unbound secondary antibody, the bound secondary antibody is detected, thereby detecting the presence or quantity of polypeptide in the sample.

### Example 13

### Microarray Production and Use

MicroArrays are manufactured by spotting polynucleotides of the present invention (for example, a unique 50-90 base pair sequence provided herein) onto conventional silylated glass slides (Cat. No: CSS-25; TeleChem International, Inc.; Sunnyvale, Calif. and Cat. No: 10 484 182, Schleicher & Schuell, Inc., Keene, N.H.). Unique chemiluminescent probes (e.g., labeled with Cy3 or Cy5) are prepared from biopsied tissue both normal and cancerous (late stage) can be used for identification. A hybridization assay is performed and the pattern of expression for each uniquely tagged sequence is analyzed.

Alternatively, polynucleotides of the present invention (one sequence per spot) can be applied to nylon membrane supporting slides or hydrogelstides. Hydrogel slides are cross-linked polyacrylamide gel support slides as described in WOO1/016373, the disclosure of which is incorporated herein by reference in its entirety (Mosaic Technologies, Inc., Waltham, Mass.) that are spotted with Acrydite™ phosphoramidite modified cDNA sequences defined by the exons identified in SEQ ID NOs: 6, 8, 10, 12, 14, 16, 18, 20, 22, 24, 26, 28, 30, 32, 34, 36, 38, 40, 42, 44, 46, 48, 50, 52, 56, 58, 62 and 68 using a MicroGrid, Model BG 600, spotting machine (BioRobotics, Inc.). The phosphoramidite exon sequences are uniquely spotted to known locations. The thiol-derivatized acrylamide gel layer is activated with tris(2-carboxyethyl) phosphine hydrochloride within 30 minutes prior to spotting.

The microarray can be used to identify sequences that specifically hybridize to the known sequences distributed on the array by methods well-known in the art.

### Example 14

### Mammalian Two Hybrid Assay

Using the Clontech Matchmaker™ GAL4 Two-Hybrid System 3 according to the user's manual (PT3247, PR94575, 1999, Clontech Laboratories, Inc.; Palo Alto, Calif.), the polynucleotides of the present invention can be used to screen for proteins that interact with the encoded polypeptides. Generally, the polynucleotide sequences are isolated from the clone 128375 insert, for example, by PCR amplification, using primers designed to amplify the region of interest. The resulting amplified polynucleotide is cleaved with suitable restriction enzymes, and fused into the vectors provided in the kit. This construct is the bait. A prostate tissue library also obtained from Clontech can be used as prey. Protein interactions are assayed following the guidelines provided in Fields and Song, Nature, 340: 245-246 (1989).

Expected results include interactions of the cleaved SEQ ID NO: 1 sequences (or more precisely the polypeptide sequences encoded thereby) with itself and with calmodulin.

### Example 15

### Isolation of Splicing Variants

Additional library screening was conducted to identify cDNA clones comprising splicing variants of SEQ ID NO.1 since the Northern analysis indicated multiple splicing isoforms as discussed above. Probe was made by random priming of SEQ ID NO: 1 using a High Prime DNA labeling kit (No: 1585584, Roche Diagnostics, Indianapolis, Ind.) according to manufacturers instructions. A cDNA library made from human prostate tissue as described above in bacterial hosts was titrated by plating. Approximately 1 million clones were distributed into 96-well plates at a concentration of 1,000 clones per well.

These were grown overnight in Terrific broth (in “Molecular Cloning: A Laboratory Manual,” Sambrook J, Fritsch E F, and Maniatis T, Cold Spring Harbor Laboratory Press (1989)). DNA was prepared from the plasmids using an ATGC Alkaline Lysis Miniprep kit (Edge Biosystems, Gaithersburg, Md.) according to manufacturer's instructions. An aliquot of DNA from each well was transferred to hybridization transfer membrane (Catalog No: NEF9784, NEN, Boston, Mass.) using a 96 pin replicator (Cat No: 250520, Nalge Nunc International). The filters were hybridized to probe overnight under conditions of high stringency at 68° C. in 0.4×White Rain Classic Care Regular Shampoo(Gillette Company, Boston, Mass.). The blots were washed three times in 2×SSC and 0.1% sodium dodecyl sulfate for 20 minutes at room temperature and then three times at 68° C. in 0.1×SSC and 0.1% sodium dodecyl sulfate for 25 minutes each wash.

The filters were exposed to autoradiograms for five days and then developed. DNA corresponding to wells having a positive signal was taken from the original 96-well plate for every well that gave a positive signal. This DNA was electroporated into bacterial hosts using standard methods (in “Molecular Cloning: A Laboratory Manual,” Sambrook J, Fritsch E F, and Maniatis T, Cold Spring Harbor Laboratory Press (1989)). The bacteria were then dispensed into new 96-well plates at a concentration of 50 clones per well. They were processed as described in the preceding paragraph. Autoradiograms were exposed to these filters overnight. DNA corresponding to wells having a positive signal was electroporated into bacterial hosts. These bacteria were then plated on Luria Broth agar plates prepared by dissolving 20 g of Bacto Luria Broth, Lennox (Cat. No: 0402-08-0, Becton Dickinson and Company, Franklin Lakes, N.J.), and 15 g of Bacto-Agar (Cat. No: 0140-01, Becton Dickinson and Company, Franklin Lakes, N.J.) per liter of distilled water and containing 100 micrograms per milliliter of carbenicillin (Cat. No: C-1389, Sigma, St. Louis, Mo.). Colonies were grown for 20 hours at 37° C. Colonies were picked individually into wells of new 96-well plates. These plates were then processed as described in the preceding paragraph. The autoradiograms were exposed overnight and DNA from all positive wells was electroporated into bacterial host. These bacteria were then plated as described above and sequencing was accomplished as described in Example 2.

### Example 16

### Clone PCEA2

Clone PCEA2 (SEQ ID NO: 54) was identified from a cDNA library created from human prostate tissue as described in Example 1. Clone PCEA2 was selected from this library based upon cross-hybridization with SEQ ID NO: 1 as described above in Example 15.

The EcoRI/NotI restriction fragment insert is about 2147 base pairs. A complete open reading frame is present with a starting methionine and a stop codon. This open reading frame begins at nucleotide 313 and ends at nucleotide 2067 with a stop codon from nucleotides 2068 through 2070. This sequence encodes a polypeptide that is 585 amino acids in length. The deduced amino acid sequence encoded by this nucleotide sequence is shown in SEQ ID NO: 55.

A “BLASTN” analysis of SEQ ID NO: 54 showed no significant homology to any expressed human nucleotide sequences, only to human genomic DNA sequence. Genomic BAC clones, GenBank accession numbers AC073898 and AC069278, have regions of exact matches to SEQ ID NO: 54. These BAC clones are stated to be from chromosome 19.

A “BLASNP” analysis of SEQ ID NO: 55 showed greatest sequence homology to a protein sequence having GenBank accession number BAB31307, that was also referred to as murine adult male cecum cDNA, the nucleotide sequence of which is provided in GenBank accession number AK018613. A GAP alignment of BAB31307 with SEQ ID NO: 55 revealed that SEQ ID NO: 55 from amino acids 1 to 585 aligned with BAB31307 from amino acids 1 to 573 with 57% identity and 60% similarity.

The “BLASTP” analysis further revealed that SEQ ID NO: 55 showed sequence homology to many proteins of the CEA family including GenBank accession number AAA51967 which is stated to be human carcinoembryonic antigen mRNA (CEA), SwissProt accession number PSG4_HUMAN stated to be human pregnancy-specific beta-1-glycoprotein 4 precursor, and GenBank accession number AAA51826 stated to be biliary glycoprotein I precursor (Homo sapiens). Since similarity in protein sequence suggests shared function SEQ ID NO: 55 is presumed to share at least some functional similarity with these similar sequences.

A Gap alignment of AAA51967 with SEQ ID NO: 55 revealed that SEQ ID NO: 55 from amino acid 1 to amino acid 462 aligned with AAA51967 from amino acids 244 to 702 with 33% identity and 40% similarity. A Gap alignment of PSG4_HUMAN with SEQ ID NO: 55 from amino acids 1 to 438 aligned with PSG4_HUMAN from amino acids 1 to 419 with 32% identity and 40% similarity. A Gap alignment of AAA51826 with SEQ ID NO: 55 revealed that SEQ ID NO: 55 from amino acid 1 to 440 aligns with AAA51826 from amino acids 92 to 526 with 32% identity and 39% similarity.

The “BLASTP” analysis also revealed that SEQ ID NO: 55 showed sequence homology to the following proteins: CAA34405 stated to be TM3-CEA protein [Homo sapiens]; CAA34404 stated to be TM1-CEA [Homo sapiens]; CAA02706 stated to be an unnamed protein product [Homo sapiens]; AAA62835 stated to be carcinoembryonic antigen [Homo sapiens]; AAA51963 stated to be carcinoembryonic antigen precursor [Homo sapiens]; CAA34474 stated to be pCEA80-11 protein (647 AA) [Homo sapiens]; PSG3_Human stated to be Pregnancy-specific beta-1-glycoprotein 3 precursor (PSBG-3) (carcinoembryonic antigen SG5); AAC60584 stated to be pregnancy-specific beta 1-glycoprotein, PSG {clone hIS25} [human, colon, Peptide, 428 aa] [Homo sapiens]; CAA01646 stated to be trophoblast membrane expressed protein [Homo sapiens]; AAA52606 stated to be pregnancy-specific beta-1 glycoprotein precursor [Homo sapiens]; AAA60960 stated to be carcinoembryonic antigen SG9 [Homo sapiens]; AAA60204 stated to be fetal liver non-specific cross-reactive antigen precursor protein [Homo sapiens]; AAA60203 stated to be PSG11 [Homo sapiens]; CAA35612 stated to be pregnancy-specific beta-1 glycoprotein (AA 1-426) [Homo sapiens]; B35334 stated to be pregnancy-specific beta-1-glycoprotein 7 precursor; AAC18437 stated to be biliary glycoprotein g precursor; C30127 stated to be transmembrane carcinoembryonic antigen 3 precursor—human; and S34338 stated to be biliary glycoprotein F—mouse and P16573 (ECTO_RAT) stated to be ECTO_ATPase precursor (CELL-CAM 105) (C-CAM 1 05) (atp-dependent taurocolate-carrier protein) (GP110).

A search using HMM and the Pfam database as described above revealed several protein motifs in SEQ ID NO: 55. The imumunoglobulin domain model PF00047 was found to occur four times within SEQ ID NO: 55 with an overall matching score of 102.18. The first occurrence of the immunoglobulin domain within SEQ ID NO: 55 is from amino acids 88 through 140 similar to the PF00047 model from amino acids 1 through 45. The second match is from SEQ ID NO: 55 amino acids 182 through 232 to the PF00047 model from amino acids 1 through 45. The third match is from SEQ ID NO: 55 amino acids 271 through 326 to the PF00047 model from amino acids 3 through 45. The last is from SEQ ID NO: 55 amino acids 368 through 418 to the PF00047 model from amino acids 1 through 45.

An unannotated Pfamb motif was found to match SEQ ID NO: 55 twice with an overall score of 60.96. SEQ ID NO: 55 from amino acids 72 to 157 aligned with amino acids 2 to 87 of the Pfamb motif, and SEQ ID NO: 55 from amino acids 257 to 343 aligned with amino acids 1 through 87 of the Pfamb motif. An investigation of the molecules used to generate this motif as described above revealed that it was generated from pregnancy specific glycoprotein beta-1 and its variants, members of the CEA family. The motif was interspersed with the immunoglobulin domains, comparable to its location within SEQ ID NO: 55.

A second unannotated Pfamb was found to match SEQ ID NO: 55 with a score of 31.63. SEQ ID NO: 55 from amino acids 441 to 476 aligned with amino acids 1 through 36 of the Pfamb motif. As described above, this motif from 18 sequences all from CEA family members. The motif flanks and spans the transmembrane domain in all of these molecules, comparable to its location within SEQ ID NO: 55.

The PeptideStructure program, used as described above, showed a hydrophobic region in SEQ ID NO: 55 centered around amino acid 457 of sufficient size and hydrophobicity that is likely to function as a transmembrane spanning region. As noted above, this region demonstrates a shared motif including the transmembrane region and its flanking sequence with members of the CEA family. The sequences N-terminal to these amino acids containing the immunoglobulin domains, described above, likely constitute an extracellular portion of the molecule. The amino acids C-terminal to this predicted transmembrane domain are likely to form a cytoplasmic domain.

The PeptideStructure program, as described above, also identified a number of potential sites for N-linked glycosylation within the predicted extracellular portion of SEQ ID NO: 55 with strong sites at asparagine residues at amino acids 96, 105, 280, 306, 368 and 415 and a weak site at 317. Members of the CEA family are known to be glycosylated (Paxton et al., PNAS, 84:920-924 (1987)).

The PeptideSort program, as described above, showed that SEQ ID NO: 55 had a molecular weight of 64,501 Daltons and an isoelectric point of 5.74. The cytoplasmic domains of CEA family members human biliary glycoprotein (CEACAM1) and mouse homologs C-CAM1 and C-CAM2 contain binding sites for calmodulin. All three of these molecules share a calmodulin-binding site in the cytoplasmic domain adjacent to the transmembrane domain (Edlund et al., J Biol Chem, 271:1393-1399). SEQ ID NO: 55 shared some sequence conservation from amino acid 468 through 481 ‘FLCIRNARRPSRKT’ (SEQ ID NO: 80) including two charged amino acids at 479 and 480. Both murine homologs contain a second calmodulin-binding site closer to the C-terminus of the cytoplasmic tail that was not found in human biliary glycoprotein. A minimal calmodulin-binding motif ‘Hydrophobic-Q-X3-R’ (Aitken, Molecular Biotechnology, 12:241-53 (1999)) was found in a comparable location in SEQ ID NO: 55 from amino acids 517 to 522 ‘LQGRIR’ (SEQ ID NO: 75). Human biliary glycoprotein forms homodimers and this process is regulated by calmodulin (Edlund et al., J Biol Chem, 271:1393-1399). A similar process may be inferred from sequence similarity and binding motifs found in SEQ ID NO: 55.

The serine found at amino acid 551 of SEQ ID NO: 55 matched the consensus for phosphorylation targets of proline-directed cell-cycle kinases ‘S/T-P-X-K/R’ (Aitken, 1999, ibid) having ‘SPWK’ (SEQ ID NO: 76) from amino acids 551 through 554. SEQ ID NO: 55 also had two matches to the consensus motif ‘Y-X-X-hydrophobic’ to which SH2 domains can bind when the tyrosine is phosphorylated (Aitken, 1999, ibid). These motifs were found from amino acids 511 through 514 ‘YCNI’ (SEQ ID NO: 77) and from amino acids 578 through 581 ‘YEVL’ (SEQ ID NO: 81).

A Gap alignment of SEQ ID NO: 54 to SEQ ID NO: 1 revealed that SEQ ID NO: 54 had 735 nucleotides at the 5′ end not found in SEQ ID NO: 1. SEQ ID NO: 54 from nucleotides 736 to 1887 aligned with SEQ ID NO: 1 from nucleotides 1 to 1152 nearly 100% identity having only a single nucleotide difference at position 1721 where SEQ ID NO: 54 has a guanine and SEQ ID NO: 1 has a cytosine at corresponding position 986. SEQ ID NO: 54 had an insertion from nucleotides 1888 to 1923 between nucleotides 1152 an 1153 of SEQ ID NO: 1. SEQ ID NO: 54 from nucleotides 1924 to 2049 aligned to SEQ ID NO: 1 from 1153 to 1278 with 100% identity. SEQ ID NO: 54 from nucleotides 2050 to 2147 had little homology to SEQ ID NO: 1 from nucleotides 1279 to 1435.

A Gap alignment of SEQ ID NO: 55 to SEQ ID NO: 2 revealed that SEQ ID NO: 55 was longer than SEQ ID NO: 2 on the amino terminus having 179 additional amino acids not found in SEQ ID NO: 2. SEQ ID NO: 55 aligned from amino acid 180 to 507 to SEQ ID NO: 2 from amino acids 1 to 346 exactly with a single amino acid difference at position 470 where SEQ ID NO: 55 had a cysteine and SEQ ID NO: 2 had a tyrosine at the corresponding amino acid 291. SEQ ID NO: 55 had an insertion from amino acids 526 to 537 between amino acids 436 and 347 of SEQ ID NO: 2. SEQ ID NO: 55 from amino acids 538 to 579 aligned with SEQ ID NO: 2 from amino acids 347 to 387 with 100% identity. SEQ ID NO: 55 contained an additional 6 amino acids from 580 to 585 with little identity to SEQ ID NO: 2 from 388 to 405.

A Gap alignment of SEQ ID NO: 54 to SEQ ID NO: 4 revealed that SEQ ID NO: 54 from 1 to 364 had little homology to SEQ ID NO: 4 from nucleotides 1 to 1663. SEQ ID NO: 54 from 365 to 1623 aligned with SEQ ID NO: 4 from 1664 to 2920 with nearly 100% identity having only 2 nucleotide differences where SEQ ID NO: 54 at nucleotide 634 has an adenine and SEQ ID NO: 4 has a guanine at corresponding nucleotide 1933 and where SEQ ID NO: 54 at nucleotide 1269 has a cytosine and SEQ ID NO: 4 has a guanine at corresponding nucleotide 2568. SEQ ID NO: 54 had an insertion from nucleotides 1622 to 1648 between corresponding nucleotides 2920 and 2921 of SEQ ID NO: 4. SEQ ID NO: 54 from nucleotides 1649 to 1739 aligned with SEQ ID NO: 4 from 2921 to 3011.

A Gap alignment of SEQ ID NO: 55 to SEQ ID NO: 5 revealed that SEQ ID NO: 55 was shorter than SEQ ID NO: 5 on the amino terminus having 18 amino acids with little homology to amino acids 1 through 555 of SEQ ID NO: 5. SEQ ID NO: 55 aligned from amino acid 19 to 436 to SEQ ID NO: 5 from amino acids 556 to 973 exactly with a single amino acid difference at 108 where SEQ ID NO: 55 had an isoleucine and SEQ ID NO: 5 had a valine at the corresponding amino acid 645. SEQ ID NO: 55 was found to have a small insertion from amino acids 437 through 448 between amino acids 973 and 974 of SEQ ID NO: 5. SEQ ID NO: 55 from amino acids 449 through 476 then matched exactly to SEQ ID NO: 5 from amino acids 974 to 1004 with a single amino acid difference where SEQ ID NO: 55 had a cysteine at position 470 and SEQ ID NO: 5 had a tyrosine at the corresponding amino acid 998. SEQ ID NO: 55 from amino acids 477 to 585 had little homology to SEQ ID NO: 5 from amino acids 1005 to 1033.

CEA family members exhibit a characteristic pattern of immunoglobulin domain distribution. SEQ ID NO: 55 has half of an N-terminal V-type immunoglobulin domain, and four C-type immunoglobulin domains, of alternating A and B subtypes. An N-terminal Ig domain followed by alternating A and B subtypes Ig domains is characteristic of the CEA family. A comparison of the domain structure of SEQ ID NO: 55 with a known CEA family member CEACAM1 is given in FIG. 3.

Based upon the similarity of protein sequence to other CEA molecules, shared HMM motifs, similar patterns of Ig domain distribution, localization to chromosome 19 and calmodulin binding motifs, SEQ ID NO: 54 encodes a novel member of the CEA family. SEQ ID NO: 55 is a novel member of the CEA family. Other members of the CEA family are known to have altered levels of expression in numerous cancers (review, Hammerstrom ibid). Thus, SEQ ID NO: 54 and its expressed polypeptide SEQ ID NO: 55 are useful as tumor markers and markers for metastasized prostate tissue. However, even absent differential expression in tumors, a polyp eptide or polynucleotide is useful as a tumor marker when it shows tissue specificity. Some CEA family members have been proven useful for immunolocalization of tumor tissue (e.g., Nakopoulou et al., Dis Colon Rectum, 26:269-74 (1983)), in particular for radioimmunosurgery (Bertoglio et al., Seminars in Surgical Oncology, and for immunotherapy (Khare et al., Cancer Research, 61:370-5 (2001); Buchegger et al., Int J Cancer, 41:127-134 (1988)).

While SEQ ID NOs: 54 and 55 share sequence similarities to other CEA family members, cross-reactivity to known family members is expected under conditions of high stringency nucleic acid hybridization due to the extent of unique sequence. Specific antibodies that do not cross-react with known family members can be raised based upon the pattern of antigenic sites present in the polypeptides encoded by the polynucleotide in cDNA SEQ ID NO: 54.

### Example 17

### Exon Structure of Clone PCEA2

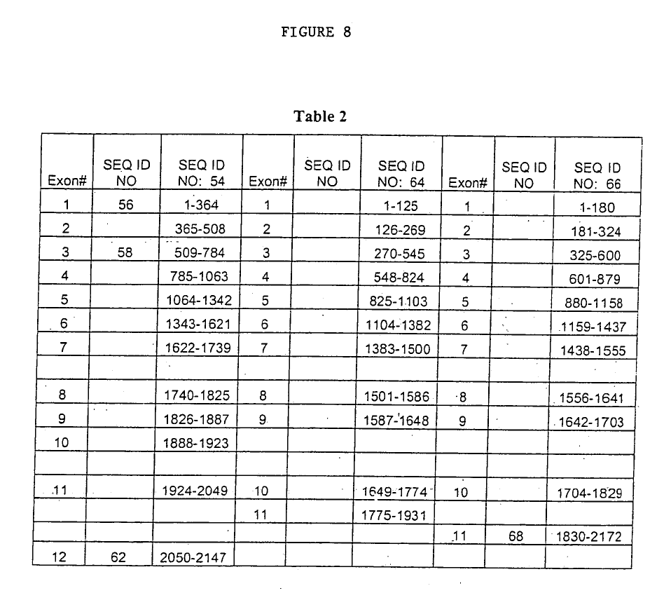

The exon structure of SEQ ID NO: 54 is diagramed in FIG. 2D, and shown in Table 2 (FIG. 8). The cDNA sequence given in SEQ ID NO: 54 is comprised of 12 exons, SEQ ID NOs: 56, 26, 30, 52, 34, 60, 44, 46, 38, 48 and 62. SEQ ID NO: 52 differs from SEQ ID NO: 32 by a single nucleotide. SEQ ID NO: 60 differs from SEQ ID NO: 42 by a single nucleotide.

SEQ ID NOs: 60 and 62 are exons unique to splice variant SEQ ID NO: 54 and have utility as biomarkers for cancer, since each can be used as a probe to detect the levels of SEQ ID NO: 54 expressed in biopsied tissues or postoperatively in excised tumors.

The peptides encoded by each of these exons are SEQ ID NOs: 57, 27, 59, 31, 33, 35, 61, 45, 47, 53, 49, and 63, respectively. Antigenicity analysis was performed using PlotStructure as described above. The peptides encoded by each exon contained regions of positive antigenicity demonstrating that they were each good substrates for the generation of antibodies. Antibodies to each of these peptides can be used to detect SEQ ID NO: 55 in tissue in vivo or in vitro.

### Example 18

### Clone PCEA1-FL

Clone PCEA1-FL (SEQ ID NO: 64) was identified from a cDNA library created from human prostate tissue as described in Example 1. Clone PCEA1-FL was selected from this library based upon cross-hybridization with SEQ ID NO: 1 as described above in Example 15.

The insert is about 1931 base pairs. The nucleotide sequence of this insert is represented as SEQ ID NO: 64. A complete open reading frame is present with a starting methionine and a stop codon. This open reading frame begins at nucleotide 74 and ends at nucleotide 1825 with a stop codon from nucleotides 1826 through 1828. This sequence encodes a polypeptide that is 585 amino acids in length. The deduced amino acid sequence encoded by this nucleotide sequence is shown in SEQ ID NO: 65.

A “BLASTN” analysis of SEQ ID NO: 64 showed no significant homology to any expressed human nucleotide sequences, only to human genomic DNA sequence in Genomic BAC clones, GenBank accession numbers AC073898 and AC069278.

A “BLASTP” analysis of SEQ ID NO: 65 showed greatest sequence homology to a protein sequence having GenBank accession number BAB31307, that was also referred to as murine adult male cecum cDNA, the nucleotide sequence of which is provided in GenBank accession number AK018613. A Gap alignment of BAB31307 with SEQ ID NO: 65 revealed that SEQ ID NO: 65 from amino acids 1 to 584 aligned with BAB31307 from amino acids 1 to 577 with 56% identity and 60% similarity.

The “BLASTP” analysis further revealed that SEQ ID NO: 65 showed sequence homology to many proteins of the CEA family including GenBank accession number AAA51967 which is stated to be human carcinoembryonic antigen mRNA (CEA), SwissProt accession number PSG4_HUMAN stated to be human pregnancy-specific beta-1-glycoprotein 4 precursor, and GenBank accession number AAA51826 stated to be biliary glycoprotein I precursor (Homo sapiens). Since similarity in protein sequence suggests shared function SEQ ID NO: 65 is presumed to share at least some functional similarity with these similar sequences.

A Gap alignment of AAA51967 with SEQ ID NO: 65 revealed that SEQ ID NO: 65 from amino acid 1 to amino acid 462 aligned with AAA51967 from amino acids 240 to 701 with 33% identity and 40% similarity. A Gap alignment of PSG4_HUMAN with SEQ ID NO: 65 from amino acids 1 to 438 aligned with PSG4_HUMAN from amino acids 1 to 419 with 32% identity and 40% similarity. A Gap alignment of AAA51826 with SEQ ID NO: 65 revealed that SEQ ID NO: 65 from amino acid 1 to 439 aligns with AAA51826 from amino acids 92 to 526 with 32% identity and 39% similarity.

The “BLASTP” analysis also revealed that SEQ ID NO: 65 showed sequence homology to the following proteins: CAA34405 stated to be TM3-CEA protein [Homo sapiens]; CAA34404 stated to be TM1-CEA [Homo sapiens]; CAA02706 stated to be an unnamed protein product [Homo sapiens]; AAA62835 stated to be carcinoembryonic antigen [Homo sapiens]; AAA51963 stated to be carcinoembryonic antigen precursor [Homo sapiens]; CAA34474 stated to be pCEA80-11 protein (647 AA) [Homo sapiens]; PSG3_Human stated to be pregnancy-specific beta 1-glycoprotein 3 pecursor (PSBG-3) (carcinoembryonic antigen SG5); AAC60584 stated to be pregnancy-specific beta 1-glycoprotein, PSG {clone hIS25} [human, colon, Peptide, 428 aa] [Homo sapiens]; CAA01646 stated to be trophoblast membrane expressed protein [Homo sapiens]; AAA52606 stated to be pregnancy-specific beta-1 glycoprotein precursor [Homo sapiens]; AAA60960 stated to be carcinoembryonic antigen SG9 [Homo. sapiens]; AAA60204 stated to be fetal liver non-specific cross-reactive antigen precursor protein [Homo sapiens]; AAA60203 stated to be PSG11 [Homo sapiens]; CAA35612 stated to be pregnancy-specific beta-1 glycoprotein (AA 1-426) [Homo sapiens]; B35334 stated to be pregnancy-specific beta-1-glycoprotein 7 precursor; AAC18437 stated to be biliary glycoprotein g precursor; C30127 stated to be transmembrane carcinoembryonic antigen 3 precursor—human; and S34338 stated to be biliary glycoprotein F—mouse and P16573 (ECTO_RAT) stated to be ECTO_ATPase precursor (CELL-CAM 105) (C-CAM 1 05) (ATP-dependent taurocolate-carrier protein) (GP110).

A search using HMM and the Pfam database as described above revealed several protein motifs in SEQ ID NO: 65. The immunoglobulin domain model PF00047 was found to occur four times within SEQ ID NO: 65 with an overall matching score of 102.18. The first occurrence of the immunoglobulin domain within SEQ ID NO: 65 is from amino acids 83 through 140 similar to the PF00047 model from amino acids 1 through 44. The second match is from SEQ ID NO: 65 amino acids 182 through 232 to the PF00047 model from amino acids 1 through 45. The third match is from SEQ ID NO: 65 amino acids 271 through 326 to the PF00047 model from amino acids 3 through 45. The last is from SEQ ID NO: 65 amino acids 368 through 418 to the PF00047 model from amino acids 1 through 45.

An unannotated Pfamb motif was found to match SEQ ID NO: 65 twice with an overall score of 60.96. SEQ ID NO: 65 from amino acids 72 to 157 aligned with amino acids 2 to 87 of the Pfamb motif, and SEQ ID NO: 65 from amino acids 257 to 343 aligned with amino acids 1 through 87 of the Pfamb motif. An investigation of the molecules used to generate this motif as described above revealed that it was generated from pregnancy specific glycoprotein beta-1 and its variants, members of the CEA family. The motif was interspersed with the immunoglobulin domains, comparable to its location within SEQ ID NO: 65.

A second unannotated Pfamb was found to match SEQ ID NO: 65 with a score of 35.10. SEQ ID NO: 65 from amino acids 441 to 476 aligned with amino acids 1 through 36 of the Pfamb motif. An investigation of the molecules used to generate this motif as described above revealed that it was generated from 18 sequences all from CEA family members including: GenBank accession numbers P13688 stated to be human biliary glycoprotein 1 precursor; Q15600 stated to be TM2-CEA precursor; P31809 stated to be murine biliary glycoprotein 1 precursor; Q03715 stated to be nonspecific cross-reacting antigen; P16573 stated to be rat ecto-ATPase precursor; and P40198 stated to be human carcinoembryonic antigen CGM1 precursor. The motif flanks and spans the transmembrane domain in all of these molecules, comparable to its location within SEQ ID NO: 65.

The PeptideStructure program, used as described above, showed a hydrophobic region in SEQ ID NO: 65 centered around amino acid 457 of sufficient size and hydrophobicity that is likely to function as a transmembrane spanning region. As noted above this region demonstrates a shared motif including the transmembrane region and its flanking sequence with members of the CEA family. The sequences N-terminal to these amino acids containing the immunoglobulin domains, described above, likely constitute an extracellular portion of the molecule. The amino acids C-terminal to this predicted transmembrane domain are likely to form a cytoplasmic domain.

The PeptideStructure program, as described above, also identified a number of potential sites for N-linked glycosylation within the predicted extracellular portion of SEQ ID NO: 65 with strong sites at asparagine residues at amino acids 96, 105, 280, 306, 368, 415 and 513 and weak sites at 317 and 581.

The PeptideSort program, as described above, showed that SEQ ID NO: 65 had a molecular weight of 64,383.36 Daltons and an isoelectric point of 5.95. The cytoplasmic domains of CEA family members human biliary glycoprotein (CEACAM1) and mouse homologs C-CAM1 and C-CAM2 contain binding sites for calmodulin. All three of these molecules share a calmodulin-binding site in the cytoplasmic domain adjacent to the transmembrane domain (Edlund et al., J Biol Chem, 271:1393-1399). SEQ ID NO: 65 shared some sequence conservation in this region from amino acid 468 through 481 ‘FLYIRNARRPSRKT’ (SEQ ID NO: 74) including two charged amino acids at 479 and 480. Both murine homologs contain a second calmodulin-binding site closer to the C-terminus of the cytoplasmic tail that was not found in human biliary glycoprotein. A minimal calmodulin-binding motif ‘Hydrophobic-Q-X3-R’ (Aitken, Molecular Biotechnology, 12:241-53 (1999)) was found in a comparable location in SEQ ID NO: 65 from amino acids 517 to 522 ‘LQGRIR’ (SEQ ID NO: 75). Human biliary glycoprotein forms homodimers and this process is regulated by calmodulin (Edlund et al., J Biol Chem, 271:1393-1399). A similar process may be inferred from sequence similarity and binding motifs found in SEQ ID NO: 65

The serine found at amino acid 539 of SEQ ID NO: 65 matched the consensus for phosphorylation targets of proline-directed cell-cycle kinases ‘S/T-P-X-K/R’ (Aitken, 1999, ibid) having ‘SPWK’ (SEQ ID NO: 76) from amino acids 539 through 542. SEQ ID NO: 65 also had three matches to the consensus motif ‘Y-X-X-hydrophobic’ to which SH2 domains can bind when the tyrosine is phosphorylated (Aitken, 1999, ibid). These motifs were found from amino acids 511 through 514 ‘YCNI’, (SEQ ID NO: 77) amino acids 566 through 569 ‘YEEL’ (SEQ ID NO: 78) and from amino acids 577 through 580 ‘YIQI’ (SEQ ID NO: 79).

A Gap alignment of SEQ ID NO: 64 to SEQ ID NO: 1 revealed that SEQ ID NO: 64 had 496 nucleotides at the 5′ end not found in SEQ ID NO: 1. SEQ ID NO: 64 from nucleotides 497 to 1931 aligned with SEQ ID NO: 1 from nucleotides 1 to 1435 at 100% identity.

A Gap alignment of SEQ ID NO: 65 to SEQ ID NO: 2 revealed that SEQ ID NO: 65 was longer than SEQ ID NO: 2 on the amino terminus having 179 additional amino acids not found in SEQ ID NO: 2. SEQ ID NO: 65 aligned from amino acid 180 to 584 to SEQ ID NO: 2 exactly.

A Gap analysis of SEQ ID NO: 64 to SEQ ID NO: 4 revealed that SEQ ID NO: 64 from 1 to 126 had little homology to SEQ ID NO: 4 from nucleotides 1 to 1663. SEQ ID NO: 64 from 127 to 1381 aligned with SEQ ID NO: 4 from 1664 to 2918 with nearly 100% identity having only two nucleotide differences where SEQ ID NO: 64 at nucleotide 395 has an adenine and SEQ ID NO: 4 has a guanine at corresponding nucleotide 1933 and where SEQ ID NO: 64 at nucleotide 1030 has a cytosine and SEQ ID NO: 4 has a guanine at corresponding nucleotide 2568. SEQ ID NO: 64 had an insertion from nucleotides 1383 to 1409 between corresponding nucleotides 2920 and 2921 of SEQ ID NO: 4. SEQ ID NO: 64 from nucleotides 1410 to 1500 aligned with SEQ ID NO: 4 from 2921 to 3011.

A Gap analysis of SEQ ID NO: 65 to SEQ ID NO: 5 revealed that SEQ ID NO: 65 was shorter than SEQ ID NO: 5 on the amino terminus having 18 amino acids with little homology to amino acids 1 through 555 of SEQ ID NO: 5. SEQ ID NO: 65 then aligned from amino acid 19 to 436 to SEQ ID NO: 5 from amino acids 556 to 973 exactly with a single amino acid difference at 108 where SEQ ID NO: 65 had an isoleucine and SEQ ID NO: 5 had a valine at the corresponding amino acid 645. SEQ ID NO: 65 was found to have a small insertion from amino acids 437 through 445 between amino acids 973 and 974 of SEQ ID NO: 5. SEQ ID NO: 65 from amino acids 446 through 476 then matched exactly to SEQ ID NO: 5 from amino acids 974 to 1004. SEQ ID NO: 65 from amino acids 477 to 509 had little homology to SEQ ID NO: 5 from amino acids 1005 to 1033.

A Gap alignment of SEQ ID NO: 64 to SEQ ID NO: 54 revealed that SEQ ID NO: 64 from nucleotides 2 to 1648 aligned with SEQ ID NO: 54 from nucleotides 2 to 1887 nearly 100% identity having only a single nucleotide difference at position 1482 where SEQ ID NO: 64 has a adenosine and SEQ ID NO: 54 has a guanine at corresponding position 1721. SEQ ID NO: 64 has no homology to SEQ ID NO: 54 from nucleotides 1888-1923. SEQ ID NO: 64 from nucleotides 1649 to 1775 aligned to SEQ ID NO: 54 from 1924 to 2049 with 100% identity. SEQ ID NO: 64 from nucleotides 1776 to 1873 had little homology to SEQ ID NO: 54 from nucleotides 2050 to 2147.

A Gap alignment of SEQ ID NO: 65 to SEQ ID NO: 55 revealed that SEQ ID NO: 65 aligned from amino acid 1 to 525 to SEQ ID NO: 54 from amino acids 1 to 525 exactly. SEQ ID NO: 65 no homology from amino acids 525 to 526 between amino acids 525 and 537 of SEQ ID NO: 54. SEQ ID NO: 65 from amino acids 526 to 567 aligned with SEQ ID NO: 54 from amino acids 537 to 579 with 100% identity. SEQ ID NO: 65 had little homology from 568 to 584 with little identity to SEQ ID NO: 54 from 580 to 585.

SEQ ID NO: 65 has half of an N-terminal V-type immunoglobulin domain, and then four C-type immunoglobulin domains, of alternating A and B subtypes. An N-terminal Ig domain followed by alternating A and B subtype Ig domains is characteristic of the CEA family. A comparison of the domain structure of SEQ ID NO: 65 with a known CEA family member CEACAM1 is given in FIG. 3.

Based upon the similarity of protein sequence to other CEA molecules, shared HMM motifs, similar patterns of Ig domain distribution, localization to chromosome 19 and calmodulin binding motifs, SEQ ID NO: 64 encodes a novel member of the CEA family. SEQ ID NO: 65 is novel member of the CEA family. SEQ ID NO: 64 and its expressed polypeptide SEQ ID NO: 65 are useful as tumor markers. However, even absent differential expression in tumors, a polypeptide or polynucleotide is useful as a tumor marker when it shows tissue specificity.

While SEQ ID NO: 64 and 65 share sequence similarities to other CEA family members, no cross-reactivity to known family members is expected under conditions of high stringency nucleic acid hybridization due to the extent of unique sequence. Specific antibodies that do not cross-react with known family members can be raised based upon the pattern of antigenic sites present in the polypeptides encoded by the polynucleotide in cDNA SEQ ID NO: 64. Since SEQ ID NO: 64 was isolated from human prostate tissue, shows strong expression in that tissue, and was isolated as a variant of SEQ ID NO: 1, 64 and the polypeptide it encodes SEQ ID NO: 65 are useful as biomarkers of prostate tissue and as markers for metastasized prostate tissue.

### Example 19

### Exon Structure of Clone PCEA1-FL

The exon structure of SEQ ID NO: 64 is diagramed in FIG. 2E and shown in Table 2, above. The cDNA sequence is SEQ ID NO: 64 and comprises 11 exons: SEQ ID NOs: 56, 26, 58, 30, 52, 34, 42, 44, 46, 48 and 50. SEQ ID NO: 52 differs from SEQ ID NO: 32 by a single nucleotide.

SEQ ID NO: 50 is an exon unique to SEQ ID NO: 64 and as utility as a biomarker for cancer, since it can be used as a probe to detect the levels of SEQ ID NO: 64 expressed in biopsied tissues or postoperatively in excised tumors.

The peptides encoded by each of these exons are SEQ ID NOs: 57, 27, 59, 31, 33, 35, 43, 45, 47, 49 and 51, respectively. Antigenicity analysis was performed using PlotStructure as described above. The peptides encoded by each exon contained regions of positive antigenicity demonstrating that they were each good substrates for the generation of antibodies. Antibodies to each of these peptides can be used to detect SEQ ID NO: 65 in tissue in vivo or in vitro.

### Example 20

### Clone PCEA3

Clone PCEA3 (SEQ ID NO: 66) was identified from a cDNA library created from human prostate tissue as described in Example 1. Clone PCEA3 was selected from this library based upon cross-hybridization with SEQ ID NO: 1, as described above in Example 15.

The insert is about 2172 base pairs. A complete open reading frame is present with a starting methionine and a stop codon. This open reading frame begins at nucleotide 129 and ends at nucleotide 1862 with a stop codon from nucleotides 1863 through 1865. This sequence encodes a polypeptide that is 578 amino acids in length. The deduced amino acid sequence encoded by this nucleotide sequence is shown in SEQ ID NO: 67.

A “BLASTN” analysis of SEQ ID NO: 66 showed no significant homology to any expressed human nucleotide sequences, only to human genomic DNA sequence present in BAC clones, GenBank accession numbers AC073898 and AC069278.

A “BLASTP” analysis of SEQ ID NO: 67 showed greatest sequence homology to a protein sequence having GenBank accession number BAB31307, that was also referred to as murine adult male cecum cDNA, the nucleotide sequence of which is provided in GenBank accession number AK018613. A GAP alignment of BAB31307 with SEQ ID NO: 67 revealed that SEQ ID NO: 67 from amino acids 1 to 578 aligned with BAB31307 from amino acids 1 to 577 with 56% identity and 60% similarity.

The “BLASTP” analysis further revealed that SEQ ID NO: 67 showed sequence homology to many proteins of the CEA family including GenBank accession number AAA51967 which is stated to be human carcinoembryonic antigen mRNA (CEA), SwissProt accession number PSG4_HUMAN stated to be human pregnancy-specific beta-1-glycoprotein 4 precursor, and GenBank accession number AAA51826 stated to be biliary glycoprotein I precursor (Homo sapiens). Since similarity in protein sequence suggests shared function SEQ ID NO: 67 is presumed to share at least some functional similarity with these similar sequences.

A Gap alignment of AAA51967 with SEQ ID NO: 67 revealed that SEQ ID NO: 67 from amino acid 1 to amino acid 461 aligned with AAA5 1967 from amino acids 242 to 701 with 33% identity and 40% similarity. A Gap alignment of PSG4_HUMAN with SEQ ID NO: 67 from amino acids 1 to 438 aligned with PSG4_HUMAN from amino acids 1 to 419 with 33% identity and 40% similarity. A Gap alignment of AAA51826 with SEQ ID NO: 67 revealed that SEQ ID NO: 67 from amino acid 1 to 439 aligns with AAA51826 from amino acids 92 to 526 with 32% identity and 39% similarity.

The “BLASTP” analysis also revealed that SEQ ID NO: 67 showed sequence homology to the following proteins: CAA34405 stated to be TM3-CEA protein [Homo sapiens]; CAA34404 stated to be TM1-CEA [Homo sapiens]; CAA02706 stated to be an unnamed protein product [Homo sapiens]; AAA62835 stated to be carcinoembryonic antigen [Homo sapiens]; AAA51963 stated to be carcinoembryonic antigen precursor [Homo sapiens]; CAA34474 stated to be pCEA80-11 protein (647 AA) [Homo sapiens]; PSG3_Human stated to be pregnancy-specific beta-1-glycoprotein 3 precursor (psbg-3) (carcinoembryonic antigen SG5); AAC60584 stated to be pregnancy-specific beta 1-glycoprotein, PSG {clone hIS25} [human, colon, Peptide, 428 aa] [Homo sapiens]; CAA01646 stated to be trophoblast membrane expressed protein [Homo sapiens]; AAA52606 stated to be pregnancy-specific beta-1 glycoprotein precursor [Homo sapiens]; AAA60960 stated to be carcinoembryonic antigen SG9 [Homo sapiens]; AAA60204 stated to be fetal liver non-specific cross-reactive antigen precursor protein [Homo sapiens]; AAA60203 stated to be PSG11 [Homo sapiens]; CAA35612 stated to be pregnancy-specific beta-1 glycoprotein (AA 1-426) [Homo sapiens]; B35334 stated to be pregnancy-specific beta-1-glycoprotein 7 precursor; AAC18437 stated to be biliary glycoprotein g precursor; C30127 stated to be transmembrane carcinoembryonic antigen 3 precursor—human; and S34338 stated to be biliary glycoprotein F—mouse and P16573 (ECTO_RAT) stated to be ECTO_ATPase precursor (CELL-CAM 105) (C-CAM 1 05) (atp-dependent taurocolate-carrier protein) (GP110).

A search using HMM and the Pfam database as described above revealed several protein motifs in SEQ ID NO: 67. The immunoglobulin domain model PF00047 was found to occur four times within SEQ ID NO: 67 with an overall matching score of 102.18. The first occurrence of the immunoglobulin domain within SEQ ID NO: 67 is from amino acids 83 through 140 similar to the PF00047 model from amino acids 1 through 45. The second match is from SEQ ID NO: 67 amino acids 182 through 232 to the PF00047 model from amino acids 1 through 45. The third match is from SEQ ID NO: 67 amino acids 271 through 326 to the PF00047 model from amino acids 3 through 45. The last is from SEQ ID NO: 67 amino acids 368 through 418 to the PF00047 model from amino acids 1 through 45. Immunoglobulin domains are implicated in protein-protein and protein-ligand interactions. CEA molecules have variable numbers of immunoglobulin domains in their extracellular regions and are members of the immunoglobulin superfamily (review in Hammerstrom, ibid).

An unannotated Pfamb motif was found to match SEQ ID NO: 67 twice with an overall score of 60.96. SEQ ID NO: 67 from amino acids 72 to 157 aligned with amino acids 1 to 87 of the Pfamb motif, and SEQ ID NO: 67 from amino acids 257 to 343 aligned with amino acids 1 through 87 of the Pfamb motif. An investigation of the molecules used to generate this motif as described above revealed that it was generated from pregnancy specific glycoprotein beta-1 and its variants, members of the CEA family. The motif was interspersed with the immunoglobulin domains, comparable to its location within SEQ ID NO: 67.

A second unannotated Pfamb was found to match SEQ ID NO: 67 with a score of 35.10. SEQ ID NO: 65 from amino acids 441 to 476 aligned with amino acids 1 through 36 of the Pfamb motif. An investigation of the molecules used to generate this motif as described above revealed that it was generated from 18 sequences all from CEA family members including: GenBank accession numbers P13688 stated to be human biliary glycoprotein 1 precursor; Q15600 stated to be TM2-CEA precursor; P31809 stated to be murine biliary glycoprotein 1 precursor; Q03715 stated to be nonspecific cross-reacting antigen; P16573 stated to be rat ecto-ATPase precursor; and P40198 stated to be human carcinoembryonic antigen CGM1 precursor. The motif. flanks and spans the transmembrane domain in all of these molecules, comparable to its location within SEQ ID NO: 67.

The PeptideStructure program, used as described above, showed a hydrophobic region in SEQ ID NO: 67 centered around amino acid 457 of sufficient size and hydrophobicity that is likely to function as a transmembrane spanning region. As noted above this region demonstrates a shared motif including the transmembrane region and its flanking sequence with members of the CEA family. The sequences N-terminal to these amino acids containing the immunoglobulin domains, described above, likely constitute an extracellular portion of the molecule. The amino acids C-terminal to this predicted transmembrane domain are likely to form a cytoplasmic domain.

The PeptideStructure program, as described above, also identified a number of potential sites for N-linked glycosylation within the predicted extracellular portion of SEQ ID NO: 67 with strong sites at asparagine residues at amino acids 96, 105, 280, 306, 368, 415 and 513 and a weak site at 317.

The PeptideSort program, as described above, showed that SEQ ID NO: 67 had a molecular weight of 63,581.46 Daltons and an isoelectric point of 5.95.

The cytoplasmic domains of CEA family members human biliary glycoprotein (CEACAMI) and mouse homologs C-CAM1 and C-CAM2 contain binding sites for calmodulin. All three of these molecules share a calmodulin-binding site in the cytoplasmic domain adjacent to the transmembrane domain (Edlund et al., J Biol Chem, 271:1393-1399). SEQ ID NO: 67 shared some sequence conservation in this region from amino acid 468 through 481 ‘FLYIRNARRPSRKT’ (SEQ ID NO: 74) including two charged amino acids at 479 and 480. Both murine homologs contain a second calmodulin-binding site closer to the C-terminus of the cytoplasmic tail that was not found in human biliary glycoprotein. A minimal calmodulin-binding motif ‘Hydrophobic-Q-X3-R’ (Aitken, Molecular Biotechnology, 12:241-53 (1999)) was found in a comparable location in SEQ ID NO: 67 from amino acids 517 to 522 ‘LQGRIR’ (SEQ ID NO: 75). Human biliary glycoprotein forms homodimers and this process is regulated by calmodulin (Edlund et al., J Biol Chem, 271:1393-1399). A similar process may be inferred from sequence similarity and binding motifs found in SEQ ID NO: 67.

The serine found at amino acid 539 of SEQ ID NO: 67 matched the consensus for phosphorylation targets of proline-directed cell-cycle kinases ‘S/T-P-X-K/R′ (Aitken, 1999, ibid) having ‘SPWK’ (SEQ ID NO: 76) from amino acids 539 through 542. SEQ ID NO: 67 has a match to the consensus motif ‘Y-X-X-hydrophobic’ to which SH2 domains can bind when the tyrosine is phosphorylated (Aitken, 1999, ibid) from amino acids 511 through 514 ‘YCNI’ (SEQ ID NO: 77).

A Gap alignment of SEQ ID NO: 66 to SEQ ID NO: 1 revealed that SEQ ID NO: 66 had 551 nucleotides at the 5′ end not found in SEQ ID NO: 1. SEQ ID NO: 66 from nucleotides 552 to 1829 aligned with SEQ ID NO: 1 from nucleotides 2 to 1278 at 100% identity. SEQ ID NO: 66 from nucleotides 1830 to 1992 had little homology to SEQ ID NO: 1.

A Gap alignment of SEQ ID NO: 67 to SEQ ID NO: 2 revealed that SEQ ID NO: 67 was longer than SEQ ID NO: 2 on the 5′ amino terminus having 179 additional amino acids not found in SEQ ID NO: 2. SEQ ID NO: 67 aligned from amino acid 180 to 567 to SEQ ID NO: 2 exactly from 1 to 388. SEQ ID NO: 66 from amino acid 568 to 578 had little homology to SEQ ID NO: 2.

A Gap alignment of SEQ ID NO: 66 to SEQ ID NO: 4 revealed that SEQ ID NO: 66 from 1 to 180 had little homology to SEQ ID NO: 4 from nucleotides 1 to 1663. SEQ ID NO: 66 from 191 to 1436 aligned with SEQ ID NO: 4 from 1664 to 2918 with nearly 100% identity having only two nucleotide differences where SEQ ID NO: 66 at nucleotide 450 has an adenine and SEQ ID NO: 4 has a guanine at corresponding nucleotide 1933 and where SEQ ID NO: 66 at nucleotide 1085 has a cytosine and SEQ ID NO: 4 has a guanine at corresponding nucleotide 2568. SEQ ID NO: 66 has an insertion from nucleotides 1436 to 1462 between corresponding nucleotides 2918 and 2919 of SEQ ID NO: 4. SEQ ID NO: 66 from nucleotides 1463 to 1555 aligned with SEQ ID NO: 4 from 2921 to 3011. SEQ ID NO: 66 from nucleotides 1556 to 1667 had little homology to SEQ ID NO: 4.

A Gap alignment of SEQ ID NO: 67 to SEQ ID NO: 5 revealed that SEQ ID NO: 67 was shorter than SEQ ID NO: 5 on the 5′ amino terminus having 18 amino acids with little homology to amino acids 1 through 555 of SEQ ID NO: 5. SEQ ID NO: 67 aligned from amino acid 19 to 436 to SEQ ID NO: 5 from amino acids 556 to 973 exactly with a single amino acid difference at 108 where SEQ ID NO: 67 had an isoleucine and SEQ ID NO: 5 had a valine at the corresponding amino acid 645. SEQ ID NO: 67 was found to have a small insertion from amino acids 437 through 445 between amino acids 973 and 974 of SEQ ID NO: 5. SEQ ID NO: 67 from amino acids 446 through 476 matched exactly to SEQ ID NO: 5 from amino acids 974 to 1004. SEQ ID NO: 67 from amino acids 477 to 509 had little homology to SEQ ID NO: 5 from amino acids 1005 to 1033.

A Gap alignment of SEQ ID NO: 66 to SEQ ID NO: 54 revealed that SEQ ID NO: 66 from nucleotides 1 to 1702 aligned with SEQ ID NO: 54 from nucleotides 185 to 1885 nearly 100% identity having only a single nucleotide difference at position 1537 where SEQ ID NO: 66 has a adenosine and SEQ ID NO: 54 has a guanine at corresponding position 1721. SEQ ID NO: 66 has no homology to SEQ ID NO: 54 from nucleotides 1886 to 1922. SEQ ID NO: 66 from nucleotides 1703 to 1832 aligned to SEQ ID NO: 54 from 1923 to 2052 with 100% identity. SEQ ID NO: 66 from nucleotides 1833 to 1980 had little homology to SEQ ID NO: 54 from nucleotides 2053 to 2147.

A Gap alignment of SEQ ID NO: 67 to SEQ ID NO: 55 revealed that SEQ ID NO: 67 aligned from amino acid 1 to 525 to SEQ ID NO: 54 from amino acids 1 to 525 with nearly 100% identity having a single amino acid difference at 470 where SEQ ID NO: 67 has a tyrosine and SEQ ID NO: 55 has a cysteine at corresponding position 470. SEQ ID NO: 67 had no homology from amino acids 525 to 526 between amino acids 525 and 537 of SEQ ID NO: 55. SEQ ID NO: 67 from amino acids 526 to 567 aligned with SEQ ID NO: 55 from amino acids 537 to 579 with 100% identity. SEQ ID NO: 67 had little homology from 568 to 578 with little identity to SEQ ID NO: 55 from 580 to 585.

A Gap alignment of SEQ ID NO: 66 to SEQ ID NO: 64 revealed that SEQ ID NO: 66 from nucleotides 57 to 1829 aligned with SEQ ID NO: 64 from nucleotides 2 to 1774 with 100% identity. SEQ ID NO: 66 from nucleotides 1830 to 1992 had little homology to SEQ ID NO: 64 from nucleotides 1775 to 1931.

A Gap alignment of SEQ ID NO: 67 to SEQ ID NO: 65 revealed that SEQ ID NO: 67 aligned from amino acid 1 to 567 to SEQ ID NO: 64 from amino acids 1 to 567 exactly. SEQ ID NO: 67 had little homology from 568 to 578 with little identity to SEQ ID NO: 64 from 580 to 584.

SEQ ID NO: 67 has half of an N-terminal V-type immunoglobulin domain, and then four C-type immunoglobulin domains, of alternating A and B subtypes. An N-terminal Ig domain followed by alternating A and B subtypes Ig domains is characteristic of the CEA family. A comparison of the domain structure of SEQ ID NO: 67 with a known CEA family member CEACAM1 is given in FIG. 3.

Based upon the similarity of protein sequence to other CEA molecules, shared HMM motifs, similar patterns of Ig domain distribution, localization to chromosome 19 and calmodulin binding motifs, SEQ ID NO: 66 encodes a novel member of the CEA family. SEQ ID NO: 67 is a novel member of the CEA family. SEQ ID NO: 66 and its expressed polypeptide SEQ ID NO: 67 are useful as tumor markers. However, even absent differential expression in tumors, a polypeptide or polynucleotide is useful as a tumor marker when it shows tissue specificity. Some CEA family members have been proven useful for immunolocalization of tumor tissue (, Nakopoulou et al., Dis Colon Rectum, 26:269-74 (1983)), in particular for radioimmunosurgery (Bertoglio et al., Seminars in Surgical Oncology), and for immunotherapy (Khare et al., Cancer Research, 61:370-5 (2001); Buchegger et al., Int J Cancer, 41:127-134 (1988)).

While SEQ ID NOs: 66 and 67 share sequence similarities to other CEA family members, no cross-reactivity to known family members is expected under high stringency nucleic acid hybridization due to the extent of unique sequence. Specific antibodies that do not cross-react with known family members can be raised based upon the pattern of antigenic sites present in the polypeptides encoded by the polynucleotide in cDNA SEQ ID NO: 66. Since SEQ ID NO: 66 was isolated from human prostate tissue, shows strong expression in that tissue, and was isolated as a variant of SEQ ID NO: 1, 66 and the polypeptide it encodes SEQ ID NO: 67 are useful as biomarkers of prostate tissue and as markers for metastasized prostate tissue.

### Example 21

### Exon Structure of Clone PCEA3

The exon structure of SEQ ID NO: 64 is diagramed in FIG. 2F, and shown in Table 2 (FIG. 8). The cDNA sequence is SEQ ID NO: 66 and comprises of 11 exons: SEQ ID NOs: 56, 26, 58, 30, 52, 34, 42, 44, 46, 48 and 68.

SEQ ID NO: 68, a unique exon present in SEQ ID NO: 64, has utility as biomarker for cancer, since it can be used as a probe to detect the levels of SEQ ID NO: 64 expressed in biopsied tissues or postoperatively in excised tumors. The peptides encoded by exons are SEQ ID NOs: 57, 27, 59, 31, 33, 35, 43, 45, 47, 49 and 69, respectively. Antigenicity analysis was performed using PlotStructure as described above. The peptides encoded by each exon contained regions of positive antigenicity demonstrating that they were each good substrates for the generation of antibodies. Antibodies to each of these peptides can be used to detect SEQ ID NO: 67 in tissue in vivo or in vitro.

### Example 22

### Expression of PCEAs in Tumor Tissues

The presence of PCEAs in normal and malignant prostate tissue was demonstrated by Northern analysis. Tissue biopsies from normal and malignant prostate were obtained from Lahey Clinic. Tissue was homogenized in TRIZOL (Cat. No: 15596-018 GIBCO-BRL, Bethesda, Md.) reagent at a concentration of 2 g tissue/20 ml reagent with a Polytron probe (Brinkmann Instruments, Westbury, N.Y.). The homogenate was incubated briefly at room temperature. Four mL of chloroform were added and again incubated briefly at room temperature prior to centrifugation. The aqueous phase was transferred to a new tube and precipitated with isopropyl alcohol. The RNA was then resuspended in 0.5% SDS. Northern blots were prepared using 10 g of total RNA/lane.

Probe was made by random priming using a High Prime DNA labeling kit (Cat. No: 1585584, Roche Diagnostics, Indianapolis, Ind.) according to manufacturer's instructions using the full DNA sequence given in SEQ ID NO: 1. Hybridization was overnight at 45° C. according to manufacturer's instructions in Ambion Ultrahyb (Cat. No: 8670). The blot was washed at 50° C. for 1 hour in 0.1×SSC (in “Molecular Cloning: A Laboratory Manual,” Sambrook J, Fritsch E F and Maniatis T, Cold Spring Harbor Laboratory Press (1989)) and 0.1% sodium dodecyl sulfate.

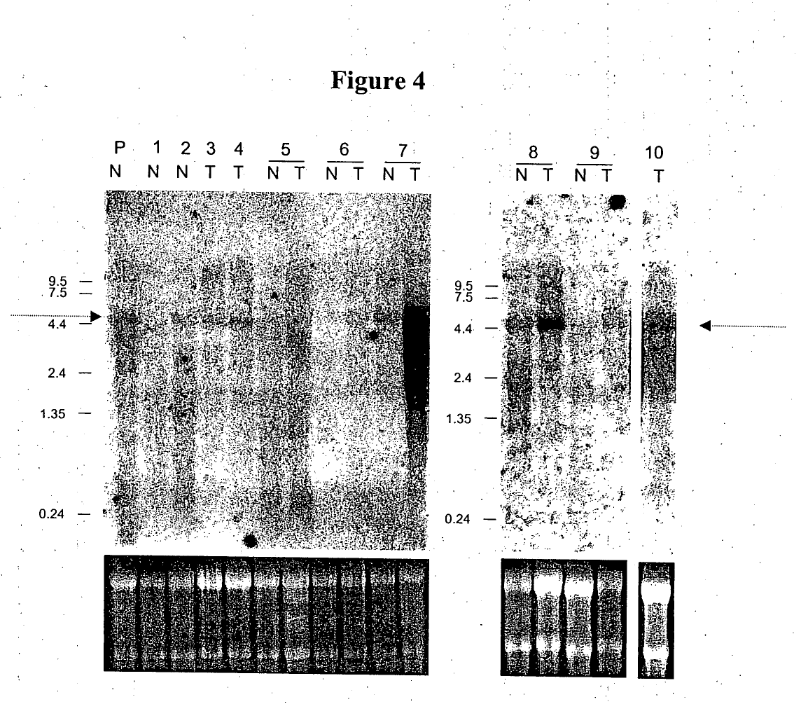

The results are shown in FIG. 4. The results demonstrated the presence of polynucleotide sequences of the present invention in normal prostate and prostate tumor samples. Differential expression in tumor tissue is not required for utility as an imaging agent or as a biomarker for normal and tumor prostate tissue. Utility as a cytotoxic agent target also does not require differential expression in tumor versus normal tissue, since the existing therapies for prostate cancer include the destruction of the normal, as well as tumor, from prostate tissue.

Semi-quantitative RT-PCR was also used to demonstrate the presence of expressed sequences of the present invention in normal prostate and prostate tumor tissues. Reverse transcription of 2 mg of total RNA from eight samples was carried out for 1 hour at 42° C. in 50 ml of RT buffer (No: Y00146, Invitrogen, Carlsbad, Calif.), supplemented with 0.5 mM of each dNTP (No: 1969064, Roche, Indianapolis, Ind.), 10 mM dithiothreitol, 2 Units of ribonuclease inhibitor (Superase Inhibitor No: 2694, Ambion, Austin, Tex.) 500 units of SuperScript II reverse transcriptase (No: 18064-022, Invitrogen, Carlsbad, Calif.) and 500 ng of a random hexamer for priming. The reaction was stopped by heat denaturation at 70° C. for 15 min., followed by a 20 mins. incubation at 37° C. with RNAse H.

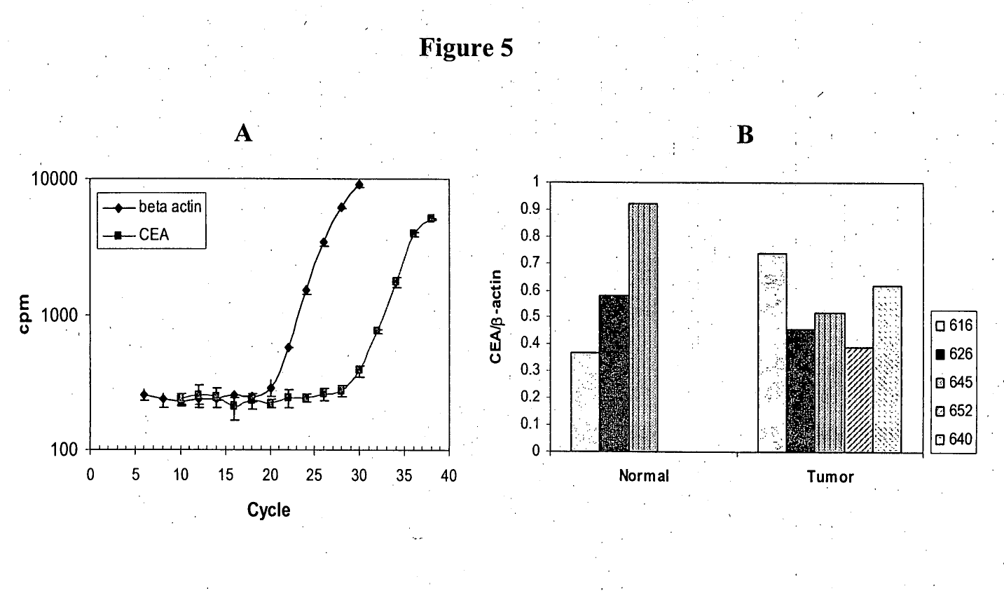

Polymerase chain reaction (PCR) was carried out in a volume of 50 ml, using PCR buffer C (60 mM Tris-HCl, 15 mM (NH4)2SO4, 2.5 mM MgC12, pH 8.5) (PCR Optimizer Kit No: 45-0323, Invitrogen, Carlsbad, Calif.), 1 Unit of Taq polymerase (No: 201203, QIAGEN, Valencia, Calif.), 0.24 mM each dNTP (No: 1969064, Roche, Indianapolis, Ind.), 0.15 ml [32 P]- -dATP (6000 Ci/mmol, NEN), 0.5 mM of each primer, and 1 mL of template (from a 50 ml RT reaction). The primers used for pCEA were 5′-CATCGCTGGTATTGTCATCGG-3′ (SEQ ID NO: 82) and 5′-CGTCTGGCATTTCTGATGTAGAG-3′ (SEQ ID NO: 83). The primers used for beta-actin were 5′-GGACTTCGAGCAAGAGATGG-3′ (SEQ ID NO: 84) and 5′-TGAAGGTAGTTTCGTGGATGC-3′ (SEQ ID NO: 85). Thermal cycling was performed in a MJ-Research thermal cycler (model PTC-200, Watertown, Mass.) as follows: (1) initial denaturation at 94° C. for 2 minutes, (2) cycling for the indicated number of cycles (see below) between 94° C. for 30 seconds, the annealing temperature (see below) for 30 seconds and 72° C. for 40 seconds, (3) final extension at 72° C. for 5 minutes. The number of cycles was chosen so that amplification remained well within the linear range, as assessed by TCA-precipitable counts from triplicate samples, obtained every 2 cycles from cycles 6-38 (see FIG. 5A). For PCEA, the number of cycles was 33; for beta-actin the number of cycles was 25. The specificity of the PCR reactions was verified for these primers by restriction mapping and sequencing of the PCR products. In all cases, PCR amplification was shown to be dependent on reverse transcription of RNA templates. Each tissue sample was analyzed in triplicates. At the end of the reaction, PCR products in a 4 ml sample were quantified by Cerenkov counts in a Beckman (Irvine, Calif.) LSI0001 scintillation counter. The levels of pCEA were normalized to bactin and expressed as the ratio pCEA/bactin.

The portion of the PCEA molecules amplified corresponds to the transmembrane domain and surrounding region (see FIG. 3 for illustration of the domains) which is common to SEQ ID NOs: 1, 64, 54 and 66. The amplified fragment matches SEQ ID NO: 64 from nucleotide 1414 to nucleotide 1500, and to the corresponding sequences in SEQ ID NOs: 1, 54 and 66. The presence of PCEA was detected by semi-quantitative RT PCR in all normal and tumor samples analyzed. The results are diagramed in FIG. 5B.

Expression of polynucleotide sequences of the present invention was further demonstrated by PCR in a cell line derived from the bone metastasis of a primary prostate tumor. The primers used were: 5′-CTG CCA TAG AGC AGA AGG ACA TGG-3′ (SEQ ID NO: 86) and 5′-GGA TGA TTA GGG TCC TGT TGT CAG G-3′ (SEQ ID NO: 87). The cell lines used were:

a) DU-145: Isolated from brain metastasis, after carcinoma of the prostate.

b) LN CaP: Lymph node metastasis from prostate carcinoma.

c) PC-3: Bone metastasis, from grade 4 prostate adenocarcinoma.

d) CRL-2220; Prostate adenocarcinoma with Gleason score 4/4.

e) CRL-2422; Bone metastasis, from an African-American male with androgen-independent prostate adenocarcinoma.

f) RT-112; grade II bladder tumor.

g) RT-4; grade I bladder tumor.

h) J-82; poorly-differentiated late-stage bladder cancer.

i) Um-Uc-3; transitional cell carcinoma of the urinary bladder.

PCR was conducted using the kit according to manufacturers instructions with cDNA from each of the cell lines. The following cycles were used:

| | |
|---|---|
| Step 1 | 95° C. for 1 min. |
| Step 2 | 95° C. for 45 sec. |
| Step 3 | 65° C. for 30 sec. |
| Step 4 | 72° C. for 50 sec. |
| Step 5 | REPEAT steps 2-4 for 34 times. |
| Step 6 | 72° C. for 5 min. |
| Step 7 | 4° C. indefinitely. |

The 20 ml of PCR product was run on a 1.0% agarose gel along with a 100-bp DNA ladder, as a marker. The PCR product produced encodes part of the A1 Ig domain, all of the B1 Ig domain and part of the A2 Ig domain, which SEQ ID NOs: 64, 54 and 66 share in common (see FIG. 3 for illustration of the domains). The amplified fragment matches SEQ ID NO: 64 from nucleotide 306 through nucleotide 1007 and to the corresponding sequence in SEQ ID NOs: 54 and 66.

Three repeat experiments, using above mentioned cell lines, indicated the presence of a 700 bp transcript only in CRL 2422 (ATCC), a prostate cancer cell line, derived from a bone metastasis of a 63 year old African-American male with androgen-independent adenocarcinoma of the prostate. Bone is the most common site of metastasis for prostate cancer. Expression was absent in all of the bladder cancer control cell lines.

The expression of PCEAs in prostate, prostate tumor and bone metastasis derived from prostate cancer demonstrated the utility of the sequences of the present invention as markers for prostate tissue, prostate tumor tissue and metastases from prostate tumor. These markers can be used for radioimmunoguided surgery, and as an imaging agent for the diagnosis and prognosis of prostate cancer. These results also demonstrated the utility of the sequences of the present invention as targets for antibody mediated therapies, such as the direction of a cytotoxic agent to prostate and prostate tumor tissue, since expression of the molecules were maintained in these tissues.

### Example 23

### Protein Expression

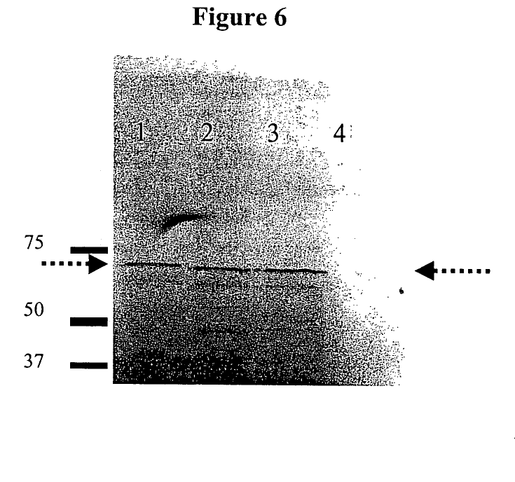

Expression of the polypeptides SEQ ID NOs: 55, 65 and 67 was demonstrated using a TNT Couple Reticulocyte Lysate System (Catalog No: L4611, Promega, Madison, Wis.). SEQ ID NO: 54 was in pSportI, an expression vector, and SEQ ID NOs: 64 and 66 were subcloned into pSportI vector using standard methods (“Molecular Cloning: A Laboratory Manual,” Sambrook J, Fritsch E F, and Maniatis T, Cold Spring Harbor Laboratory Press (1989)). The TNT in vitro translation kit was used according to manufacturer's instructions to express the encoded polypeptides. The expressed polypeptides were run on a protein gel using standard methods. An autoradiogram of the results is shown in FIG. 6. A full-length polypeptide was produced for each construct. Lane 1 revealed SEQ ID NO: 65 was a protein of approximately 64 kDa. Lane 2 revealed that SEQ ID NO: 55 was a protein of approximately 64 kDa. Lane 3 revealed that SEQ ID NO: 67 was a protein of approximately 64 kDa. The control in lane 4 revealed that no proteins produced without the SEQ ID NOs: 54, 64 or 66 templates. The observed molecular weights are in good agreement with the calculations provided by PeptideSort, given above.

### Example 24

### Demonstration of Protein-Protein Interaction

Protein-protein interactions were assayed following the guidelines provided in Fields and Song, Nature, 340:245-246 (1989). The principle of the assay is based on the ability to join two parts of a transcriptional activator to get transcription of a marker gene. Nucleic acid encoding fragments of a protein of interest (the baits) are fused to a portion of a transcriptional activator and screening for interactions with another fusion protein. The other fusion protein consists of another portion of the activator fused to potentially interacting molecules (the prey). When the bait and prey bind directly to each other, transcription is activated and can be detected by use of a reporter gene in yeast.

A yeast two-hybrid assay was performed using pooled fragments of PCEA protein expressed as baits. cDNAs from human prostate (Clontech, Catalog No: HL4037AH) was used as the prey and was screened against the pooled bait according to manufacturer's instructions. Among the prey molecules obtained that bound directly to the fragments of expressed PCEA proteins was p7-2b5 which encoded a protein fragment of PCEA. The cDNA encoding p7-2-5 was in common to SEQ ID NOs: 54, 64 and 66 and matched SEQ ID NOs: 64 from nucleotide 72 through nucleotide 473. P7-2b5 encoded polypeptide that included the N-terminal half V-type Ig domain and the A1 Ig domain (see FIG. 3 for illustration of the domains). These domains were in common to SEQ ID NOs: 55, 65 and 67. This demonstrated the ability of these polypeptides to interact.

### Example 25

### Cytoplasmic Domain Variant Obtained by PCR

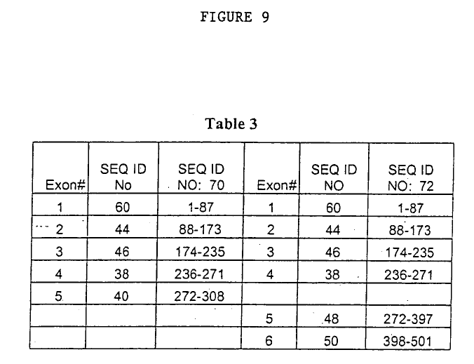

Two additional splicing variants for the cytoplasmic region of PCEA were demonstrated by PCR. The first of these is SEQ ID NO: 70 and its encoded polypeptide is SEQ ID NO: 71. SEQ ID NO: 70 comprised exons SEQ ID NOs: 60, 44, 46, 38 and 40, shown in FIG. 2G and in Table 3 (FIG. 9).

The cytoplasmic domains of CEA family members human biliary glycoprotein (CEACAM1) and mouse homologs C-CAM1 and C-CAM2 contain binding sites for calmodulin. All three of these molecules share a calmodulin-binding site in the cytoplasmic domain adjacent to the transmembrane domain (Edlund et al., J Biol Chem, 271:1393-1399). SEQ ID NO: 71 shared some sequence conservation in this region from amino acid 21 through 34° FLCIRNARRPSRKT′ (SEQ ID NO: 80) including two charged amino acids at 32 and 33. Both murine homologs contain a second calmodulin-binding site closer to the C-terminus of the cytoplasmic tail that was not found in human biliary glycoprotein. A minimal calmodulin-binding motif ‘Hydrophobic-Q-X3-R’ (Aitken, Molecular Biotechnology, 12:241-53 (1999)) was found in a comparable location in SEQ ID NO: 73 from amino acids 70 to 75 ‘LQGRIR’ (SEQ ID NO: 75). Human biliary glycoprotein forms homodimers and this process is regulated by calmodulin (Edlund et al., J Biol Chem, 271:1393-1399). A similar process may be inferred from sequence similarity and binding motifs found in SEQ ID NO: 71.

No consensus for phosphorylation targets of proline-directed cell-cycle kinases ‘S/T-P-X/K/R’ (Aitken, 1999, ibid) was found in SEQ ID NO: 71. SEQ ID NO: 71 had two matches to the consensus motif ‘Y-X-X-hydrophobic’ to which SH2 domains can bind when the tyrosine is phosphorylated (Aitken, 1999, ibid). These motifs were found from amino acids 64 through 67 ‘YCNI’ (SEQ ID NO: 77) and from amino acids 89 through 92 ‘YEGL’ (SEQ ID NO: 88).

### Example 26

### Cytoplasmic Domain Splicing Variant Obtained by PCR

The second cytoplasmic domain variant obtained by PCR is SEQ ID NO: 72. It comprises exons SEQ ID NOs: 60, 44, 46, 38, 48 and 50. The exon structure of SEQ ID NO: 72 is diagramed in FIG. 2H, and given in Table 3 (FIG. 9).

The cytoplasmic domains of CEA family members human biliary glycoprotein (CEACAMI) and mouse homologs C-CAM1 and C-CAM2 contain binding sites for calmodulin. All three of these molecules share a calmodulin-binding site in the cytoplasmic domain adjacent to the transmembrane domain (Edlund et al., J Biol Chem, 271:1393-1399). SEQ ID NO: 73 shared some sequence conservation in this region from amino acid 21 through 34° FLCIRNARRPSRKT’ (SEQ ID NO: 80) including two charged amino acids at 32 and 33. Both murine homologs contain a second calmodulin-binding site closer to the C-terminus of the cytoplasmic tail that was not found in human biliary glycoprotein. A minimal calmodulin-binding motif ‘Hydrophobic-Q-X3-R’ (Aitken, Molecular Biotechnology, 12:241-53 (1999)) was found in a comparable location in SEQ ID NO: 73 from amino acids 70 to 75 ‘LQGRIR’ (SEQ ID NO: 75). Human biliary glycoprotein forms homodimers and this process is regulated by calmodulin (Edlund et al., J Biol Chem, 271:1393-1399). A similar process may be inferred from sequence similarity and binding motifs found in SEQ ID NO: 73.

The serine found at amino acid 104 of SEQ ID NO: 73 matched the consensus for phosphorylation targets of proline-directed cell-cycle kinases ‘S/T-P-X-K/R’ (Aitken, 1999, ibid ) having ‘SPWK’ (SEQ ID NO: 76) from amino acids 104 through 107. SEQ ID NO: 73 also had two matches to the consensus motif ‘Y-X-X-hydrophobic’ to which SH2 domains can bind when the tyrosine is phosphorylated (Aitken, 1999, ibid). These motifs were found from amino acids 64 through 67 ‘YCNI’ (SEQ ID NO: 77) and from amino acids 131 through 134 ‘YEEL’ (SEQ ID NO: 78).

### Example 27

### Confirmation of Predicted Exons by PCR

There are predicted exons that have been confirmed by PCR. They are: SEQ ID NOs: 14, 16,18, 20, 22 and 40. SEQ ID NO: 22 has three possible exon starts.

### DESCRIPTION OF THE SEQUENCE LISTING

SEQ ID NO: 1 is the polynucleotide sequence from clone 128375. SEQ ID NO: 2 is an amino acid sequence encoded by SEQ ID NO: 1. SEQ ID NO: 3 is an alternative amino acid sequence encoded by SEQ ID NO: 1. These are discussed further in Example 9.

SEQ ID NO: 4 is a predicted polynucleotide sequence. SEQ ID NO: 5 is the deduced amino acid sequence encoded by SEQ ID NO: 4. These sequences are discussed further in Example 10.

SEQ ID NO: 54 is a polynucleotide sequence from clone (427896) PCEA2. SEQ ID NO: 55 is the deduced amino acid sequence encoded by SEQ ID NO: 54. These are discussed further in Example 17.

SEQ ID NO: 64 is a polynucleotide sequence from clone (457507) PCEA1-FL. SEQ ID NO: 65 is the deduced amino acid sequence encoded by SEQ ID NO: 64. These are discussed in Example 19.

SEQ ID NO: 66 is a polynucleotide sequence from clone (451608) PCEA3. SEQ ID NO: 67 is the deduced amino acid sequence encoded by SEQ ID NO: 66. These are discussed in Example 21.

SEQ ID NO: 70 is a polynucleotide sequence from a PCR product 387. SEQ ID NO: 71 is the deduced amino acid sequence encoded by SEQ ID NO: 70. These are discussed in Example 26.

SEQ ID NO: 72 is a polynucleotide sequence from a PCR product 503. SEQ ID NO: 73 is the deduced amino acid sequence encoded by SEQ ID NO: 72. These are discussed in Example 27.

SEQ ID NOs: 30, 34, 42, 44, 46, 48, 50, 52 and partial 28 are the exons comprising SEQ ID NO: 1. SEQ ID NOs: 31, 35, 43, 45, 47, 49, 51, 53 and partial 29 are the amino acid sequences encoded by the respective exons.

SEQ ID NOs: 6, 8, 10, 12, 14, 16, 18, 20, 22, 24, 26, 28, 30, 32, 34, 36, 38, and 40are the exons comprising SEQ ID NOs: 4. SEQ ID NOs: 7, 9, 11, 13, 15, 17, 19, 21, 23, 25, 27, 29, 31, 33, 35, 37, 39 and 41 are the amino acid sequences encoded by the respective exons.

SEQ ID NOs: 26, 30, 34, 38, 44, 46, 48, 52, 56, 58, 60 and 62 are the exons comprising SEQ ID NO: 54. SEQ ID NOs: 27, 31, 35, 53, 45, 47, 49, 33, 57, 59, 61 and 63 are the amino acid sequences encoded by the respective exons.

SEQ ID NOs: 26, 30, 34, 42, 44, 46, 48, 50, 52, 56 and 58 are the exons comprising SEQ ID NO: 64. SEQ ID NOs: 27, 31, 35, 43, 45, 47, 49, 51, 53, 57 and 59 are the amino acid sequences encoded by the respective exons.

SEQ ID NOs: 26, 30, 34, 42, 44, 46, 48, 52, 56, 58 and 68 are the exons comprising SEQID NO: 66. SEQ ID NOs: 27, 31, 35, 43, 45, 47, 49, 53, 57, 59 and 69 are the amino acid sequences encoded by the respective exons.

SEQ ID NOs: 60, 44, 46, 38 and 40 are the exons comprising SEQ ID NO: 70. SEQ ID NOs: 61, 45, 47, 39 and 41 are the amino acid sequences encoded by the respective exons.

SEQ ID NOs: 60, 44, 46, 38, 48 and 50 are the exons comprising SEQ ID NO: 72. SEQ ID NOs: 61, 45, 47, 39, 49 and 51 are the amino acid sequences encoded by the respective exons.

SEQ ID NOs: 74 and 80 are calmodulin binding sites present in polypeptide sequences of the present invention.

SEQ ID NO: 75 is a minimal calmodulin binding domain in a polypeptide sequence of the present invention.

SEQ ID NO: 76 is a phorphorylation target in polypeptide sequences of the present invention.

SEQ ID NOs: 77-81 and 88 are SH2 domains in polypeptide sequences of the present invention.

SEQ ID NOs: 82-87 are PCR primers.

While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated polynucleotide selected from the group consisting of;
a) a polynucleotide selected from the group consisting of SEQ ID NOs: 1, 54, 64, 66, 70, and 72;
b) a polynucleotide complementary to any one of the polynucleotides of a);
c) a polynucleotide encoding a polypeptide sequence selected from the group consisting of: SEQ ID NOs: 2, 3, 5, 55, 65, 67, 71, and 73; and
d) a polynucleotide that is 90% identical to any one of the polynucleotides of a), b), and c) using DNA alignment program BLASTN on default parameters, wherein the polynucleotide encodes a CEA protein.

2. An isolated polynucleotide from the group consisting of;
a) a polynucleotide selected from the group consisting of SEQ ID NOs: 6, 8, 10, 12, 14, 16, 18, 20, 22, 24, 26, 28, 30, 32, 34, 36, 38, 40, 42, 44, 46, 48, 50, and 52;
b) a polynucleotide encoding a polypeptide sequence selected from the group consisting of: SEQ ID NOs: 7, 9, 11, 13, 15, 17, 19, 21, 23, 25, 27, 29, 31, 33, 35, 37, 39, 41, 43, 45, 47, 49, 51, and53;and
c) a polynucleotide complementary to any one of the polynucleotides of a) and b).

3. A vector comprising the polynucleotide sequence of claims 1 or 2.

4. A cell transformed with the nucleic acid sequence of claims 1 or 2.

5. A method for producing a CEA polypeptide, comprising;
a) culturing a host cell transformed with the isolated polynucleotide of claims 1 or 2 in a suitable culture medium; and
b) isolating said protein from the culture.

6. A protein produced by the process of claim 5.

7. A kit for use in detecting CEA expression in a biological sample, comprising at least one oligonucleotide probe which selectively binds under high stringency conditions to an isolated nucleic acid comprising a sequence selected from the group consisting of: SEQ ID NOs: 1, 54, 64, 66, 70, and 72, wherein said probe is detectably labeled.

8. The kit of claim 7, wherein the probe is selected from the group consisting of: SEQ ID NOs: 6, 8, 10, 12, 14, 16, 18, 20, 22, 24, 26, 28, 30, 32, 34, 36, 38, 40, 42, 44, 46, 48, 50, and 52.

9. The kit of claim 8, further comprising a positive control, selected from the group consisting of: SEQ ID NOs: 1, 54, 64, 66, 70, and 72.

10. The kit of claim 8, wherein the biological sample comprises prostate cells.

11. The kit of claim 10, wherein the prostate cells are cancer cells.

12. A method for detecting CEA expression in a biological sample, wherein the biological sample comprises RNA, the method comprising;
a) contacting a biological sample with a nucleic acid probe, under conditions such that the nucleic acid probe hybridizes to complementary RNA sequence, if present, in the biological sample, wherein the probe is designed to specifically hybridize any one of SEQ ID NOs: 1, 54, 64, 66, 70, and 72; and
b) detecting specifically hybridized probe, thereby detecting CEA expression in the biological sample.

13. The method of claim 12, wherein the biological sample comprises cells.

14. The method of claim 13, wherein the cells are prostate cells.

15. The method of claim 12, wherein the sample comprises isolated nucleic acids.

16. The method of claim 15, wherein the nucleic acids are immobilized on a solid support.

17. A CEA polypeptide comprising an amino acid sequence selected from the group consisting of:
a) SEQ ID NOs: 2, 3, 5, 55, 65, 67, 71, 73;
b) polypeptides having 80% identity with any one of SEQ ID NOs: 2, 3, 5, 55, 65, 67, 71, 73 using protein alignment program BLASTP under default conditions; and
c) SEQ ID NOs: 7, 9, 11, 13, 15, 17, 19, 21, 23, 25, 27, 29, 31, 33, 35, 37, 39, 41, 43, 45, 47, 49, 51, and 53.

18. An antibody immunospecific for the CEA polypeptide of claim 12.

19. A method for detecting CEA polypeptide in a biological sample, wherein the biological sample comprises polypeptides, the method comprising;
a) contacting a biological sample with a CEA specific antibody, under conditions such that the antibody binds to the CEA protein, if present, in the biological sample, wherein the antibody is specific for any one of SEQ ID NOs: 2, 3, 5, 55, 65, 67, 71, and 73; and
b) detecting specifically bound antibody, thereby detecting CEA protein in the biological sample.

20. The method of claim 19, wherein the biological sample comprises cells.

21. The method of claim 20, wherein the cells are prostate cells.

22. The method of claim 19, wherein the sample comprises isolated proteins.

23. The method of claim 22 wherein the proteins are immobilized on a solid support.
